(19) United States
(12) Patent Application Publication
(10) Pub. No.: US 2026/0159516 A1
(43) Pub. Date: Jun. 11, 2026

(54) COMPOUNDS AS CBP/P300 DEGRADERS AND USES THEREOF

(71) Applicant: Oncopia Therapeutics, Inc. d/b/a/ SK Life Science Labs, King of Prussia, PA (US)

(72) Inventors: Xuqing ZHANG, King of Prussia, PA (US); Jeremy ROACH, King of Prussia, PA (US)

(21) Appl. No.: 19/410,442

(22) Filed: Dec. 5, 2025

Related U.S. Application Data
(60) Provisional application No. 63/728,334, filed on Dec. 5, 2024.

(51) Int. Cl. C07D 471/04; A61K 31/4709; A61K 31/4725

(52) U.S. Cl. CPC C07D 471/04; A61K 31/4709; A61K 31/4725

(57) ABSTRACT

Described herein are compounds of Formula A and pharmaceutically acceptable salts, solvates, and stereoisomers thereof, as well as their uses (e.g., for degrading certain proteins such as p300/CBP proteins).

### RELATED APPLICATION

This application claims the benefit of and priority to U.S. Provisional Application No. 63/728,334, filed Dec. 5, 2024, the contents of which are incorporated herein by reference in their entireties.

### BACKGROUND

Heterobifunctional degraders simultaneously bind a target protein and an E3 ligase complex, resulting in the transfer of ubiquitin and initiating a process ultimately causing the proteasomal degradation of the target protein. By catalyzing the formation of a ternary complex involving an E3 ligase receptor, a protein of interest, and a small molecule, heterobifunctional degraders may yield enhanced substrate specificity. There is an ongoing need for selective heterobifunctional degraders for treating and/or preventing cancer and other diseases responsive to the inhibition or degradation of CBP/p300 proteins. P300-selective degraders can display strong selectivity for p300 over CBP and demonstrate a time-dependent loss of p300, enhancer acetylation, and transcriptional output in cancer cells both in vitro and in vivo with limited toxicity to untransformed cells. Hence, enhanced dependency on p300 is found across numerous cancer lineages and confirm that the mechanism of degrader function is highly dependent on expression of CRBN, providing a foundation that will enable the study of p300-selective degraders in multiple distinct cellular contexts.

This disclosure arises from a need to provide, e.g., further compounds for the degradation of CBP/p300 proteins with improved therapeutic potential (e.g., for treating cancer).

### SUMMARY

In certain aspects, the present disclosure provides compounds of Formula A:

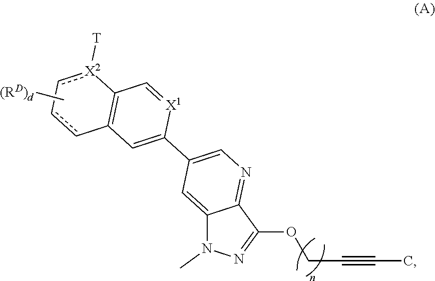

and pharmaceutically acceptable salts, solvates, and stereoisomers thereof, wherein variables T, X¹, X², R^{D}, d, n, and C are described herein.

In certain aspects, the present disclosure provides conjugates of antibodies and compounds of Formula A.

In certain aspects, the present disclosure provides pharmaceutical compositions comprising a compound or conjugate disclosed herein, and one or more pharmaceutically acceptable excipient.

In certain aspects, the present disclosure provides methods of degrading a protein in a subject or biological sample comprising administering a compound or conjugate disclosed herein to the subject or contacting the biological sample with the compound or conjugate disclosed herein.

In certain aspects, the present disclosure provides uses of a compound or conjugate disclosed herein in the manufacture of a medicament for degrading a protein in a subject or biological sample.

In certain aspects, the present disclosure provides compounds and conjugates disclosed herein for use in degrading a protein in a subject or biological sample.

### DETAILED DESCRIPTION

The present disclosure relates to compounds that show activity in degrading certain proteins (e.g., p300/CBP), and pharmaceutical compositions comprising such compounds. Compounds disclosed herein may also be conjugated to antibodies, wherein such conjugates may exhibit, e.g., enhanced delivery properties.

### Compounds of the Present Disclosure

In certain aspects, the present disclosure provides compounds of Formula A:

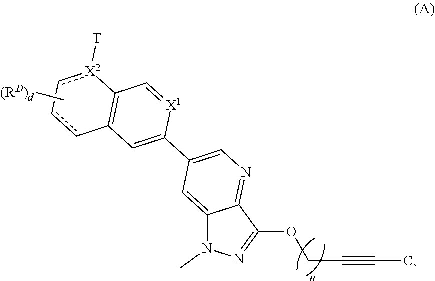

and pharmaceutically acceptable salts, solvates, and stereoisomers thereof, wherein:
each [symbol] independently is a single bond or a double bond;
X¹ is N or CR^{X1};
R^{X1} is hydrogen or C₁-C₆ alkyl, wherein the C₁-C₆ alkyl is optionally substituted with one or more halogen, —CN, —OH, or —NH₂;
X² is N or C, as valency permits;
T is T-1 or T-2:

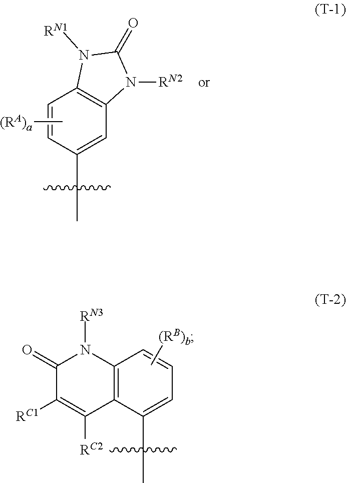

R^{N1} is hydrogen or C₁-C₆ alkyl, wherein the C₁-C₆ alkyl is optionally substituted with one or more halogen, —CN, —OH, or —NH₂;
R^{N2} is hydrogen or C₁-C₆ alkyl, wherein the C₁-C₆ alkyl is optionally substituted with one or more halogen, —CN, —OH, or —NH₂;
R^{N3} is hydrogen or C₁-C₆ alkyl, wherein the C₁-C₆ alkyl is optionally substituted with one or more halogen, —CN, —OH, or —NH₂;
each R^{A} independently is halogen, —CN, —OH, —NH₂, or C₁-C₆ alkyl, wherein the C₁-C₆ alkyl is optionally substituted with one or more halogen, —CN, —OH, or —NH₂;
a is an integer selected from 0 to 3;
each R^{B} independently is halogen, —CN, —OH, —NH₂, or C₁-C₆ alkyl, wherein the C₁-C₆ alkyl is optionally substituted with one or more halogen, —CN, —OH, or —NH₂;
b is an integer selected from 0 to 3;
R^{C1} is hydrogen or C₁-C₆ alkyl, wherein the C₁-C₆ alkyl is optionally substituted with one or more halogen, —CN, —OH, or —NH₂;
R^{C2} is hydrogen or C₁-C₆ alkyl, wherein the C₁-C₆ alkyl is optionally substituted with one or more halogen, —CN, —OH, or —NH₂;
each R^{D} independently is halogen, —CN, —OH, —NH₂, or C₁-C₆ alkyl, wherein the C₁-C₆ alkyl is optionally substituted with one or more halogen, —CN, —OH, or —NH₂;
d is an integer selected from 0 to 5, as valency permits;
n is an integer selected from 1 to 5; and
C is a cereblon (CRBN) type E3 ligase ligand.

In certain aspects, the present disclosure provides compounds of Formula I:

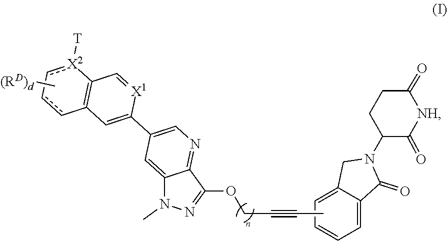

and pharmaceutically acceptable salts, solvates, and stereoisomers thereof.

In certain aspects, the present disclosure provides compounds of Formula I-a:

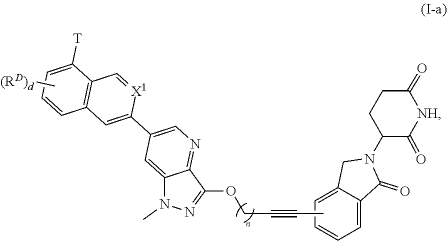

and pharmaceutically acceptable salts, solvates, and stereoisomers thereof.

In certain aspects, the present disclosure provides compounds of Formula I-b:

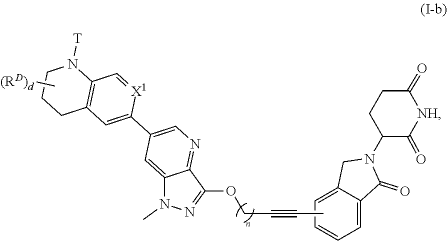

and pharmaceutically acceptable salts, solvates, and stereoisomers thereof.

In certain embodiments, the compound is of Formula I-1:

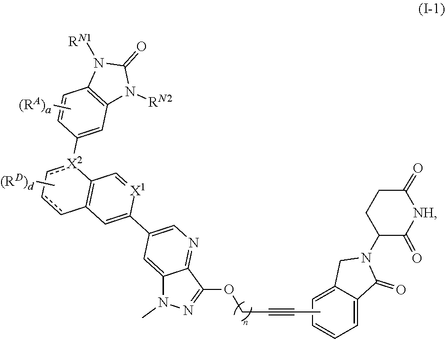

or a pharmaceutically acceptable salt, solvate, or stereoisomer thereof.

In certain embodiments, the compound is of Formula I-1-a:

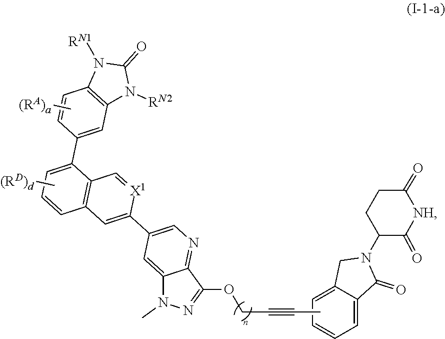

or a pharmaceutically acceptable salt, solvate, or stereoisomer thereof.

In certain embodiments, the compound is of Formula I-2:

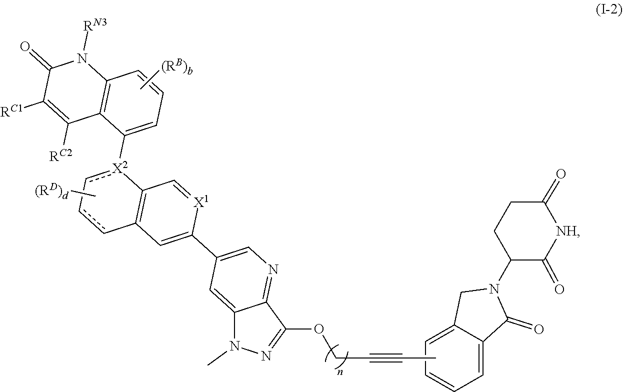

or a pharmaceutically acceptable salt, solvate, or stereoisomer thereof.

In certain embodiments, the compound is of Formula I-2-b:

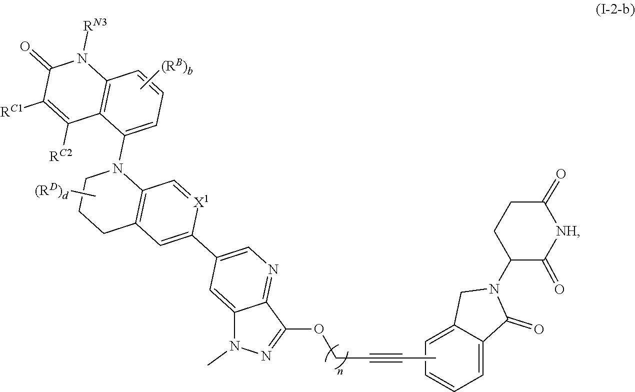

or a pharmaceutically acceptable salt, solvate, or stereoisomer thereof.

In certain embodiments, the compound is of Formula I-A:

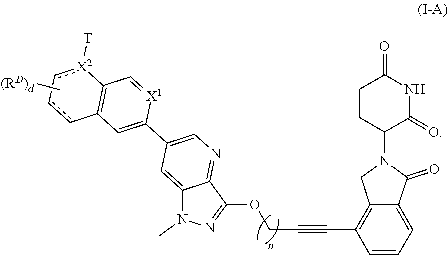

or a pharmaceutically acceptable salt, solvate, or stereoisomer thereof.

In certain embodiments, the compound is of Formula I-A-a:

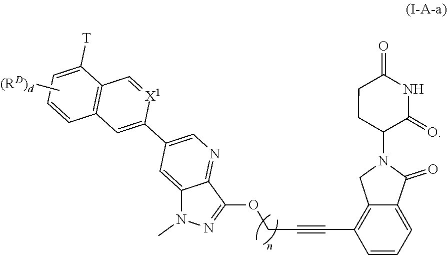

or a pharmaceutically acceptable salt, solvate, or stereoisomer thereof.

In certain embodiments, the compound is of Formula I-A-b:

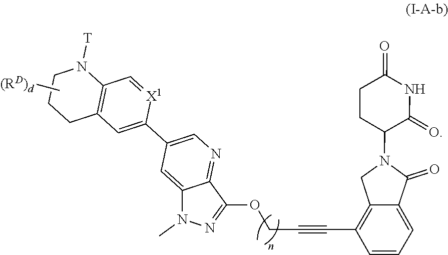

or a pharmaceutically acceptable salt, solvate, or stereoisomer thereof.

In certain embodiments, the compound is of Formula I-A-1:

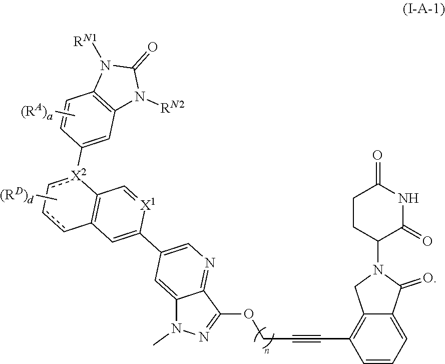

or a pharmaceutically acceptable salt, solvate, or stereoisomer thereof.

In certain embodiments, the compound is of Formula I-A-1-a:

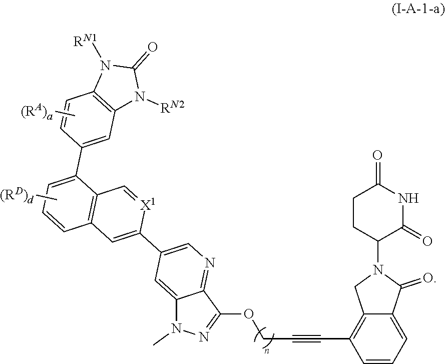

or a pharmaceutically acceptable salt, solvate, or stereoisomer thereof.

In certain embodiments, the compound is of Formula I-A-2:

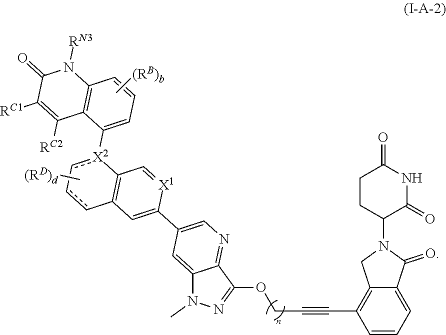

or a pharmaceutically acceptable salt, solvate, or stereoisomer thereof.

In certain embodiments, the compound is of Formula I-A-2-b:

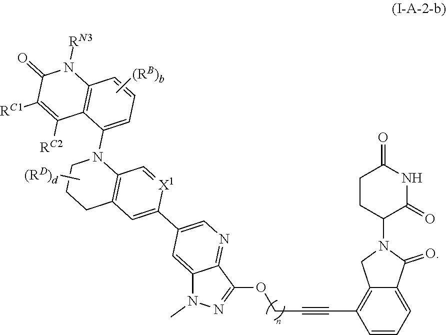

or a pharmaceutically acceptable salt, solvate, or stereoisomer thereof.

Embodiments of the variables in any of the Formulae described herein, e.g., Formulae A, I, I-a, I-b, I-1, I-1-a, I-2, I-2-b, I-A, I-A-a, I-A-b, I-A-1, I-A-1-a, I-A-2, I-A-2-b, T-1, T-1-a, T-2, T-2-a, C-1, C-1-i, C-1-i-a, C-1-i-b, C-1-i-c, C-1-i-a-1, C-1-i-a-2, C-1-i-a-3, C-1-i-a-4, C-1-i-a-i, C-1-i-a-ii, C-1-i-d, C-1-ii, C-1-ii-a, C-1-ii-b, C-1-iii, or C-1-i-a, as applicable, are described below. Any of the variables can be any moiety as described in the embodiments below. In addition, the combination of any moieties described for any of the variables, as applicable, with any moieties described for any of the remaining variables, is also contemplated.

### Variable T

In certain embodiments, T is T-1:

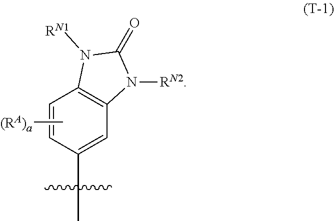

In certain embodiments, T is T-1-a:

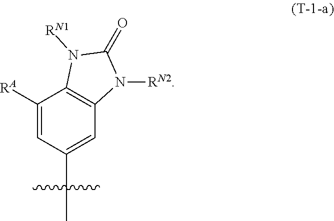

In certain embodiments, T is

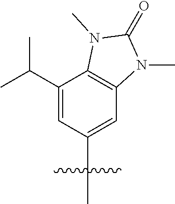 [SMILES: *c1cc(C(C)C)c2c(c1)n(C)c(=O)n2C]

In certain embodiments, T is T-2:

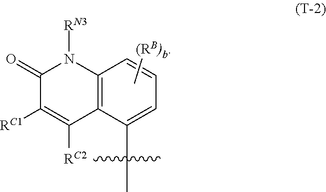

In certain embodiments, T is T-2-a:

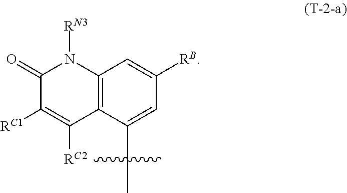

In certain embodiments, T is

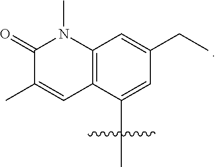 [SMILES: *c1cc(CC)cc2c1cc(C)c(=O)n2C]

In certain embodiments, R^{N1} is hydrogen.

In certain embodiments, R^{N1} is C₁-C₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)) optionally substituted with one or more halogen (e.g., —F, —Cl, —Br, or —I), —CN, —OH, or —NH₂.

In certain embodiments, R^{N1} is C₁-C₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)).

In certain embodiments, R^{N1} is C₁-C₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)) substituted with one or more halogen (e.g., —F, —Cl, —Br, or —I), —CN, —OH, or —NH₂.

In certain embodiments, R^{N1} is —CH₃.

In certain embodiments, R^{N2} is hydrogen.

In certain embodiments, R^{N2} is C₁-C₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)) optionally substituted with one or more halogen (e.g., —F, —Cl, —Br, or —I), —CN, —OH, or —NH₂.

In certain embodiments, R^{N2} is C₁-C₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)).

In certain embodiments, R^{N2} is C₁-C₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)) substituted with one or more halogen (e.g., —F, —Cl, —Br, or —I), —CN, —OH, or —NH₂.

In certain embodiments, R^{N2} is —CH₃.

In certain embodiments, R^{N1} and R^{N2} are each independently hydrogen.

In certain embodiments, R^{N1} and R^{N2} are each independently C₁-C₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)) optionally substituted with one or more halogen (e.g., —F, —Cl, —Br, or —I), —CN, —OH, or —NH₂.

In certain embodiments, R^{N1} and R^{N2} are each independently C₁-C₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)).

In certain embodiments, R^{N1} and R^{N2} are each independently C₁-C₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)) substituted with one or more halogen (e.g., —F, —Cl, —Br, or —I), —CN, —OH, or —NH₂.

In certain embodiments, R^{N1} and R^{N2} are each independently —CH₃.

In certain embodiments, R^{N3} is hydrogen.

In certain embodiments, R^{N3} is C₁-C₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)) optionally substituted with one or more halogen (e.g., —F, —Cl, —Br, or —I), —CN, —OH, or —NH₂.

In certain embodiments, R^{N3} is C₁-C₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)).

In certain embodiments, R^{N3} is C₁-C₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)) substituted with one or more halogen (e.g., —F, —Cl, —Br, or —I), —CN, —OH, or —NH₂.

In certain embodiments, R^{N3} is —CH₃.

In certain embodiments, R^{C1} is hydrogen.

In certain embodiments, Re is C₁-C₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)) optionally substituted with one or more halogen (e.g., —F, —Cl, —Br, or —I), —CN, —OH, or —NH₂.

In certain embodiments, Re is C₁-C₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)).

In certain embodiments, Re is C₁-C₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)) substituted with one or more halogen (e.g., —F, —Cl, —Br, or —I), —CN, —OH, or —NH₂.

In certain embodiments, R^{C1} is —CH₃.

In certain embodiments, R^{C2} is hydrogen.

In certain embodiments, R^{C2} is C₁-C₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)) optionally substituted with one or more halogen (e.g., —F, —Cl, —Br, or —I), —CN, —OH, or —NH₂.

In certain embodiments, R^{C2} is C₁-C₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)).

In certain embodiments, R^{C2} is C₁-C₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)) substituted with one or more halogen (e.g., —F, —Cl, —Br, or —I), —CN, —OH, or —NH₂.

In certain embodiments, at least one R^{A} independently is halogen (e.g., —F, —Cl, —Br, or —I).

In certain embodiments, at least one R^{A} independently is —CN.

In certain embodiments, at least one R^{A} independently is —OH.

In certain embodiments, at least one R^{A} independently is —NH₂.

In certain embodiments, at least one R^{A} independently is C₁-C₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)) optionally substituted with one or more halogen (e.g., —F, —Cl, —Br, or —I), —CN, —OH, or —NH₂.

In certain embodiments, at least one R^{A} independently is C₁-C₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)).

In certain embodiments, at least one R^{A} independently is C₁-C₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)) substituted with one or more halogen (e.g., —F, —Cl, —Br, or —I), —CN, —OH, or —NH₂.

In certain embodiments, at least one R^{A} independently is —CH(CH₃)₂.

In certain embodiments, each R^{A} independently is halogen (e.g., —F, —Cl, —Br, or —I).

In certain embodiments, each R^{A} independently is —CN.

In certain embodiments, each R^{A} independently is —OH.

In certain embodiments, each R^{A} independently is —NH₂.

In certain embodiments, each R^{A} independently is C₁-C₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)) optionally substituted with one or more halogen (e.g., —F, —Cl, —Br, or —I), —CN, —OH, or —NH₂.

In certain embodiments, each R^{A} independently is C₁-C₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)).

In certain embodiments, each R^{A} independently is C₁-C₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)) substituted with one or more halogen (e.g., —F, —Cl, —Br, or —I), —CN, —OH, or —NH₂.

In certain embodiments, each R^{A} independently is —CH(CH₃)₂.

In certain embodiments, at least one R^{B} independently is halogen (e.g., —F, —Cl, —Br, or —I).

In certain embodiments, at least one R^{B} independently is —CN.

In certain embodiments, at least one R^{B} independently is —OH.

In certain embodiments, at least one R^{B} independently is —NH₂.

In certain embodiments, at least one R^{B} independently is C₁-C₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)) optionally substituted with one or more halogen (e.g., —F, —Cl, —Br, or —I), —CN, —OH, or —NH₂.

In certain embodiments, at least one RB independently is C₁-C₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)).

In certain embodiments, at least one R^{B} independently is C₁-C₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)) substituted with one or more halogen (e.g., —F, —Cl, —Br, or —I), —CN, —OH, or —NH₂.

In certain embodiments, at least one R^{B} independently is —CH₂(CH₃).

In certain embodiments, each R^{B} independently is halogen (e.g., —F, —Cl, —Br, or —I).

In certain embodiments, each R^{B} independently is —CN.

In certain embodiments, each R^{B} independently is —OH.

In certain embodiments, each R^{B} independently is —NH₂.

In certain embodiments, each RB independently is C₁-C₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)) optionally substituted with one or more halogen (e.g., —F, —Cl, —Br, or —I), —CN, —OH, or —NH₂.

In certain embodiments, each RB independently is C₁-C₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)).

In certain embodiments, each RB independently is C₁-C₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)) substituted with one or more halogen (e.g., —F, —Cl, —Br, or —I), —CN, —OH, or —NH₂.

In certain embodiments, each R^{B} independently is —CH₂(CH₃).

In certain embodiments, a is 0. In certain embodiments, a is 1. In certain embodiments, a is 2. In certain embodiments, a is 3.

In certain embodiments, b is 0. In certain embodiments, b is 1. In certain embodiments, b is 2. In certain embodiments, b is 3.

Variables X¹, X², n, R^{D}, and d

In certain embodiments, at least one [symbol] is a single bond.

In certain embodiments, at least one [symbol] is a double bond.

In certain embodiments, each [symbol] is a single bond.

In certain embodiments, each [symbol] is a double bond.

In certain embodiments, X¹ is N.

In certain embodiments, X¹ is CR^{X1}.

In certain embodiments, X¹ is C(CHF₂).

In certain embodiments, X² is N.

In certain embodiments, X² is C.

In certain embodiments, R^{X1} is hydrogen.

In certain embodiments, R^{X1} is C₁-C₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)) optionally substituted with one or more halogen (e.g., —F, —Cl, —Br, or —I), —CN, —OH, or —NH₂.

In certain embodiments, R^{X1} is C₁-C₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)).

In certain embodiments, R^{X1} is C₁-C₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)) substituted with one or more halogen (e.g., —F, —Cl, —Br, or —I), —CN, —OH, or —NH₂.

In certain embodiments, R^{X1} is —CHF₂.

In certain embodiments, at least one R^{D} independently is halogen (e.g., —F, —Cl, —Br, or —I).

In certain embodiments, at least one R^{D} independently is —CN.

In certain embodiments, at least one R^{D} independently is —OH.

In certain embodiments, at least one R^{D} independently is —NH₂.

In certain embodiments, at least one R^{D} independently is C₁-C₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)) optionally substituted with one or more halogen (e.g., —F, —Cl, —Br, or —I), —CN, —OH, or —NH₂.

In certain embodiments, at least one R^{D} independently is C₁-C₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)).

In certain embodiments, at least one R^{D} independently is C₁-C₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)) substituted with one or more halogen (e.g., —F, —Cl, —Br, or —I), —CN, —OH, or —NH₂.

In certain embodiments, each R^{D} independently is halogen (e.g., —F, —Cl, —Br, or —I).

In certain embodiments, each R^{D} independently is —CN.

In certain embodiments, each R^{D} independently is —OH.

In certain embodiments, each R^{D} independently is —NH₂.

In certain embodiments, each R^{D} independently is C₁-C₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)) optionally substituted with one or more halogen (e.g., —F, —Cl, —Br, or —I), —CN, —OH, or —NH₂.

In certain embodiments, each R^{D} independently is C₁-C₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)).

In certain embodiments, each R^{D} independently is C₁-C₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)) substituted with one or more halogen (e.g., —F, —Cl, —Br, or —I), —CN, —OH, or —NH₂.

In certain embodiments, d is 0. In certain embodiments, d is 1. In certain embodiments, d is 2. In certain embodiments, d is 3. In certain embodiments, d is 4. In certain embodiments, d is 5.

In certain embodiments, n is an integer selected from 1 to 3.

In certain embodiments, n is 1. In certain embodiments, n is 2. In certain embodiments, n is 3. In certain embodiments, n is 4. In certain embodiments, n is 5.

In certain embodiments,

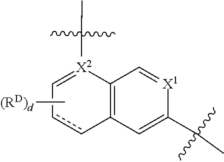

is

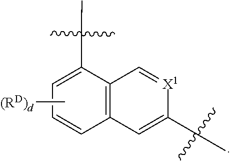

In certain embodiments,

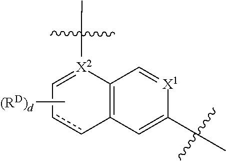

is

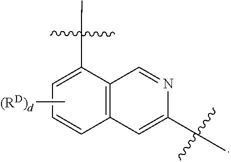

In certain embodiments,

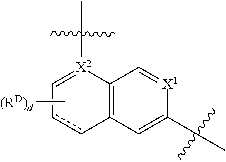

is

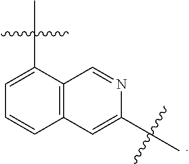 [SMILES: *c1cc2cccc(*)c2cn1]

In certain embodiments,

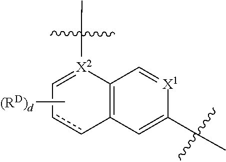

is

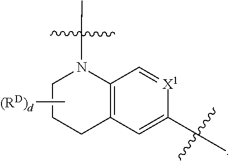

In certain embodiments,

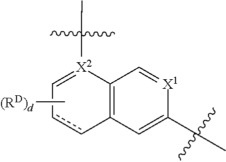

is

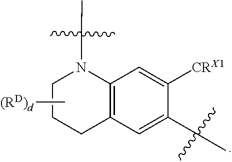

In certain embodiments,

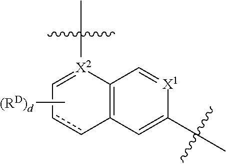

is

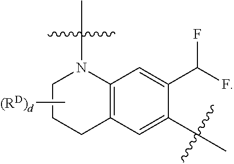

In certain embodiments,

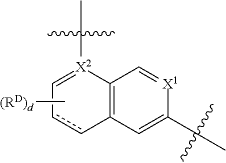

is

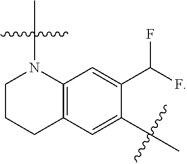 [SMILES: *c1cc2c(cc1C(F)F)N(*)CCC2]

### Variable C

In some embodiments, C is as described in PCT Application No. PCT/US2023/084247 (incorporated herein by reference).

In some embodiments C is C-1:

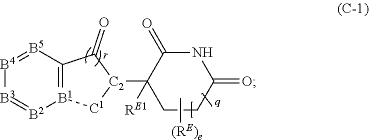

B² is CR^{B2} or N;
B³ is CR^{B3} or N;
B⁴ is CR^{B4} or N;
B⁵ is CR^{B5} or N;
R^{B2}, R^{B3}, R^{B4}, and R^{B5} are independently

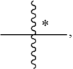

hydrogen, halogen, —CN, —NO₂, —OH, —NH₂, C₁-C₆ alkyl, C₁-C₆ alkoxy, C₁-C₆ alkylamino, C₂-C₆ alkenyl, C₂-C₆ alkynyl, C₃-C₁₂ carbocyclyl, 3- to 12-membered heterocyclyl, C₆-C₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
wherein

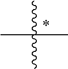

denotes attachment to the rest of the compound, and only one of R^{B2}, R^{B3}, R^{B4}, and R^{B5} is

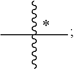

or
either R^{B2} and R^{B3}, R^{B3} and R^{B4}, or R^{B4} and R^{B5} form Ring A attached to the rest of the compound, wherein Ring A is optionally substituted C₃-C₁₂ carbocycle, 3- to 12-membered heterocycle, C₆-C₁₀ aryl, or 5- to 10-membered heteroaryl;
- - - denotes an optional covalent bond between B¹ and C¹;
i) when the bond between B¹ and C¹ is present:
r is 0 or 1;
C² is N;
B¹ is C; and
C¹ is —C(R^{C1i})₂—, —C(═O)—, ^{#}—C═N(R^{C1ii})—, or ^{#}—C(═O)N(R^{C1ii})—, wherein ^{#} denotes attachment to C²;
each R^{C1i} is independently hydrogen, halogen, —CN, —NO₂, —OH, —NH₂, C₁-C₆ alkyl, C₁-C₆ alkoxy, C₁-C₆ alkylamino, C₂-C₆ alkenyl, C₂-C₆ alkynyl, C₃-C₁₂ carbocyclyl, 3- to 12-membered heterocyclyl, C₆-C₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}; or
two R^{C1i}, together with the carbon atom to which they are attached, form C₃-C₆ carbocycle or 3- to 6-membered heterocycle, wherein the carbocycle or heterocycle optionally substituted with one or more R^{u}; and
R^{C1ii} is hydrogen or C₁-C₆ alkyl, wherein the C₁-C₆ alkyl is optionally substituted with one or more R^{u};
ii) when the bond between B¹ and C¹ is absent:
r is 0 or 1;
C² is absent, O, or N; wherein when C² is O, then C¹ is absent; when C² is N, then C¹ is hydrogen, C₁-C₆ alkyl, C₃-C₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl, or heterocyclyl is optionally substituted with one or more R^{u};
B¹ is N or CR^{B1}; and
R^{B1} is hydrogen, halogen, —CN, —NO₂, —OH, —NH₂, C₁-C₆ alkyl, C₁-C₆ alkoxy, C₁-C₆ alkylamino, C₂-C₆ alkenyl, C₂-C₆ alkynyl, C₃-C₁₂ carbocyclyl, 3- to 12-membered heterocyclyl, C₆-C₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —N S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
R^{E1} is hydrogen, deuterium, or C₁-C₆ alkyl, wherein the C₁-C₆ alkyl is optionally substituted with one or more R^{u};
each R^{E} is independently halogen, —CN, —NO₂, —OH, —NH₂, C₁-C₆ alkyl, C₁-C₆ alkoxy, C₁-C₆ alkylamino, C₂-C₆ alkenyl, C₂-C₆ alkynyl, C₃-C₁₂ carbocyclyl, 3- to 12-membered heterocyclyl, C₆-C₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
e is an integer from 0 to 6, as valency permits;
q is an integer from 0 to 2;
each R^{u} is independently oxo, halogen, —CN, —NO₂, —OH, —NH₂, C₁-C₆ alkyl, C₁-C₆ alkoxy, C₁-C₆ alkylamino, C₂-C₆ alkenyl, C₂-C₆ alkynyl, C₃-C₁₂ carbocyclyl, 3- to 12-membered heterocyclyl, C₆-C₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —N S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}; wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁-C₆ alkyl, C₁-C₆ alkoxy, C₁-C₆ alkylamino, C₂-C₆ alkenyl, C₂-C₆ alkynyl, C₃-C₆ carbocyclyl, and 3- to 6-membered heterocyclyl; or
two R^{u}, together with the one or more intervening atoms, form C₆-C₁₀ aryl, 5- to 10-membered heteroaryl, C₃-C₁₂ carbocyclyl or 3- to 12-membered heterocyclyl;
each R^{a} is independently C₁-C₆ alkyl, C₂-C₆ alkenyl, C₂-C₆ alkynyl, C₃-C₁₂ carbocyclyl, 3- to 12-membered heterocyclyl, C₆-C₁₀ aryl, or 5- to 10-membered heteroaryl;
each R^{b} is independently hydrogen, C₁-C₆ alkyl, C₂-C₆ alkenyl, C₂-C₆ alkynyl, C₃-C₁₂ carbocyclyl, 3- to 12-membered heterocyclyl, C₆-C₁₀ aryl, or 5- to 10-membered heteroaryl; and
each R^{c} and R^{d} is independently hydrogen, C₁-C₆ alkyl, C₂-C₆ alkenyl, C₂-C₆ alkynyl, C₃-C₁₂ carbocyclyl, 3- to 12-membered heterocyclyl, C₆-C₁₀ aryl, or 5- to 10-membered heteroaryl; or
R^{c} and R^{d}, together with the nitrogen atom to which they are attached, form 3- to 12-membered heterocyclyl,
wherein each occurrence of R^{a}, R^{b}, R^{c}, and R^{d} is independently and optionally substituted with one or more R^{z}; and
each R^{z} is independently oxo, halogen, —CN, —NO₂, —OH, —NH₂, C₁-C₆ alkyl, C₁-C₆ alkoxy, C₁-C₆ alkylamino, C₂-C₆ alkenyl, C₂-C₆ alkynyl, C₃-C₆ carbocyclyl, or 3- to 6-membered heterocyclyl.

In certain embodiments, C is C-1-i:

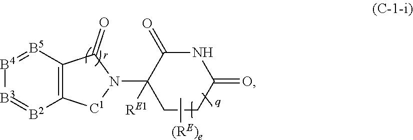

wherein R^{B2}, R^{B4}, R^{B3}, or R^{B5} is

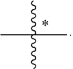

In certain embodiments, C is C-1-i-a, C-1-i-b, or C-1-i-c:

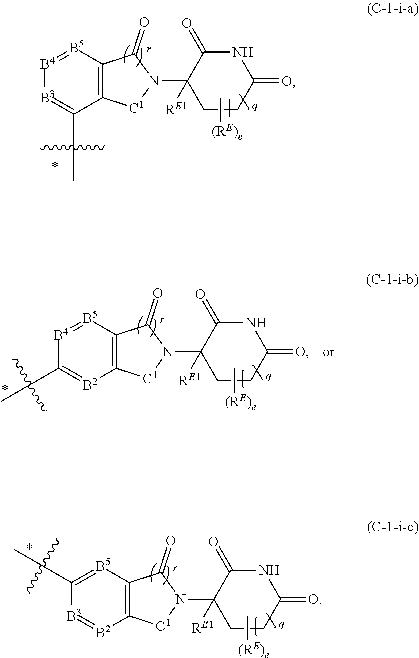

In certain embodiments, C is C-1-i-a-i, C-1-i-a-2, C-1-i-a-3, or C-1-i-a-4:

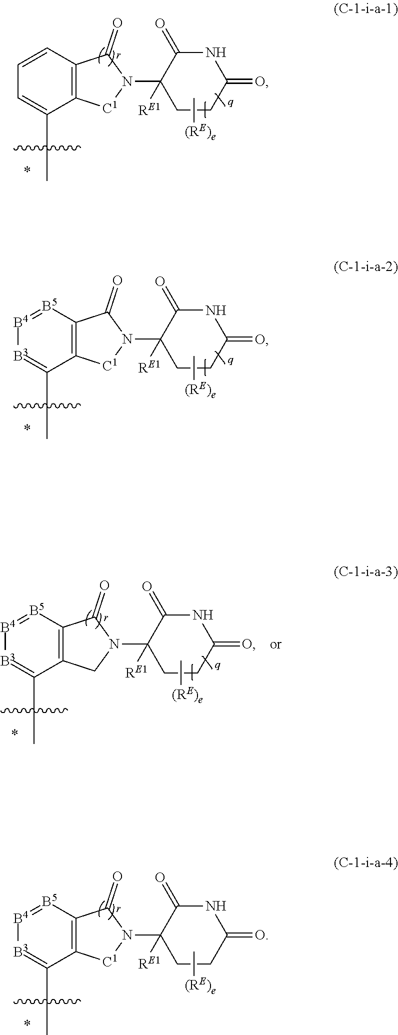

In certain embodiments, C is C-1-i-a-i or C-1-i-a-ii:

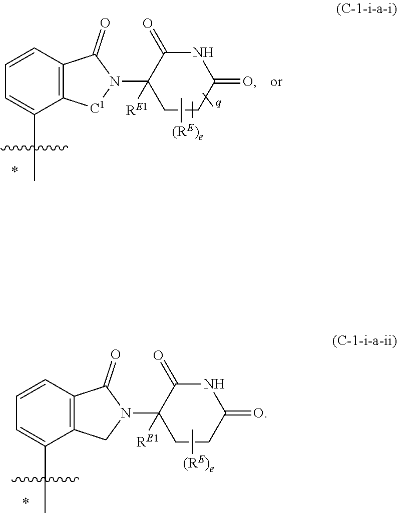

In certain embodiments, C is

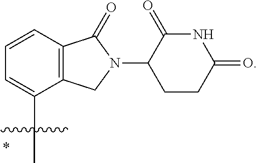 [SMILES: *c1cccc2c1CN(C1CCC(=O)NC1=O)C2=O]

In certain embodiments, C is C-1-i-d:

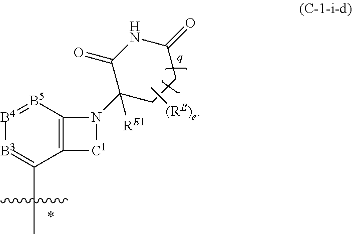

In certain embodiments, C is C-1-ii:

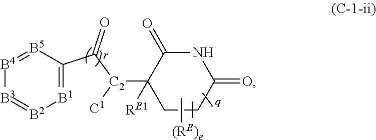

wherein R^{B2}, R^{B4}, R^{B3}, or R^{B5} is

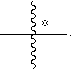

In certain embodiments, C is C-1-ii-a or C-1-ii-b:

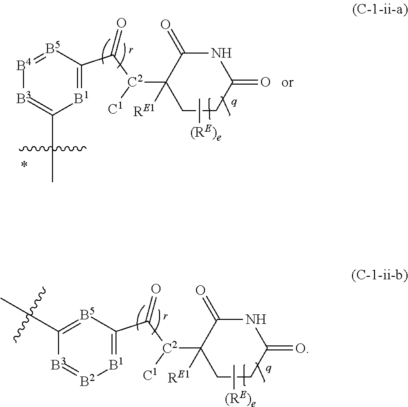

In certain embodiments, C is C-1-iii:

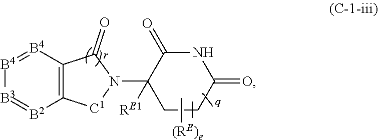

wherein R^{B2} and R^{B3}, R^{B3} and R^{B4}, or R^{B4} and R^{B5} form Ring A attached to the rest of the compound, wherein Ring A is optionally substituted 3- to 12-membered heterocycle, C₆-C₁₀ aryl, or 5- to 10-membered heteroaryl.

In certain embodiments C is C-1-iii-a:

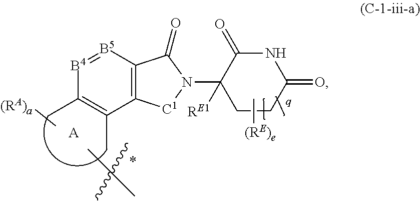

wherein:
Ring A is 5- to 7-membered heterocycle;
each R^{A} is independently oxo, halogen, —CN, —NO₂, —OH, —NH₂, C₁-C₆ alkyl, C₁-C₆ alkoxy, C₁-C₆ alkylamino, C₂-C₆ alkenyl, C₂-C₆ alkynyl, C₃-C₁₂ carbocyclyl, 3- to 12-membered heterocyclyl, C₆-C₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —O(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; and
a is an integer selected from 0 to 10, as valency permits.

In certain embodiments, B² is CR^{B2}. In certain embodiments, B² is N.

In certain embodiments, B³ is CR^{B3}. In certain embodiments, B³ is N.

In certain embodiments, B⁴ is CR^{B4}. In certain embodiments, B⁴ is N.

In certain embodiments, B⁵ is CR^{B5}. In certain embodiments, B⁵ is N.

In certain embodiments, one of B², B³, B⁴, and B⁵ is N. In certain embodiments, two of B², B³, B⁴, and B⁵ are N. In certain embodiments, three of B², B³, B⁴, and B⁵ are N. In certain embodiments, four of B², B³, B⁴, and B⁵ are N.

In certain embodiments, R^{B2}, R^{B3}, R^{B4}, and R^{B5} are independently

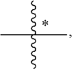

hydrogen, halogen (e.g., —F, —Cl, —Br, or —I), —CN, —NO₂, —OH, —NH₂, C₁-C₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁-C₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁-C₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-1-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂-C₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂-C₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃-C₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆-C₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —N S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}.

In certain embodiments, R^{B2}, R^{B3}, R^{B4}, and R^{B5} are independently

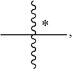

hydrogen, halogen, —CN, —NO₂, —OH, —NH₂, C₁-C₆ alkyl, C₁-C₆ alkoxy, C₁-C₆ alkylamino, C₂-C₆ alkenyl, C₂-C₆ alkynyl, C₃-C₁₂ carbocyclyl, 3- to 12-membered heterocyclyl, C₆-C₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, R^{B2}, R^{B3}, R^{B4}, and R^{B5} are independently

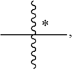

hydrogen, halogen, —CN, —NO₂, —OH, —NH₂, C₁-C₆ alkyl, C₁-C₆ alkoxy, C₁-C₆ alkylamino, C₂-C₆ alkenyl, C₂-C₆ alkynyl, C₃-C₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, R^{B2}, R^{B3}, R^{B4}, and R^{B5} are independently

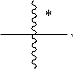

hydrogen, halogen, —CN, —NO₂, —OH, —NH₂, C₁-C₆ alkyl, C₁-C₆ alkoxy, C₁-C₆ alkylamino, C₂-C₆ alkenyl, C₂-C₆ alkynyl, C₃-C₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u}.

In certain embodiments, R^{B2}, R^{B3}, R^{B4}, and R^{B5} are independently

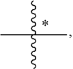

hydrogen, halogen, —CN, —NO₂, —OH, —NH₂, C₁-C₆ alkyl, C₁-C₆ alkoxy, C₁-C₆ alkylamino, C₃-C₆ carbocyclyl, or 3- to 6-membered heterocyclyl, wherein the alkyl, alkoxy, alkylamino, carbocyclyl, or heterocyclyl is optionally substituted with one or more R^{u}.

In certain embodiments,

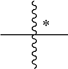

denotes attachment to the rest of the compound, and only one of R^{B2}, R^{B3}, R^{B4}, and R^{B5} is

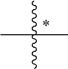

In certain embodiments, R^{B2} and R^{B3}, R^{B3} and R^{B4}, or R^{B4} and R^{B5} form Ring A attached to the rest of the compound, wherein Ring A is optionally substituted C₃-C₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆-C₁₀ aryl (e.g., phenyl or naphthyl), or 5- to 10-membered heteroaryl (e.g., heteroaryl comprising one or two 5- or 6-membered rings and 1-5 heteroatoms selected from N, O, and S).

In certain embodiments, - - - denotes an optional covalent bond between B¹ and C¹. In certain embodiments, the bond between B¹ and C¹ is present. In certain embodiments, the bond between B¹ and C¹ is absent.

In certain embodiments, r is 0. In certain embodiments, r is 1.

In certain embodiments, C¹ is —C(R^{C1i})₂—.

In certain embodiments, C¹ is —C(═O)—.

In certain embodiments, C¹ is ^{#}—C═N(R^{C1ii})—.

In certain embodiments, C¹ is ^{#}—C(═O)N(R^{C1ii})—, wherein ^{#} denotes attachment to C².

In certain embodiments, each R^{C1i} is independently hydrogen, halogen (e.g., —F, —Cl, —Br, or —I), —CN, —NO₂, —OH, —NH₂, C₁-C₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁-C₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁-C₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂-C₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂-C₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃-C₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆-C₁₀ aryl (e.g., phenyl or naphthyl), or 5- to 10-membered heteroaryl (e.g., heteroaryl comprising one or two 5- or 6-membered rings and 1-5 heteroatoms selected from N, O, and S), wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{C1i} is independently hydrogen, halogen, —CN, —NO₂, —OH, —NH₂, C₁-C₆ alkyl, C₁-C₆ alkoxy, C₁-C₆ alkylamino, C₂-C₆ alkenyl, C₂-C₆ alkynyl, C₃-C₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{C1i} is independently hydrogen, halogen, —CN, —NO₂, —OH, —NH₂, C₁-C₆ alkyl, C₁-C₆ alkoxy, C₁-C₆ alkylamino, C₂-C₆ alkenyl, C₂-C₆ alkynyl, C₃-C₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u}.

In certain embodiments, each R^{C1i} is independently hydrogen, halogen, —CN, —NO₂, —OH, —NH₂, C₁-C₆ alkyl, C₁-C₆ alkoxy, C₁-C₆ alkylamino, C₃-C₆ carbocyclyl, or 3- to 6-membered heterocyclyl, wherein the alkyl, alkoxy, alkylamino, carbocyclyl, or heterocyclyl is optionally substituted with one or more R^{u}.

In certain embodiments, two R^{C1i}, together with the carbon atom to which they are attached, form C₃-C₆ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), or cyclohexadienyl (C₆)) or 3- to 6-membered heterocyclyl (e.g., heterocyclyl comprising one 3- to 6-membered ring and 1-3 heteroatoms selected from N, O, and S), wherein the carbocycle or heterocycle optionally substituted with one or more R^{u}.

In certain embodiments, R^{C1ii} is hydrogen or C₁-C₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)) optionally substituted with one or more R^{u}.

In certain embodiments, C¹ is absent.

In certain embodiments, C¹ is hydrogen, C₁-C₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₃-C₆ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), or cyclohexadienyl (C₆)), or 3- to 6-membered heterocyclyl (e.g., heterocyclyl comprising one 3- to 6-membered ring and 1-3 heteroatoms selected from N, O, and S), —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl, or heterocyclyl is optionally substituted with one or more R^{u}.

In certain embodiments, C² is absent.

In certain embodiments, C² is O.

In certain embodiments, C² is N.

In certain embodiments, B¹ is N.

In certain embodiments, B¹ is CR^{B1}.

In certain embodiments, R^{B1} is hydrogen, halogen (e.g., —F, —Cl, —Br, or —I), —CN, —NO₂, —OH, —NH₂, C₁-C₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁-C₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁-C₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, 1-butylpentylamino, n-butylhexylamino, i-butylhexylamino, s-butylhexylamino, t-butylhexylamino, or pentylhexylamino), C₂-C₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂-C₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃-C₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆-C₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}.

In certain embodiments, R^{B1} is hydrogen, halogen, —CN, —NO₂, —OH, —NH₂, C₁-C₆ alkyl, C₁-C₆ alkoxy, C₁-C₆ alkylamino, C₂-C₆ alkenyl, C₂-C₆ alkynyl, C₃-C₁₂ carbocyclyl, 3- to 12-membered heterocyclyl, C₆-C₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, R^{B1} is hydrogen, halogen, —CN, —NO₂, —OH, —NH₂, C₁-C₆ alkyl, C₁-C₆ alkoxy, C₁-C₆ alkylamino, C₂-C₆ alkenyl, C₂-C₆ alkynyl, C₃-C₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, R^{B1} is hydrogen, halogen, —CN, —NO₂, —OH, —NH₂, C₁-C₆ alkyl, C₁-C₆ alkoxy, C₁-C₆ alkylamino, C₂-C₆ alkenyl, C₂-C₆ alkynyl, C₃-C₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u}.

In certain embodiments, R^{B1} is hydrogen, halogen, —CN, —NO₂, —OH, —NH₂, C₁-C₆ alkyl, C₁-C₆ alkoxy, C₁-C₆ alkylamino, C₃-C₆ carbocyclyl, or 3- to 6-membered heterocyclyl, wherein the alkyl, alkoxy, alkylamino, carbocyclyl, or heterocyclyl is optionally substituted with one or more R^{u}.

In certain embodiments, R^{E1} is hydrogen.

In certain embodiments, R^{E1} is deuterium.

In certain embodiments, R^{E1} is C₁-C₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)) optionally substituted with one or more R^{u}.

In certain embodiments, each R^{E} is independently oxo, hydrogen, halogen (e.g., —F, —Cl, —Br, or —I), —CN, —NO₂, —OH, —NH₂, C₁-C₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁-C₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁-C₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂-C₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂-C₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃-C₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆-C₁₀ aryl (e.g., phenyl or naphthyl), or 5- to 10-membered heteroaryl (e.g., heteroaryl comprising one or two 5- or 6-membered rings and 1-5 heteroatoms selected from N, O, and S), wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{E} is independently oxo, hydrogen, halogen, —CN, —NO₂, —OH, —NH₂, C₁-C₆ alkyl, C₁-C₆ alkoxy, C₁-C₆ alkylamino, C₂-C₆ alkenyl, C₂-C₆ alkynyl, C₃-C₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{E} is independently oxo, hydrogen, halogen, —CN, —NO₂, —OH, —NH₂, C₁-C₆ alkyl, C₁-C₆ alkoxy, C₁-C₆ alkylamino, C₂-C₆ alkenyl, C₂-C₆ alkynyl, C₃-C₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u}.

In certain embodiments, each R^{E} is independently oxo, hydrogen, halogen, —CN, —NO₂, —OH, —NH₂, C₁-C₆ alkyl, C₁-C₆ alkoxy, C₁-C₆ alkylamino, C₃-C₆ carbocyclyl, or 3- to 6-membered heterocyclyl, wherein the alkyl, alkoxy, alkylamino, carbocyclyl, or heterocyclyl is optionally substituted with one or more R^{u}.

In certain embodiments, e is an integer from 0 to 6, as valency permits. In certain embodiments, e is 1. In certain embodiments, e is 2. In certain embodiments, e is 3, as valency permits. In certain embodiments, e is 4, as valency permits. In certain embodiments, e is 5, as valency permits. In certain embodiments, e is 6.

In certain embodiments, q is an integer from 0 to 2. In certain embodiments, q is 0. In certain embodiments, q is 1. In certain embodiments, q is 2.

In certain embodiments, each R is independently C₁-C₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₂-C₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆), C₂-C₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃-C₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆-C₁₀ aryl (e.g., phenyl or naphthyl), or 5- to 10-membered heteroaryl (e.g., heteroaryl comprising one or two 5- or 6-membered rings and 1-5 heteroatoms selected from N, O, and S), wherein the alkyl, alkenyl, alkynyl, carbocyclyl, heterocyclyl, aryl, or heteroaryl is optionally substituted with one or more R^{u}.

In certain embodiments, each R^{a} is independently C₁-C₆ alkyl, C₂-C₆ alkenyl, C₂-C₆ alkynyl, C₃-C₆ carbocyclyl, 3- to 6-membered heterocyclyl, C₆ aryl, or 5- to 6-membered heteroaryl.

In certain embodiments, each R^{a} is independently C₁-C₆ alkyl, C₂-C₆ alkenyl, C₂-C₆ alkynyl, C₃-C₆ carbocyclyl, or 3- to 6-membered heterocyclyl.

In certain embodiments, each R is independently C₁-C₆ alkyl, C₃-C₆ carbocyclyl, or 3- to 6-membered heterocyclyl, wherein the alkyl, carbocyclyl, or heterocyclyl is optionally substituted with one or more R^{u}.

In certain embodiments, each R^{b} is independently hydrogen, C₁-C₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₂-C₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆), C₂-C₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃-C₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆-C₁₀ aryl (e.g., phenyl or naphthyl), or 5- to 10-membered heteroaryl (e.g., heteroaryl comprising one or two 5- or 6-membered rings and 1-5 heteroatoms selected from N, O, and S), wherein the alkyl, alkenyl, alkynyl, carbocyclyl, heterocyclyl, aryl, or heteroaryl is optionally substituted with one or more R^{u}.

In certain embodiments, each R^{b} is independently hydrogen, C₁-C₆ alkyl, C₂-C₆ alkenyl, C₂-C₆ alkynyl, C₃-C₆ carbocyclyl, 3- to 6-membered heterocyclyl, C₆ aryl, or 5- to 6-membered heteroaryl.

In certain embodiments, each R^{b} is independently hydrogen, C₁-C₆ alkyl, C₂-C₆ alkenyl, C₂-C₆ alkynyl, C₃-C₆ carbocyclyl, or 3- to 6-membered heterocyclyl.

In certain embodiments, each R^{b} is independently hydrogen, C₁-C₆ alkyl, C₃-C₆ carbocyclyl, or 3- to 6-membered heterocyclyl, or C₂-C₆ alkynyl, wherein the alkyl, carbocyclyl, or heterocyclyl is optionally substituted with one or more R^{u}.

In certain embodiments, each R^{c} and each R^{d} is independently hydrogen, C₁-C₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₂-C₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆), C₂-C₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃-C₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆-C₁₀ aryl (e.g., phenyl or naphthyl), or 5- to 10-membered heteroaryl (e.g., heteroaryl comprising one or two 5- or 6-membered rings and 1-5 heteroatoms selected from N, O, and S), wherein the alkyl, alkenyl, alkynyl, carbocyclyl, heterocyclyl, aryl, or heteroaryl is optionally substituted with one or more R^{u}.

In certain embodiments, each R^{c} and each R^{d} is independently hydrogen, C₁-C₆ alkyl, C₃-C₆ carbocyclyl, or 3- to 6-membered heterocyclyl, wherein the alkyl, carbocyclyl, or heterocyclyl is optionally substituted with one or more R^{u}.

In certain embodiments, R^{c} and R^{d}, together with the nitrogen atom to which they are attached, form 3- to 12-membered heterocyclyl (e.g., heterocyclyl comprising one or two 3- to 8-membered rings and 1-5 heteroatoms selected from N, O, and S), wherein the heterocyclyl is optionally substituted with one or more R^{u}.

In certain embodiments, R^{a}, R^{b}, R^{c}, and R^{d} is independently and optionally substituted with one or more R^{z}.

In certain embodiments, R^{z} is independently oxo, halogen, —CN, —NO₂, —OH, —NH₂, C₁-C₆ alkyl, C₁-C₆ alkoxy, C₁-C₆ alkylamino, C₂-C₆ alkenyl, C₂-C₆ alkynyl, C₃-C₆ carbocyclyl, or 3- to 6-membered heterocyclyl.

In certain embodiments, each R^{u} is independently oxo, halogen, —CN, —NO₂, —OH, —NH₂, C₁-C₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁-C₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁-C₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-i-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂-C₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂-C₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃-C₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆-C₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}; wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁-C₆ alkyl, C₁-C₆ alkoxy, C₁-C₆ alkylamino, C₂-C₆ alkenyl, C₂-C₆ alkynyl, C₃-C₆ carbocyclyl, and 3- to 6-membered heterocyclyl.

In certain embodiments, each R^{u} is independently oxo, halogen, —CN, —NO₂, —OH, —NH₂, C₁-C₆ alkyl, C₁-C₆ alkoxy, C₁-C₆ alkylamino, C₂-C₆ alkenyl, C₂-C₆ alkynyl, C₃-C₁₂ carbocyclyl, 3- to 12-membered heterocyclyl, C₆-C₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁-C₆ alkyl, C₁-C₆ alkoxy, C₁-C₆ alkylamino, C₂-C₆ alkenyl, C₂-C₆ alkynyl, C₃-C₆ carbocyclyl, and 3- to 6-membered heterocyclyl.

In certain embodiments, each R^{u} is independently oxo, halogen, —CN, —NO₂, —OH, —NH₂, C₁-C₆ alkyl, C₁-C₆ alkoxy, C₁-C₆ alkylamino, C₂-C₆ alkenyl, C₂-C₆ alkynyl, C₃-C₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁-C₆ alkyl, C₁-C₆ alkoxy, C₁-C₆ alkylamino, C₂-C₆ alkenyl, C₂-C₆ alkynyl, C₃-C₆ carbocyclyl, and 3- to 6-membered heterocyclyl.

In certain embodiments, each R^{u} is independently oxo, halogen, —CN, —NO₂, —OH, —NH₂, C₁-C₆ alkyl, C₁-C₆ alkoxy, C₁-C₆ alkylamino, C₂-C₆ alkenyl, C₂-C₆ alkynyl, C₃-C₆ carbocyclyl, or 3- to 6-membered heterocyclyl, wherein the alkyl, alkoxy, alkylamino, alkenyl, alkynyl, carbocyclyl or heterocyclyl is optionally substituted with one or more substituents selected from oxo, halogen, —CN, —NO₂, —OH, —NH₂, C₁-C₆ alkyl, C₁-C₆ alkoxy, C₁-C₆ alkylamino, C₂-C₆ alkenyl, C₂-C₆ alkynyl, C₃-C₆ carbocyclyl, and 3- to 6-membered heterocyclyl.

In certain embodiments, each R^{u} is independently oxo, halogen, —CN, —NO₂, —OH, —NH₂, C₁-C₆ alkyl, C₁-C₆ alkoxy, C₁-C₆ alkylamino, C₃-C₆ carbocyclyl, or 3- to 6-membered heterocyclyl, wherein the alkyl, alkoxy, alkylamino, carbocyclyl or heterocyclyl is optionally substituted with one or more substituents selected from oxo, halogen, —CN, —NO₂, —OH, —NH₂, C₁-C₆ alkyl, C₁-C₆ alkoxy, C₁-C₆ alkylamino, C₂-C₆ alkenyl, C₂-C₆ alkynyl, C₃-C₆ carbocyclyl, and 3- to 6-membered heterocyclyl.

In certain embodiments, two R^{u}, together with the carbon atom(s) to which they are attached, form C₃-C₆ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), or cyclohexadienyl (C₆)) or 3- to 6-membered heterocyclyl (e.g., heterocyclyl comprising one 3- to 6-membered ring and 1-3 heteroatoms selected from N, O, and S).

In certain embodiments, two geminal RU, together with the carbon atom to which they are attached, form C₃-C₆ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), or cyclohexadienyl (C₆)) or 3- to 6-membered heterocyclyl (e.g., heterocyclyl comprising one 3- to 6-membered ring and 1-3 heteroatoms selected from N, O, and S).

When a range of values is listed, each discrete value and sub-range within the range are also contemplated. For example, “C₁-C₆ alkyl” is intended to encompass, C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl.

In certain embodiments, the compound is selected from the compounds in Table 1 and pharmaceutically acceptable salts, solvates, and stereoisomers thereof.

In certain embodiments, the compound is selected from the compounds in Table 1 and pharmaceutically acceptable salts thereof.

In certain embodiments, the compound is selected from the compounds in Table 1 and solvates thereof.

In certain embodiments, the compound is selected from the compounds in Table 1 and stereoisomers thereof.

In certain embodiments, the compound is selected from the compounds in Table 1.

TABLE 1

| Com- | | |
|---|---|---|
| pound | | |
| No. | Structure | Chemical Name |
| 1 | 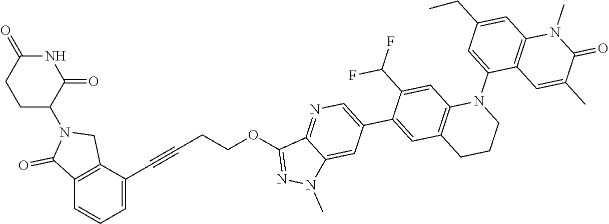 [SMILES: CCc1cc(N2CCCc3cc(-c4cnc5c(OCCC#Cc6cccc7c6CN(C6CCC(=O)NC6=O)C7=O)nn(C)c5c4)c(C(F)F)cc32)c2cc(C)c(=O)n(C)c2c1] | 3-(4-(4-((6-(7- (difluoromethyl)-7′-ethyl-1′,3′- dimethyl-2′-oxo-1′,2,′3,4- tetrahydro-2H-[1,5′- biquinolin]-6-yl)-1-methyl-1H- pyrazolo[4,3-b]pyridin-3- yl)oxy)but-1-yn-1-yl)-1- oxoisoindolin-2-yl)piperidine- 2,6-dione |
| 2 | 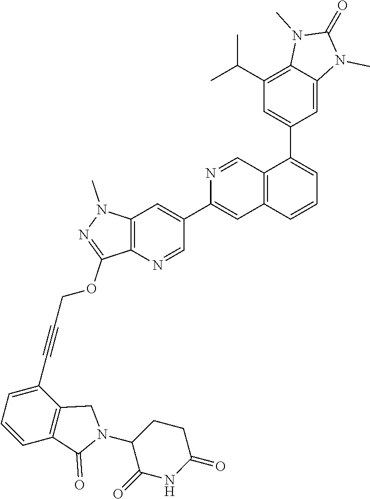 [SMILES: CC(C)c1cc(-c2cccc3cc(-c4cnc5c(OCC#Cc6cccc7c6CN(C6CCC(=O)NC6=O)C7=O)nn(C)c5c4)ncc23)cc2c1n(C)c(=O)n2C] | 3-(4-(3-((6-(8-(7-isopropyl- 1,3-dimethyl-2-oxo-2,3- dihydro-1H-benzo[d]imidazol- 5-yl)isoquinolin-3-yl)-1- methyl-1H-pyrazolo[4,3- b]pyridin-3-yl)oxy)prop-1-yn- 1-yl)-1-oxoisoindolin-2- yl)piperidine-2,6-dione |
| 3 | 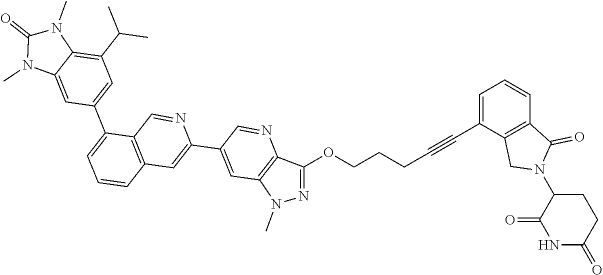 [SMILES: CC(C)c1cc(-c2cccc3cc(-c4cnc5c(OCCCC#Cc6cccc7c6CN(C6CCC(=O)NC6=O)C7=O)nn(C)c5c4)ncc23)cc2c1n(C)c(=O)n2C] | 3-(4-(5-((6-(8-(7-isopropyl- 1,3-dimethyl-2-oxo-2,3- dihydro-1H-benzo[d]imidazol- 5-yl)isoquinolin-3-yl)-1- methyl-1H-pyrazolo[4,3- b]pyridin-3-yl)oxy)pent-1-yn- 1-yl)-1-oxoisoindolin-2- yl)piperidine-2,6-dione |

The compounds of the present disclosure possess advantageous characteristics, as compared to other p300 degraders. For example, the compounds of the present disclosure may potentially show selectivity for p300 over CBP, display more potent degradation activity against p300, more favorable pharmacokinetic properties (e.g., as measured by Cₘₐₓ, Tₘₐₓ, and/or AUC), and/or less interaction with other cellular targets (e.g., hepatic cellular transporter such as OATP1B1) and accordingly improved safety (e.g., drug-drug interaction). In certain embodiments, a compound disclosed herein shows selective degradation of p300 over CBP when a compound has a DC₅₀ for p300 that is lower than its DC₅₀ for CBP. In certain embodiments, a compound disclosed herein shows selectivity when a compound has a Dₘₐₓ for p300 that is greater than its Dₘₐₓ for CBP. In certain embodiments, a compound disclosed herein shows selectivity through a combination of both lower DC₅₀ and greater Dₘₐₓ for p300, as compared to those for CBP. In certain embodiments, a compound disclosed herein shows selectivity when a compound has a DC₅₀ for p300 at least 10-fold lower than its DC₅₀ for CBP and/or a value of Dₘₐₓ for p300 minus Dₘₐₓ for CBP (ΔDₘₐₓ) of at least 30, at least 35, at least 40, or at least 45 percentage points. In certain preferred embodiments, a compound disclosed herein shows selectivity when a compound has a DC₅₀ for p300 at least 30-fold lower than its DC₅₀ for CBP and/or a value of Dₘₐₓ for p300 minus Dₘₐₓ for CBP (ΔDₘₐₓ) of at least 50, at least 55, at least 60, or at least 65 percentage points. In certain more preferred embodiments, a compound disclosed herein shows selectivity when a compound has a DC₅₀ for p300 at least 100-fold lower than its DC₅₀ for CBP and/or a value of Dₘₐₓ for p300 minus Dₘₐₓ for CBP (ΔDₘₐₓ) of at least 70, at least 75, at least 80, at least 85, or at least 90 percentage points. These beneficial properties of the compounds of the present disclosure can be measured according to methods commonly available in the art, such as methods exemplified herein.

Due to the existence of double bonds, the compounds of the present disclosure may be in cis or trans, or Z or E, configuration. It is understood that although one configuration may be depicted in the structure of the compounds or formulae of the present disclosure, the present disclosure also encompasses the other configuration. For example, the compounds or formulae of the present disclosure may be depicted in cis or trans, or Z or E, configuration.

In certain embodiments, a compound of the present disclosure (e.g., a compound of any of the formulae or any individual compounds disclosed herein) is a pharmaceutically acceptable salt. In certain embodiments, a compound of the present disclosure (e.g., a compound of any of the formulae or any individual compounds disclosed herein) is a solvate. In certain embodiments, a compound of the present disclosure (e.g., a compound of any of the formulae or any individual compounds disclosed herein) is a hydrate.

### Pharmaceutically Acceptable Salts

In certain embodiments, the compounds disclosed herein exist as their pharmaceutically acceptable salts. In certain embodiments, the methods disclosed herein include methods of treating diseases by administering such pharmaceutically acceptable salts. In certain embodiments, the methods disclosed herein include methods of treating diseases by administering such pharmaceutically acceptable salts as pharmaceutical compositions.

In certain embodiments, the compounds described herein possess acidic or basic groups and therefor react with any of a number of inorganic or organic bases, and inorganic and organic acids, to form a pharmaceutically acceptable salt. In certain embodiments, these salts are prepared in situ during the final isolation and purification of the compounds disclosed herein, or by separately reacting a purified compound in its free form with a suitable acid or base, and isolating the salt thus formed.

Examples of pharmaceutically acceptable salts include those salts prepared by reaction of the compounds described herein with a mineral, organic acid, or inorganic base, such salts including acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, 7-hydroxybutyrate, hydrochloride, hydrobromide, hydroiodide, 2-hydroxyethanesulfonate, iodide, isobutyrate, lactate, maleate, malonate, methanesulfonate, mandelate metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylateundeconate, and xylenesulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, and muconic acid.

In certain embodiments, those compounds described herein which comprise a free acid group react with a suitable base, such as the hydroxide, carbonate, bicarbonate, or sulfate of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts and the like. Illustrative examples of bases include sodium hydroxide, potassium hydroxide, choline hydroxide, sodium carbonate, N⁺(C₁-C₄ alkyl)₄, and the like.

Representative organic amines useful for the formation of base addition salts include ethylamine, diethylamine, ethylenediamine, ethanolamine, diethanolamine, piperazine, and the like. It should be understood that the compounds described herein also include the quaternization of any basic nitrogen-containing groups they contain. In certain embodiments, water or oil-soluble or dispersible products are obtained by such quaternization.

### Solvates

“Solvate” refers to forms of the compound that are associated with a solvent or water (also referred to as “hydrate”), usually by a solvolysis reaction. This physical association includes hydrogen bonding. Conventional solvents include water, ethanol, acetic acid and the like. The compounds of the disclosure may be prepared e.g., in crystalline form and may be solvated or hydrated. Suitable solvates include pharmaceutically acceptable solvates, such as hydrates, and further include both stoichiometric solvates and non-stoichiometric solvates. In certain instances, the solvate will be capable of isolation, for example when one or more solvent molecules are incorporated in the crystal lattice of the crystalline solid. “Solvate” encompasses both solution-phase and isolable solvates. Representative solvates include hydrates, ethanolates and methanolates.

Those skilled in the art of organic chemistry will appreciate that many organic compounds can form complexes with solvents in which they are reacted or from which they are precipitated or crystallized. These complexes are known as “solvates.” For example, a complex with water is known as a “hydrate.” Solvates are within the scope of the disclosure.

It will also be appreciated by those skilled in organic chemistry that many organic compounds can exist in more than one crystalline form. For example, crystalline form may vary from solvate to solvate. Thus, all crystalline forms or the pharmaceutically acceptable solvates thereof are contemplated and are within the scope of the present disclosure.

In certain embodiments, the compounds described herein exist as solvates. The present disclosure provides for methods of treating diseases by administering such solvates. The present disclosure further provides for methods of treating diseases by administering such solvates as pharmaceutical compositions.

Solvates contain either stoichiometric or non-stoichiometric amounts of a solvent, such as water, ethanol, and the like. Hydrates are formed when the solvent is water, or alcoholates are formed when the solvent is alcohol. Solvates of the compounds described herein can be conveniently prepared or formed during the processes described herein. In addition, the compounds provided herein can exist in unsolvated as well as solvated forms. In general, the solvated forms are considered equivalent to the unsolvated forms for the purposes of the compounds and methods provided herein.

### Isomers (Stereoisomers, Geometric Isomer, Tautomer, Etc.)

It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers.”

Stereoisomers that are not mirror images of one another are termed “diastereomers” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+)- or (−)- isomers respectively). A chiral compound can exist as either individual enantiomer or as a mixture thereof. A mixture containing equal proportions of the enantiomers is termed a “racemic mixture.”

As used herein a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 95% by weight, more than 96% by weight, more than 97% by weight, more than 98% by weight, more than 98.5% by weight, more than 99% by weight, more than 99.2% by weight, more than 99.5% by weight, more than 99.6% by weight, more than 99.7% by weight, more than 99.8% by weight or more than 99.9% by weight, of the enantiomer. In certain embodiments, the weights are based upon total weight of all enantiomers or stereoisomers of the compound.

As used herein and unless otherwise indicated, the term “enantiomerically pure (R)-compound” refers to at least about 95% by weight (R)-compound and at most about 5% by weight (S)-compound, at least about 99% by weight (R)-compound and at most about 1% by weight (S)-compound, or at least about 99.9% by weight (R)-compound and at most about 0.1% by weight (S)-compound. In certain embodiments, the weights are based upon total weight of compound.

As used herein and unless otherwise indicated, the term “enantiomerically pure (S)-compound” refers to at least about 95% by weight (S)-compound and at most about 5% by weight (R)-compound, at least about 99% by weight (S)-compound and at most about 1% by weight (R)-compound or at least about 99.9% by weight (S)-compound and at most about 0.1% by weight (R)-compound. In certain embodiments, the weights are based upon total weight of compound.

In the compositions provided herein, an enantiomerically pure compound or a pharmaceutically acceptable salt, solvate, hydrate or prodrug thereof can be present with other active or inactive ingredients. For example, a pharmaceutical composition comprising enantiomerically pure (R)-compound can comprise, for example, about 90% excipient and about 10% enantiomerically pure (R)-compound. In certain embodiments, the enantiomerically pure (R)-compound in such compositions can, for example, comprise, at least about 95% by weight (R)-compound and at most about 5% by weight (S)-compound, by total weight of the compound. For example, a pharmaceutical composition comprising enantiomerically pure (S)-compound can comprise, for example, about 90% excipient and about 10% enantiomerically pure (S)-compound. In certain embodiments, the enantiomerically pure (S)-compound in such compositions can, for example, comprise, at least about 95% by weight (S)-compound and at most about 5% by weight (R)-compound, by total weight of the compound. In certain embodiments, the active ingredient can be formulated with little or no excipient or carrier.

Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art.

In certain embodiments, the compounds described herein exist as geometric isomers. In certain embodiments, the compounds described herein possess one or more double bonds. The compounds disclosed herein include all cis, trans, syn, anti, entgegen (E), and zusammen (Z) isomers as well as the corresponding mixtures thereof. All geometric forms of the compounds disclosed herein are contemplated and are within the scope of the disclosure.

In certain embodiments, the compounds disclosed herein possess one or more chiral centers and each center exists in the R configuration or S configuration. The compounds disclosed herein include all diastereomeric, enantiomeric, and epimeric forms as well as the corresponding mixtures thereof. All diastereomeric, enantiomeric, and epimeric forms of the compounds disclosed herein are contemplated and are within the scope of the disclosure.

In additional embodiments of the compounds and methods provided herein, mixtures of enantiomers and/or diastereoisomers, resulting from a single preparative step, combination, or interconversion are useful for the applications described herein. In certain embodiments, the compounds described herein are prepared as their individual stereoisomers by reacting a racemic mixture of the compound with an optically active resolving agent to form a pair of diastereoisomeric compounds, separating the diastereomers, and recovering the optically pure enantiomers. In certain embodiments, dissociable complexes are preferred. In certain embodiments, the diastereomers have distinct physical properties (e.g., melting points, boiling points, solubilities, reactivity, etc.) and are separated by taking advantage of these dissimilarities. In certain embodiments, the diastereomers are separated by chiral chromatography, or preferably, by separation/resolution techniques based upon differences in solubility. In certain embodiments, the optically pure enantiomer is then recovered, along with the resolving agent.

### Tautomers

In certain embodiments, compounds described herein exist as tautomers. The compounds described herein include all possible tautomers within the formulas described herein.

Tautomers are compounds that are interconvertible by migration of a hydrogen atom, accompanied by a switch of a single bond and an adjacent double bond. In bonding arrangements where tautomerization is possible, a chemical equilibrium of the tautomers will exist. For example, enols and ketones are tautomers because they are rapidly interconverted by treatment with either acid or base. Another example of tautomerism is the aci- and nitro-forms of phenylnitromethane, that are likewise formed by treatment with acid or base. Tautomeric forms may be relevant to the attainment of the optimal chemical reactivity and biological activity of a compound of interest. All tautomeric forms of the compounds disclosed herein are contemplated and are within the scope of the disclosure. The exact ratio of the tautomers depends on several factors, including temperature, solvent, and pH.

### Conjugates of the Present Disclosure

In certain embodiments, the compound described herein can be directly or indirectly attached to an antibody to form a conjugate.

In certain embodiments, the conjugate comprises a compound described herein directly attached (e.g., via a bond) to an antibody.

In certain embodiments, the conjugate comprises a compound described herein indirectly attached (e.g., via a linker moiety) to an antibody.

In certain embodiments, the conjugate comprises one or more compounds described herein (e.g., prior to conjugation).

Without wishing to be bound by theory, the conjugate may exhibit enhanced delivery properties (e.g., more targeted delivery) compared to an unconjugated compound.

In certain embodiments, the compound described herein is attached to the rest of the conjugate at C of Formula (A) (e.g., wherein a hydrogen of C is replaced by a bond to the rest of the conjugate).

In certain embodiments, the compound described herein is attached to the rest of the conjugate via the linker moiety of Formula (A):

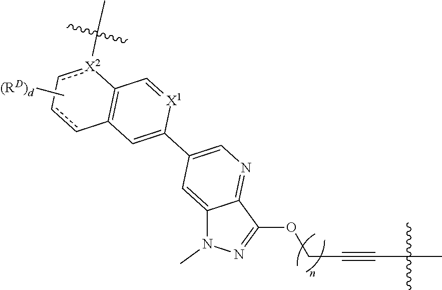

(e.g., wherein a hydrogen of the linker moiety of Formula (A) is replaced by a bond to the rest of the conjugate).

In some embodiments, the conjugate is of Formula (B):

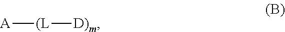

wherein:
A is an antibody;
L is a bond or a linker moiety;
D is a degrader moiety, wherein D, prior to conjugation, is a compound of Formula (A); and
m is an integer selected from about 1 to about 20.

In some embodiments, L is a bond.

In some embodiments, L is a linker moiety.

In some embodiments, L comprises a cleavable bond, wherein cleavage of the bond releases D as a compound of Formula (A) or derivative thereof.

In some embodiments, the antibody, linker moiety (e.g., L), and/or attachment position of a cereblon ligand (e.g., C) to the rest of the conjugate is as described in PCT Application No. PCT/US2023/085698; Hong, B. K. et al. J. Med. Chem. 2023, 66 (1), 140-148; Dragovich, P. S. Chem. Soc. Rev. 2022, 51(10), 3886-3897; and Poudel, Y. B. et al. J. Med. Chem. 2024, 67 (18), 15996-16001 (each incorporated herein by reference).

In some embodiments, m is about 1, about 5, about 10, about 15, or about 20. In some embodiments, m is an integer selected from about 1 to about 15, about 1 to about 10, about 1 to about 5, about 5 to about 20, about 10 to about 20, about 15 to about 20, or about 5 to about 15.

### Pharmaceutical Compositions

In certain embodiments, the compound or conjugate described herein is administered as a pure chemical. In certain embodiments, the compound or conjugate described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21^{st} Ed. Mack Pub. Co., Easton, PA (2005)).

Accordingly, the present disclosure provides pharmaceutical compositions comprising a compound or conjugate described herein, or a pharmaceutically acceptable salt, solvate, or stereoisomer thereof, and one or more pharmaceutically acceptable excipient.

In certain embodiments, the compound or conjugate provided herein is substantially pure, in that it contains less than about 5%, less than about 1%, or less than about 0.1% of other organic small molecules, such as unreacted intermediates or synthesis by-products that are created, for example, in one or more of the steps of a synthesis method.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses are generally determined using experimental models and/or clinical trials. The optimal dose depends upon the body mass, weight, or blood volume of the patient.

In certain embodiments, the pharmaceutical composition is formulated for oral, topical (including buccal and sublingual), rectal, vaginal, transdermal, parenteral, intrapulmonary, intradermal, intrathecal and epidural and intranasal administration. Parenteral administration includes intramuscular, intravenous, intraarterial, intraperitoneal, or subcutaneous administration. In certain embodiments, the pharmaceutical composition is formulated for intravenous injection, oral administration, inhalation, nasal administration, topical administration, or ophthalmic administration. In certain embodiments, the pharmaceutical composition is formulated for oral administration. In certain embodiments, the pharmaceutical composition is formulated for intravenous injection. In certain embodiments, the pharmaceutical composition is formulated as a tablet, a pill, a capsule, a liquid, an inhalant, a nasal spray solution, a suppository, a suspension, a gel, a colloid, a dispersion, a suspension, a solution, an emulsion, an ointment, a lotion, an eye drop, or an ear drop. In certain embodiments, the pharmaceutical composition is formulated as a tablet.

### Preparation and Characterization of the Compounds

The compounds of the present disclosure can be prepared in a number of ways well known to those skilled in the art of organic synthesis. By way of example, the compounds of the present disclosure can be synthesized using the methods described below, together with synthetic methods known in the art of synthetic organic chemistry, or variations thereon as appreciated by those skilled in the art. The compounds of the present disclosure (i.e., a compound of the present application (e.g., a compound of any of the formulae or any individual compounds disclosed herein)) can be synthesized by following the general synthetic scheme below as well as the steps outlined in the examples, schemes, procedures, and/or synthesis described herein (e.g., Examples).

### General Synthetic Scheme

A compound of Formula I may be prepared according to the procedures shown in SCHEME 1.

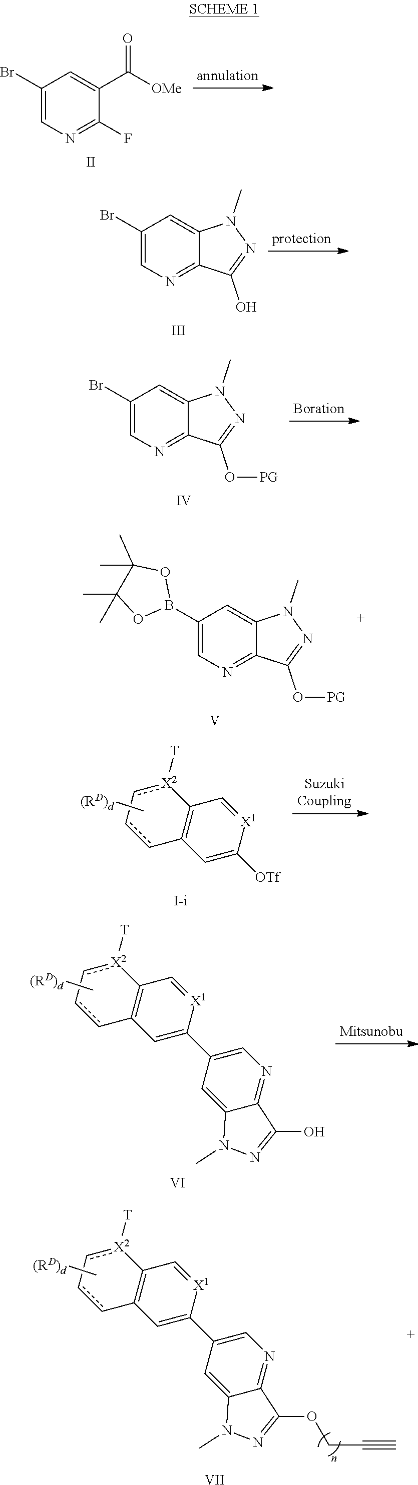

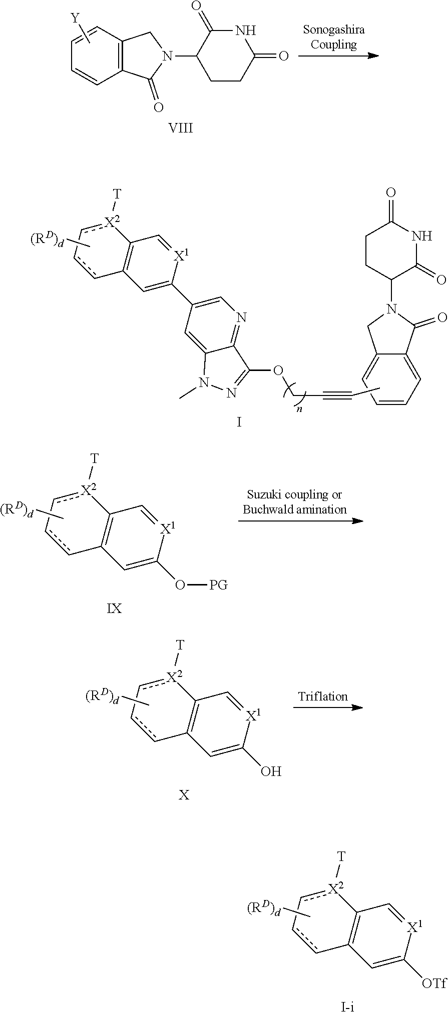

According to SCHEME 1, commercially available compound of Formula II is reacted with methyl hydrazine; in a suitable solvent such as dioxane, EtOH or i-PrOH and the like; at temperatures ranging from 50° C. to 80° C.; to provide annulated compound of Formula III. An aza indazole compound of Formula III is protected with a suitable protecting reagent such as TBSCl and the like; in a base such as TEA, imidazole, DIPEA, and the like; at temperatures ranging from 20° C. to 60° C., preferably 40° C.; in a suitable solvent such as dioxane, THF or DMF and the like to provide a compound of Formula IV. A bromo compound of Formula IV is reacted with a commercially available 4,4,4′,4′,5,5,5′,5′-octamethyl-2,2′-bi(1,3,2-dioxaborolane); with a suitable catalyst such as PdCl₂(dppf), Pd(Ph₃P)₄ and the like; with a suitable base such as potassium acetate, sodium carbonate, and the like; in a suitable solvent such as 1,4-dioxane, DMSO and the like; at temperatures ranging from 60° C. to 120° C.; to provide boronic ester compound of Formula V. A boronic ester compound of Formula V is reacted under Suzuki coupling condition; with a synthetically known Intermediate I-i; with a suitable catalyst such as Pd(Ph₃P)₄, Pd₂(dba)₃ and the like; with a suitable base such as potassium phosphate, sodium carbonate, and the like; in a suitable solvent such as 1,4-dioxane, water and the like; at temperatures ranging from 80° C. to 120° C.; to provide coupled compound of Formula VI. A coupled aza indazole compound of Formula VI is reacted under Mitsunobu condition; with suitable alkynyl alcohol such as prop-2-yn-1-ol, but-3-yn-1-ol, pent-4-yn-1-ol and the like; in a suitable solvent such as THF, DCM and the like; with a suitable coupling agent such as DEAD, DIAD and the like; with a suitable co-agent such as Ph₃P, Bu₃P and the like; at temperatures ranging from 0° C. to 60° C.; to provide the coupled compound of Formula VII. A coupled compound of Formula VII is reacted with commercially available or synthetically accessible substituted iso-indolinone compound of Formula VIII (Y=halogen); with a suitable catalyst such as PdCl₂(Ph₃P)₂, PdCl₂(dppf) and the like; with a suitable co-catalyst such as CuI, CuBr, and the like; with a suitable base such as TEA, DIPEA, and the like; in a suitable solvent such as TEA, THF and the like; at temperatures ranging from 25° C. to 80° C.; to provide coupled compound of Formula I.

A halogenated compound of Formula IX (when X²═CY, Y=halogen) is reacted under Suzuki coupling condition; with a synthetically known intermediate boronic acid or ester T-B(OH)₂; with a suitable catalyst such as Pd(Ph₃P)₄, Pd₂(dba)₃ and the like; with a suitable base such as potassium phosphate, sodium carbonate, and the like; in a suitable solvent such as 1,4-dioxane, water and the like; at temperatures ranging from 80° C. to 120° C.; to provide coupled compound of Formula X. Or an amino compound of Formula IX (when X²═NH) is reacted under Buchwald amination condition; with a synthetically known intermediate halide or triflate T-Y (Y=halide or OTf); with a suitable catalyst such as Pd(OAc)₂, Pd₂(dba)₃ and the like; with s suitable ligand such as xantphos, Xphos and the like; with a suitable base such as sodium t-butoxide, cesium carbonate, and the like; in a suitable solvent such as 1,4-dioxane, water and the like; at temperatures ranging from 80° C. to 120° C.; to provide coupled compound of Formula X. A compound of Formula X is protected with a suitable protecting reagent such as Tf₂O and the like; in a base such as TEA, imidazole, DIPEA, and the like; at temperatures ranging from −10° C. to 20° C., preferably 0° C.; in a suitable solvent such as DCM, THF or DMF and the like to provide a compound of Formula I-i.

Those skilled in the art will recognize if a stereocenter exists in the compounds of the present disclosure (e.g., a compound of any of the formulae or any individual compounds disclosed herein). Accordingly, the present disclosure includes both possible stereoisomers (unless specified in the synthesis) and includes not only racemic compound but the individual enantiomers and/or diastereomers as well. When a compound is desired as a single enantiomer or diastereomer, it may be obtained by stereospecific synthesis or by resolution of the final product or any convenient intermediate. Resolution of the final product, an intermediate, or a starting material may be affected by any suitable method known in the art. See, for example, “Stereochemistry of Organic Compounds” by E. L. Eliel, S. H. Wilen, and L. N. Mander (Wiley-Interscience, 1994).

The compounds used in the reactions described herein are made according to organic synthesis techniques known to those skilled in this art, starting from commercially available chemicals and/or from compounds described in the chemical literature. “Commercially available chemicals” are obtained from standard commercial sources including Acros Organics (Pittsburgh, PA), Aldrich Chemical (Milwaukee, WI, including Sigma Chemical and Fluka) (Pittsburgh, PA).

Suitable reference books and treatises that detail the synthesis of reactants useful in the preparation of compounds described herein, or provide references to articles that describe the preparation, include for example, “Synthetic Organic Chemistry,” John Wiley & Sons, Inc., New York; S. R. Sandler et al., “Organic Functional Group Preparations,” 2nd Ed., Academic Press, New York, 1983; H. O. House, “Advanced Organic Chemistry: Reactions, Mechanisms and Structure,” 4th Ed., Wiley-Interscience, New York, 1992; and “Chemistry of Functional Groups” John Wiley & Sons, in 73 volumes.

Specific and analogous reactants are optionally identified through the indices of known chemicals prepared by the Chemical Abstract Service of the American Chemical Society, which are available in most public and university libraries, as well as through on-line. Chemicals that are known but not commercially available in catalogs are optionally prepared by custom chemical synthesis houses, where many of the standard chemical supply houses (e.g., those listed above) provide custom synthesis services. A reference for the preparation and selection of pharmaceutical salts of the compounds described herein is P. H. Stahl & C. G. Wermuth “Handbook of Pharmaceutical Salts,” Verlag Helvetica Chimica Acta, Zurich, 2002.

### Biological Assays

The biological activities of the compounds of the present disclosure can be assessed with methods and assays known in the art.

The binding potencies of the compounds to p300 may be determined using HTRF assay technology. HTRF signals may be measured by displacing the fluorescent probe, 5-(8-(7-acetyl-3-(tetrahydro-2H-pyran-4-yl)-5,6,7,8-tetrahydroimidazo[1,5-a]pyrazin-1-yl)isoquinolin-3-yl)-N-(2-(2-(2-(3-(3′,6′-dihydroxy-3-oxo-3H-spiro[isobenzofuran-1,9′-xanthen]-5-yl)thioureido)ethoxy)ethoxy)ethyl)picolinamide, from FLAG-tagged p300 with the tested compounds. Data analysis may be performed using TIBCO Spotfire using a four-parameter dose response curve to determine the IC₅₀ of the tested compounds.

The binding potencies of the compounds to CBP may be determined using HTRF assay technology. HTRF signals may be measured by displacing the fluorescent probe, 5-(8-(7-acetyl-3-(tetrahydro-2H-pyran-4-yl)-5,6,7,8-tetrahydroimidazo[1,5-a]pyrazin-1-yl)isoquinolin-3-yl)-N-(2-(2-(2-(3-(3′,6′-dihydroxy-3-oxo-3H-spiro[isobenzofuran-1,9′-xanthen]-5-yl)thioureido)ethoxy)ethoxy)ethyl)picolinamide, from a FLAG-tagged (C-terminal) and His-tagged (N-terminal) CBP construct with the tested compounds. Data analysis may be performed using TIBCO Spotfire using a four-parameter dose response curve to determine the IC₅₀ of the tested compounds.

The cellular degradation activities of the compounds against p300 may be measured by in-cell Western blot technology in H1299 cells with the tested compounds at initial concentrations of 0.0005, 0.0015, 0.0046, 0.014, 0.041, 0.12, 0.37, 1.1, 3.3, and 10 μM for 16 hours. The resulting protein concentration may be assessed using an anti-p300 primary antibody (anti-human p300 clone D8Z4E) and an IRDye 800CW secondary antibody (IRDye 800CW goat anti-rabbit IgG). The resultant fluorescence may be measured with the LI-COR Odyssey CLx instrument. Dose-response curves may be generated and analyzed using TIBCO Spotfire to determine the DC₅₀ and Dₘₐₓ of the tested compounds.

The cellular degradation activities of the compounds against CBP may be measured by in-cell Western blot technology in H1299 cells with the tested compounds at initial concentrations of 0.0005, 0.0015, 0.0046, 0.014, 0.041, 0.12, 0.37, 1.1, 3.3, and 10 μM for 16 hours. The resulting protein concentration may be assessed using an anti-CBP primary antibody (anti-human CBP clone D6C5) and an IRDye 800CW secondary antibody (IRDye 800CW goat anti-rabbit IgG). The resultant fluorescence may be measured with the LI-COR Odyssey CLx instrument. Dose-response curves may be generated and analyzed using TIBCO Spotfire to determine the DC₅₀ and Dₘₐₓ of the tested compounds.

The ability of the compounds to inhibit cellular proliferation may be assessed by Cell Titer-Glo technology in H1299 wild-type, H1299 p300 knock-out, and H1299 CBP knock-out cell lines. The tested compounds may be assessed at initial concentrations of 0.0005, 0.0015, 0.0046, 0.014, 0.041, 0.12, 0.37, 1.1, 3.3, and 10 μM for 6 days. Cell growth may be assessed using the Cell Titer-Glo Luminescent Cell Viability reagent and a Perkin Elmer Envision instrument. The cell growth may be normalized to DMSO and compared to day 0 control measurements. Dose-response curves may be generated and analyzed using TIBCO Spotfire to determine the gIC₅₀ and the growth-death index (i.e. the percentage of remaining cell growth or cell death observed relative to untreated cells).

### Methods of Use

In certain aspects, the present disclosure provides methods of degrading a protein in a subject or biological sample comprising administering the compound disclosed herein to the subject or contacting the biological sample with the compound disclosed herein.

In certain aspects, the present disclosure provides uses of the compound disclosed herein in the manufacture of a medicament for degrading a protein in a subject or biological sample.

In certain aspects, the present disclosure provides compounds disclosed herein for use in degrading a protein in a subject or biological sample.

In certain aspects, the present disclosure provides methods of degrading a protein in a subject or biological sample comprising administering the conjugate disclosed herein to the subject or contacting the biological sample with the compound disclosed herein.

In certain aspects, the present disclosure provides uses of the conjugate disclosed herein in the manufacture of a medicament for degrading a protein in a subject or biological sample.

In certain aspects, the present disclosure provides conjugates disclosed herein for use in degrading a protein in a subject or biological sample.

In certain embodiments, the protein is p300. In certain embodiments, the protein is CBP.

In certain aspects, the present disclosure provides methods for treating a disease or disorder.

In certain aspects, the present disclosure provides uses of the compounds disclosed herein in the manufacture of a medicament for treating a disease or disorder.

In certain aspects, the present disclosure provides compounds disclosed herein for treating a disease or disorder.

In certain aspects, the present disclosure provides uses of the conjugates disclosed herein in the manufacture of a medicament for treating a disease or disorder.

In certain aspects, the present disclosure provides conjugates disclosed herein for treating a disease or disorder.

In certain embodiments, the disease or disorder is a p300-mediated disease or disorder.

In certain embodiments, the disease or disorder is a CBP-mediated disease or disorder.

In certain embodiments, the disease or disorder is driven by gene activation such as cancer, inflammatory disorders, or autoimmune diseases.

In certain embodiments, the disease or disorder is selected from the group consisting of acoustic neuroma, acute leukemia, acute lymphocytic leukemia, acute myelocytic leukemia, acute T-cell leukemia, basal cell carcinoma, bile duct carcinoma, bladder cancer, brain choriocarcinoma, chronic leukemia, chronic lymphocytic leukemia, chronic myelocytic leukemia, chronic myelogenous leukemia, colon cancer, colorectal cancer, craniopharyngioma, cystadenocarcinoma, diffuse large B-cell lymphoma, dysproliferative changes, embryonal carcinoma, endometrial cancer, endotheliosarcoma, ependymoma, epithelial carcinoma, erythroleukemia, esophageal cancer, estrogen alfa receptor positive breast cancer, essential thrombocythemia, Ewing's tumor, fibrosarcoma, follicular lymphoma, germ cell testicular cancer, glioma, glioblastoma, gliosarcoma, heavy chain disease, head and neck cancer, hemangioblastoma, hepatoma, hepatocellular cancer, hormone insensitive prostate cancer, leiomyosarcoma, leukemia, liposarcoma, lung cancer, lymphagioendotheliosarcoma, lymphangiosarcoma, lymphoblastic leukemia, lymphoma, lymphoid malignancies of T- cell or B-cell origin, medullary carcinoma, medulloblastoma, melanoma, meningioma, mesothelioma, multiple myeloma, myelogenous leukemia, myeloma, myxosarcoma, neuroblastoma, NUT midline carcinoma (NMC), non-small cell lung cancer, oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumors, uterine cancer, and Wilms' tumor.

In certain embodiments, the disease or disorder is selected from the group consisting of Addison's disease, acute gout, ankylosing spondylitis, asthma, atherosclerosis, Behcet's disease, bullous skin diseases, chronic obstructive pulmonary disease, Crohn's disease, dermatitis, eczema, giant cell arteritis, fibrosis, glomerulonephritis, hepatic vascular occlusion, hepatitis, hypophysitis, immunodeficiency syndrome, inflammatory bowel disease, Kawasaki disease, lupus nephritis, multiple sclerosis, myocarditis, myositis, nephritis, organ transplant rejection, osteoarthritis, pancreatitis, pericarditis, Polyarteritis nodosa, pneumonitis, primary biliary cirrhosis, psoriasis, psoriatic arthritis, rheumatoid arthritis, scleritis, sclerosing cholangitis, sepsis, systemic lupus erythematosus, Takayasu's Arteritis, toxic shock, thyroiditis, type I diabetes, ulcerative colitis, uveitis, vitiligo, vasculitis, and Wegener's granulomatosis.

In certain embodiments, the disease or disorder is selected from the group consisting of prostate cancer, lung cancer, breast cancer, pancreatic cancer, colorectal cancer, and melanoma.

In certain embodiments, the subject is a mammal.

In certain embodiments, the subject is a human.

### Definitions

As used in the specification and appended claims, unless specified to the contrary, the following terms have the meaning indicated below.

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley &amp; Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPFC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. F. Eliel, Ed., Univ. of Notre Dame Press, Notre Dame, IN 1972).

The invention additionally encompasses compounds described herein as individual isomers substantially free of other isomers, and alternatively, as mixtures of various isomers.

When a range of values is listed, it is intended to encompass each value and sub-range within the range. For example, “C₁-C₆ alkyl” is intended to encompass, C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl.

The following terms are intended to have the meanings presented therewith below and are useful in understanding the description and intended scope of the present invention. When describing the invention, which may include compounds, pharmaceutical compositions containing such compounds and methods of using such compounds and compositions, the following terms, if present, have the following meanings unless otherwise indicated. It should also be understood that when described herein any of the moieties defined forth below may be substituted with a variety of substituents, and that the respective definitions are intended to include such substituted moieties within their scope as set out below. Unless otherwise stated, the term “substituted” is to be defined as set out below. It should be further understood that the terms “groups” and “radicals” can be considered interchangeable when used herein. The articles “a” and “an” may be used herein to refer to one or to more than one (i.e., at least one) of the grammatical objects of the article. By way of example “an analogue” means one analogue or more than one analogue.

“Alkyl” as used herein, refers to a radical of a straight-chain or branched saturated hydrocarbon group having from 1 to 20 carbon atoms (“C₁-C₂ alkyl”). In certain embodiments, an alkyl group has 1 to 12 carbon atoms (“C₁-C₁₂ alkyl”). In certain embodiments, an alkyl group has 1 to 10 carbon atoms (“C₁-C₁₀alkyl”). In certain embodiments, an alkyl group has 1 to 9 carbon atoms (“C₁-C₉ alkyl”). In certain embodiments, an alkyl group has 1 to 8 carbon atoms (“C₁-C₈ alkyl”). In certain embodiments, an alkyl group has 1 to 7 carbon atoms (“C₁-C₇ alkyl”). In certain embodiments, an alkyl group has 1 to 6 carbon atoms (“C₁-C₆ alkyl,” which is also referred to herein as “lower alkyl”). In certain embodiments, an alkyl group has 1 to 5 carbon atoms (“C₁-C₅ alkyl”). In certain embodiments, an alkyl group has 1 to 4 carbon atoms (“C₁-C₄ alkyl”). In certain embodiments, an alkyl group has 1 to 3 carbon atoms (“C₁-C₃ alkyl”). In certain embodiments, an alkyl group has 1 to 2 carbon atoms (“C₁-C₂ alkyl”). In certain embodiments, an alkyl group has 1 carbon atom (“C₁ alkyl”). Examples of C₁-C₆ alkyl groups include methyl (C₁), ethyl (C₂), n-propyl (C₃), isopropyl (C₃), n-butyl (C₄), tert-butyl (C₄), sec-butyl (C₄), isobutyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Unless otherwise specified, each instance of an alkyl group is independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent. In certain embodiments, the alkyl group is unsubstituted C₁-C₁₀ alkyl (e.g., —CH₃). In certain embodiments, the alkyl group is substituted C₁-C₁₀ alkyl. Common alkyl abbreviations include Me (—CH₃), Et (—CH₂CH₃), i-Pr (—CH(CH₃)₂), n-Pr (—CH₂CH₂CH₃), n-Bu (—CH₂CH₂CH₂CH₃), or i-Bu (—CH₂CH(CH₃)₂).

“Alkenyl” as used herein, refers to a radical of a straight-chain or branched hydrocarbon group having from 2 to 20 carbon atoms, one or more carbon-carbon double bonds (e.g., 1, 2, 3, or 4 carbon-carbon double bonds), and optionally one or more carbon-carbon triple bonds (e.g., 1, 2, 3, or 4 carbon-carbon triple bonds) (“C₂-C₂₀ alkenyl”). In certain embodiments, alkenyl does not contain any triple bonds. In certain embodiments, an alkenyl group has 2 to 10 carbon atoms (“C₂-C₁₀ alkenyl”). In certain embodiments, an alkenyl group has 2 to 9 carbon atoms (“C₂-C₉ alkenyl”). In certain embodiments, an alkenyl group has 2 to 8 carbon atoms (“C₂-C₈ alkenyl”). In certain embodiments, an alkenyl group has 2 to 7 carbon atoms (“C₂-C₇ alkenyl”). In certain embodiments, an alkenyl group has 2 to 6 carbon atoms (“C₂-C₆ alkenyl”). In certain embodiments, an alkenyl group has 2 to 5 carbon atoms (“C₂-C₅ alkenyl”). In certain embodiments, an alkenyl group has 2 to 4 carbon atoms (“C₂-C₄ alkenyl”). In certain embodiments, an alkenyl group has 2 to 3 carbon atoms (“C₂-C₃ alkenyl”). In certain embodiments, an alkenyl group has 2 carbon atoms (“C₂ alkenyl”). The one or more carbon-carbon double bonds can be internal (such as in 2-butenyl) or terminal (such as in 1-butenyl). Examples of C₂-C₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂-C₆ alkenyl groups include the aforementioned C₂-C₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent. In certain embodiments, the alkenyl group is unsubstituted C₂-C₁₀ alkenyl. In certain embodiments, the alkenyl group is substituted C₂-C₁₀ alkenyl.

“Alkynyl” as used herein, refers to a radical of a straight-chain or branched hydrocarbon group having from 2 to 20 carbon atoms, one or more carbon-carbon triple bonds (e.g., 1, 2, 3, or 4 carbon-carbon triple bonds), and optionally one or more carbon-carbon double bonds (e.g., 1, 2, 3, or 4 carbon-carbon double bonds) (“C₂-C₂₀ alkynyl”). In certain embodiments, alkynyl does not contain any double bonds. In certain embodiments, an alkynyl group has 2 to 10 carbon atoms (“C₂-C₁₀ alkynyl”). In certain embodiments, an alkynyl group has 2 to 9 carbon atoms (“C₂-C₉ alkynyl”). In certain embodiments, an alkynyl group has 2 to 8 carbon atoms (“C₂-C₈ alkynyl”). In certain embodiments, an alkynyl group has 2 to 7 carbon atoms (“C₂-C₇ alkynyl”). In certain embodiments, an alkynyl group has 2 to 6 carbon atoms (“C₂-C₆ alkynyl”). In certain embodiments, an alkynyl group has 2 to 5 carbon atoms (“C₂-C₅ alkynyl”). In certain embodiments, an alkynyl group has 2 to 4 carbon atoms (“C₂-C₄ alkynyl”). In certain embodiments, an alkynyl group has 2 to 3 carbon atoms (“C₂-C₃ alkynyl”). In certain embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂-C₄ alkynyl groups include, without limitation, ethynyl (C₂), 1-propynyl (C₃), 2-propynyl (C₃), 1-butynyl (C₄), 2-butynyl (C₄), and the like. Examples of C₂-C₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently optionally substituted, i.e., unsubstituted (an “unsubstituted alkynyl”) or substituted (a “substituted alkynyl”) with one or more substituents; e.g., for instance from 1 to 5 substituents, 1 to 3 substituents, or 1 substituent. In certain embodiments, the alkynyl group is unsubstituted C₂-C₁₀ alkynyl. In certain embodiments, the alkynyl group is substituted C₂-C₁₀ alkynyl.

“Aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆-C₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl).

Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, and trinaphthalene. Particular aryl groups include phenyl, naphthyl, indenyl, and tetrahydronaphthyl. Unless otherwise specified, each instance of an aryl group is independently optionally substituted, i.e., unsubstituted (an “unsubstituted aryl”) or substituted (a “substituted aryl”) with one or more substituents. In certain embodiments, the aryl group is unsubstituted C₆-C₁₄ aryl. In certain embodiments, the aryl group is substituted C₆-C₁₄ aryl.

“Heteroaryl” refers to a radical of a 5- to 14-membered monocyclic or polycyclic 4n+2 aromatic ring system (e.g., having 6, 10, or 14 π electrons shared in a cyclic array) having ring carbon atoms and 1-8 ring heteroatoms provided in the aromatic ring system, wherein each heteroatom is independently selected from nitrogen, oxygen and sulfur (“5- to 14-membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings.

“Heteroaryl” also includes ring systems wherein the heteroaryl group, as defined above, is fused with one or more aryl groups wherein the point of attachment is either on the heteroaryl or the one or more aryl groups, and in such instances, the number of ring members designates the total number of ring members in the fused (aryl/heteroaryl) ring system. When substitution is indicated in such instances, unless otherwise specified, substitution can occur on either the heteroaryl or the one or more aryl groups. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In certain embodiments, a heteroaryl is a 5- to 10-membered aromatic ring system having ring carbon atoms and 1-4 ring heteroatoms provided in the aromatic ring system, wherein each heteroatom is independently selected from nitrogen, oxygen, and sulfur (“5- to 10-membered heteroaryl”). In certain embodiments, a heteroaryl is a 5- to 9-membered aromatic ring system having ring carbon atoms and 1-4 ring heteroatoms provided in the aromatic ring system, wherein each heteroatom is independently selected from nitrogen, oxygen, and sulfur (“5- to 9-membered heteroaryl”). In certain embodiments, a heteroaryl is a 5- to 8-membered aromatic ring system having ring carbon atoms and 1-4 ring heteroatoms provided in the aromatic ring system, wherein each heteroatom is independently selected from nitrogen, oxygen, and sulfur (“5- to 8-membered heteroaryl”). In certain embodiments, a heteroaryl group is a 5- to 6-membered aromatic ring system having ring carbon atoms and 1-4 ring heteroatoms provided in the aromatic ring system, wherein each heteroatom is independently selected from nitrogen, oxygen, and sulfur (“5- to 6-membered heteroaryl”). In certain embodiments, the 5- to 6-membered heteroaryl has 1-3 ring heteroatoms independently selected from nitrogen, oxygen, and sulfur. In certain embodiments, the 5- to 6-membered heteroaryl has 1-2 ring heteroatoms independently selected from nitrogen, oxygen, and sulfur. In certain embodiments, the 5- to 6-membered heteroaryl has 1 ring heteroatom selected from nitrogen, oxygen, and sulfur. Unless otherwise specified, each instance of a heteroaryl group is independently optionally substituted, i.e., unsubstituted (an “unsubstituted heteroaryl”) or substituted (a “substituted heteroaryl”) with one or more substituents. In certain embodiments, the heteroaryl group is unsubstituted 5- to 14-membered heteroaryl. In certain embodiments, the heteroaryl group is substituted 5- to 14-membered heteroaryl.

Exemplary 5-membered heteroaryl containing one heteroatom include, without limitation, pyrrolyl, furanyl and thiophenyl. Exemplary 5-membered heteroaryl containing two heteroatoms include, without limitation, imidazolyl, pyrazolyl, oxazolyl, isoxazolyl, thiazolyl, and isothiazolyl. Exemplary 5-membered heteroaryl containing three heteroatoms include, without limitation, triazolyl, oxadiazolyl, and thiadiazolyl. Exemplary 5-membered heteroaryl containing four heteroatoms include, without limitation, tetrazolyl. Exemplary 6-membered heteroaryl containing one heteroatom include, without limitation, pyridinyl. Exemplary 6-membered heteroaryl containing two heteroatoms include, without limitation, pyridazinyl, pyrimidinyl, and pyrazinyl. Exemplary 6-membered heteroaryl containing three or four heteroatoms include, without limitation, triazinyl and tetrazinyl, respectively. Exemplary 7-membered heteroaryl containing one heteroatom include, without limitation, azepinyl, oxepinyl, and thiepinyl. Exemplary 5,6-bicyclic heteroaryl include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl include, without limitation, naphthyridinyl, pteridinyl, quinolinyl, isoquinolinyl, cinnolinyl, quinoxalinyl, phthalazinyl, and quinazolinyl.

“Carbocyclyl” or “carbocycle” refers to a radical of a non-aromatic cyclic hydrocarbon group having from 3 to 12 ring carbon atoms (“C₃-C₁₂ carbocyclyl”) and zero heteroatoms in the nonaromatic ring system. In certain embodiments, a carbocyclyl group has 3 to 10 ring carbon atoms (“C₃₋₁₀ carbocyclyl”). In certain embodiments, a carbocyclyl group has 3 to 8 ring carbon atoms (“C₃-C₈ carbocyclyl”). In certain embodiments, a carbocyclyl group has 3 to 6 ring carbon atoms (“C₃-C₆ carbocyclyl”). In certain embodiments, a carbocyclyl group has 5 to 12 ring carbon atoms (“C₅-C₁₂ carbocyclyl”). In certain embodiments, a carbocyclyl group has 5 to 10 ring carbon atoms (“C₅-C₁₀ carbocyclyl”). In certain embodiments, a carbocyclyl group has 5 to 8 ring carbon atoms (“C₅-C₈ carbocyclyl”). In certain embodiments, a carbocyclyl group has 5 or 6 ring carbon atoms (“C₅-C₆ carbocyclyl”). Exemplary C₃-C₆ carbocyclyl include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃-C₈ carbocyclyl include, without limitation, the aforementioned C₃-C₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃-C₁₀ carbocyclyl include, without limitation, the aforementioned C₃-C₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like.

In certain embodiments, “carbocyclyl” is a monocyclic, saturated carbocyclyl group having from 3 to 12 ring carbon atoms (“C₃-C₁₂ carbocyclyl”). In certain embodiments, “carbocyclyl” is a monocyclic, saturated carbocyclyl group having from 3 to 10 ring carbon atoms (“C₃-C₁₀ carbocyclyl”). In certain embodiments, “carbocyclyl” is a monocyclic, saturated carbocyclyl group having from 3 to 8 ring carbon atoms (“C₃-C₈ carbocyclyl”). In certain embodiments, “carbocyclyl” is a monocyclic, saturated carbocyclyl group having from 3 to 6 ring carbon atoms (“C₃-C₆ carbocyclyl”). In certain embodiments, “carbocyclyl” is a monocyclic, saturated carbocyclyl group having from 5 to 12 ring carbon atoms (“C₅-C₁₂ carbocyclyl”). In certain embodiments, a carbocyclyl group has 5 to 10 ring carbon atoms (“C₅-C₁₀ carbocyclyl”). In certain embodiments, a carbocyclyl group has 5 to 8 ring carbon atoms (“C₅-C₈ carbocyclyl”). In certain embodiments, “carbocyclyl” is a monocyclic, saturated carbocyclyl group having 5 or 6 ring carbon atoms (“C₅-C₆ carbocyclyl”). Examples of C₅-C₆ carbocyclyl include cyclopentyl (C₅) and cyclohexyl (C₅). Examples of C₃-C₆ carbocyclyl include the aforementioned C₅-C₆ carbocyclyl groups as well as cyclopropyl (C₃) and cyclobutyl (C₄). Examples of C₃-C₈ carbocyclyl include the aforementioned C₃-C₆ carbocyclyl groups as well as cycloheptyl (C₇) and cyclooctyl (C₈). Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is unsubstituted C₃-C₁₂ carbocyclyl. In certain embodiments, the carbocyclyl group is substituted C₃-C₁₂ carbocyclyl.

As the foregoing examples illustrate, in certain embodiments, the carbocyclyl group is either monocyclic (“monocyclic carbocyclyl”) or polycyclic (“polycyclic carbocyclyl”) that contains a fused, bridged or spiro ring system and can be saturated or can be partially unsaturated. Unless otherwise specified, each instance of a carbocyclyl group is independently optionally substituted, i.e., unsubstituted (an “unsubstituted carbocyclyl”) or substituted (a “substituted carbocyclyl”) with one or more substituents. In certain embodiments, the carbocyclyl group is unsubstituted C₃-C₁₂ carbocyclyl. In certain embodiments, the carbocyclyl group is a substituted C₃-C₁₂ carbocyclyl.

“Fused carbocyclyl” or “fused carbocycle” refers to ring systems wherein the carbocyclyl group, as defined above, is fused with, i.e., share two common atoms (as such, share one common bond), one or more carbocyclyl groups, as defined above, wherein the point of attachment is on any of the fused rings. In such instances, the number of carbons designates the total number of carbons in the fused ring system. When substitution is indicated, unless otherwise specified, substitution can occur on any of the fused rings.

“Spiro carbocyclyl” or “spiro carbocycle” refers to ring systems wherein the carbocyclyl group, as defined above, form spiro structure with, i.e., share one common atom with, one or more carbocyclyl groups, as defined above, wherein the point of attachment is on the carbocyclyl rings in which the spiro structure is embedded. In such instances, the number of carbons designates the total number of carbons of the carbocyclyl rings in which the spiro structure is embedded. When substitution is indicated, unless otherwise specified, substitution can occur on the carbocyclyl rings in which the spiro structure is embedded.

“Bridged carbocyclyl” or “bridged carbocycle” refers to ring systems wherein the carbocyclyl group, as defined above, form bridged structure with, i.e., share more than two atoms (as such, share more than one bonds) with, one or more carbocyclyl groups, as defined above, wherein the point of attachment is on any of the carbocyclyl rings in which the bridged structure is embedded. In such instances, the number of carbons designates the total number of carbons of the carbocyclyl rings in which the bridged structure is embedded. When substitution is indicated, unless otherwise specified, substitution can occur on any of the carbocyclyl rings in which the bridged structure is embedded.

“Heterocyclyl” or “heterocycle” refers to a radical of a 3- to 12-membered non-aromatic ring system having ring carbon atoms and 1 to 4 ring heteroatoms, wherein each heteroatom is independently selected from nitrogen, oxygen, sulfur, boron, phosphorus, and silicon (“3- to 12-membered heterocyclyl”). In heterocyclyl groups that contain one or more nitrogen atoms, the point of attachment can be a carbon or nitrogen atom, as valency permits. Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5 membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

In certain embodiments, a heterocyclyl group is a 5- to 12-membered non-aromatic ring system having ring carbon atoms and 1-4 ring heteroatoms, wherein each heteroatom is independently selected from nitrogen, oxygen, sulfur, boron, phosphorus, and silicon (“5- to 12-membered heterocyclyl”). In certain embodiments, a heterocyclyl group is a 5- to 10-membered non-aromatic ring system having ring carbon atoms and 1-4 ring heteroatoms, wherein each heteroatom is independently selected from nitrogen, oxygen, sulfur, boron, phosphorus, and silicon (“5- to 10-membered heterocyclyl”). In certain embodiments, a heterocyclyl group is a 5- to 8-membered non-aromatic ring system having ring carbon atoms and 1-4 ring heteroatoms, wherein each heteroatom is independently selected from nitrogen, oxygen, and sulfur (“5- to 8-membered heterocyclyl”). In certain embodiments, a heterocyclyl group is a 5- to 6-membered non-aromatic ring system having ring carbon atoms and 1-4 ring heteroatoms, wherein each heteroatom is independently selected from nitrogen, oxygen, and sulfur (“5- to 6-membered heterocyclyl”). In certain embodiments, the 5- to 6-membered heterocyclyl has 1-3 ring heteroatoms selected from nitrogen, oxygen, and sulfur. In certain embodiments, the 5- to 6-membered heterocyclyl has 1-2 ring heteroatoms selected from nitrogen, oxygen, and sulfur. In certain embodiments, the 5- to 6-membered heterocyclyl has one ring heteroatom selected from nitrogen, oxygen, and sulfur.

As the foregoing examples illustrate, in certain embodiments, a heterocyclyl group can either be monocyclic (“monocyclic heterocyclyl”) or polycyclic (“polycyclic heterocyclyl”) that contains a fused, bridged or spiro ring system, and can be saturated or can be partially unsaturated. Heterocyclyl polycyclic ring systems can include one or more heteroatoms in one or both rings. “Heterocyclyl” also includes ring systems wherein the heterocyclyl group, as defined above, is fused with one or more carbocyclyl groups wherein the point of attachment is either on the carbocyclyl or heterocyclyl ring, and in such instances, the number of ring members designates the total number of ring members in the entire ring system. When substitution is indicated in such instances, unless otherwise specified, substitution can occur on either the heterocyclyl or the one or more carbocyclyl groups. Unless otherwise specified, each instance of heterocyclyl is independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3- to 12-membered heterocyclyl. In certain embodiments, the heterocyclyl group is substituted 3- to 12-membered heterocyclyl.

“Fused heterocyclyl” or “fused heterocycle” refers to ring systems wherein the heterocyclyl group, as defined above, is fused with, i.e., share two common atoms (as such, share one common bond) with, one or more heterocyclyl or carbocyclyl groups, as defined above, wherein the point of attachment is on any of the fused rings. In such instances, the number of ring members designates the total number of ring members in the fused ring system. When substitution is indicated, unless otherwise specified, substitution can occur on any of the fused rings.

“Spiro heterocyclyl” or “spiro heterocycle” refers to ring systems wherein the heterocyclyl group, as defined above, form spiro structure with, i.e., share one common atom with, one or more heterocyclyl or carbocyclyl groups, as defined above, wherein the point of attachment is on the heterocyclyl or carbocyclyl rings in which the spiro structure is embedded. In such instances, the number of ring members designates the total number of ring members of the heterocyclyl or carbocyclyl rings in which the spiro structure is embedded. When substitution is indicated, unless otherwise specified, substitution can occur on any of the heterocyclyl or carbocyclyl rings in which the spiro structure is embedded.

“Bridged heterocyclyl” or “bridged heterocycle” refers to ring systems wherein the heterocyclyl group, as defined above, form bridged structure with, i.e., share more than two atoms (as such, share more than one bonds) with, one or more heterocyclyl or carbocyclyl groups, as defined above, wherein the point of attachment is on the heterocyclyl or carbocyclyl rings in which the bridged structure is embedded. In such instances, the number of ring members designates the total number of ring members of the heterocyclyl or carbocyclyl rings in which the bridged structure is embedded. When substitution is indicated, unless otherwise specified, substitution can occur on any of the heterocyclyl or carbocyclyl rings in which the bridged structure is embedded.

“Alkoxy” as used herein, refers to the group —OR, wherein R is alkyl as defined herein. C₁-C₆ alkoxy refers to the group —OR, wherein each R is C₁-C₆ alkyl, as defined herein. Exemplary C₁-C₆ alkyl is set forth above.

“Alkylamino” as used herein, refers to the group —NHR or —NR₂, wherein each R is independently alkyl, as defined herein. C₁-C₆ alkylamino refers to the group —NHR or —NR₂, wherein each R is independently C₁-C₆ alkyl, as defined herein. Exemplary C₁-C₆ alkyl is set forth above.

“Oxo” refers to ═O. When a group other than aryl and heteroaryl or an atom is substituted with an oxo, it is meant to indicate that two geminal radicals on that group or atom form a double bond with an oxygen radical. When a heteroaryl is substituted with an oxo, it is meant to indicate that a resonance structure/tautomer involving a heteroatom provides a carbon atom that is able to form two geminal radicals, which form a double bond with an oxygen radical.

“Halo” or “halogen” refers to fluoro (F), chloro (Cl), bromo (Br), and iodo (I). In certain embodiments, the halo group is either fluoro or chloro.

“Protecting group” as used herein is art-recognized and refers to a chemical moiety introduced into a molecule by chemical modification of a functional group (e.g., hydroxyl, amino, thio, and carboxylic acid) to obtain chemoselectivity in a subsequent chemical reaction, during which the unmodified functional group may not survive or may interfere with the chemical reaction. Common functional groups that need to be protected include but not limited to hydroxyl, amino, thiol, and carboxylic acid. Accordingly, the protecting groups are termed hydroxyl-protecting groups, amino-protecting groups, thiol-protecting groups, and carboxylic acid-protecting groups, respectively.

Common types of hydroxyl-protecting groups include but not limited to ethers (e.g., methoxymethyl (MOM), β-Methoxyethoxymethyl (MEM), tetrahydropyranyl (THP), p-methoxyphenyl (PMP), t-butyl, triphenylmethyl (Trityl), allyl, and benzyl ether (Bn)), silyl ethers (e.g., t-butyldiphenylsilyl (TBDPS), trimethylsilyl (TMS), triisopropylsilyl (TIPS), tri-iso-propylsilyloxymethyl (TOM), and t-butyldimethylsilyl (TBDMS)), and esters (e.g., pivalic acid ester (Piv) and benzoic acid ester (benzoate; Bz)).

Common types of amino-protecting groups include but not limited to carbamates (e.g., t-butyloxycarbonyl (Boc), 9-fluorenylmethyloxycarbonyl (Fmoc), p-methoxybenzyl carbonyl (Moz or MeOZ), 2,2,2-trichloroethoxycarbonyl (Troc), and benzyl carbamate (Cbz)), esters (e.g., acetyl (Ac); benzoyl (Bz), trifluoroacetyl, and phthalimide), amines (e.g., benzyl (Bn), p-methoxybenzyl (PMB), p-methoxyphenyl (PMP), and triphenylmethyl (trityl)), and sulfonamides (e.g., tosyl (Ts), N-alkyl nitrobenzenesulfonamides (Nosyl), and 2-nitrophenylsulfenyl (Nps)).

Common types of thiol-protecting groups include but not limited to sulfide (e.g., p-methylbenzyl (Meb), t-butyl, acetamidomethyl (Acm), and triphenylmethyl (Trityl)).

Common types of carboxylic acid-protecting groups include but not limited to esters (e.g., methyl ester, triphenylmethyl (Trityl), t-butyl ester, benzyl ester (Bn), S-t-butyl ester, silyl esters, and orthoesters) and oxazoline.

These and other exemplary substituents are described in more detail in the Detailed Description, Examples, and claims. The invention is not intended to be limited in any manner by the above exemplary listing of substituents.

### Other Definitions

“Antibody” is used in the broadest sense and specifically encompasses monoclonal antibodies (including full length monoclonal antibodies), polyclonal antibodies, multispecific antibodies (e.g., bispecific antibodies), and antibody fragments so long as they exhibit the desired biological activity. “Antibody fragment” and all grammatical variants thereof as used herein are defined as a portion of an intact antibody comprising the antigen binding site or variable region of the intact antibody, wherein the portion is free of the constant heavy chain domains (i.e., CH₂, CH₃, and CH₄, depending on antibody isotype) of the Fc region of the intact antibody. Examples of antibody fragments include Fab, Fab, Fab′-SH, F(ab′)₂, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv (scFv) molecules; (2) single chain polypeptides containing only one light chain variable domain, or a fragment thereof that contains the three CDRs of the light chain variable domain, without an associated heavy chain moiety; (3) single chain polypeptides containing only one heavy chain variable region, or a fragment thereof containing the three CDRs of the heavy chain variable region, without an associated light chain moiety; (4) nanobodies comprising single Ig domains from non-human species or other specific single-domain binding modules; and (5) multispecific or multivalent structures formed from antibody fragments. In an antibody fragment comprising one or more heavy chains, the heavy chain(s) can contain any constant domain sequence (e.g., CH1 in the IgG isotype) found in a non-Fc region of an intact antibody, and/or can contain any hinge region sequence found in an intact antibody, and/or can contain a leucine zipper sequence fused to or situated in the hinge region sequence or the constant domain sequence of the heavy chain(s). “Antibody” refers to a polypeptide comprising an antigen binding region (including the complementarity determining region (CDRs)) from an immunoglobulin gene or fragments thereof. The term “antibody” specifically encompasses monoclonal antibodies (including full length monoclonal antibodies), polyclonal antibodies, multispecific antibodies (e.g., bispecific antibodies), and antibody fragments that exhibit the desired biological activity. An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa) connected by disulfide bonds. Each chain is composed of structural domains, which are referred to as immunoglobulin domains. These domains are classified into different categories by size and function, e.g., variable domains or regions on the light and heavy chains (VL and VH, respectively) and constant domains or regions on the light and heavy chains (C_{L} and C_{H}, respectively). The N-terminus of each chain defines a variable region of about 100 to 110 or more amino acids, referred to as the paratope, primarily responsible for antigen recognition, i.e., the antigen binding domain. Light chains are classified as either kappa or lambda. Heavy chains are classified as gamma, mu, alpha, delta, or epsilon, which in turn define the immunoglobulin classes, IgG, IgM, IgA, IgD and IgE, respectively. IgG antibodies are large molecules of about 150 kDa composed of four peptide chains. IgG antibodies contain two identical class γ heavy chains of about 50 kDa and two identical light chains of about 25 kDa, thus a tetrameric quaternary structure. The two heavy chains are linked to each other and to a light chain each by disulfide bonds. The resulting tetramer has two identical halves, which together form the Y-like shape. Each end of the fork contains an identical antigen binding domain. There are four IgG subclasses (IgG1, IgG2, IgG3, and IgG4) in humans, named in order of their abundance in serum (i.e., IgG1 is the most abundant). Typically, the antigen binding domain of an antibody will be most critical in specificity and affinity of binding to cancer cells. An antibody that targets a particular antigen includes a bispecific or multispecific antibody with at least one antigen binding region that targets the particular antigen. In some embodiments, the targeted monoclonal antibody is a bispecific antibody with at least one antigen binding region that targets tumor cells. “Antibody construct” refers to an antibody or a fusion protein comprising (i) an antigen binding domain and (ii) an Fc domain. In some embodiments, the binding agent is an antigen-binding antibody “fragment,” which is a construct that comprises at least an antigen-binding region of an antibody, alone or with other components that together constitute the antigen-binding construct. Many different types of antibody “fragments” are known in the art, including, for instance, (i) a Fab fragment, which is a monovalent fragment consisting of the VL, VH, C_{L}, and CH₁ domains, (ii) a F(ab′)₂ fragment, which is a bivalent fragment comprising two Fab fragments linked by a disulfide bridge at the hinge region, (iii) a Fv fragment consisting of the VL and VH domains of a single arm of an antibody, (iv) a Fab′ fragment, which results from breaking the disulfide bridge of an F(ab′)₂ fragment using mild reducing conditions, (v) a disulfide-stabilized Fv fragment (dsFv), and (vi) a single chain Fv (scFv), which is a monovalent molecule consisting of the two domains of the Fv fragment (i.e., VL and VH) joined by a synthetic linker which enables the two domains to be synthesized as a single polypeptide chain. The antibody or antibody fragments can be part of a larger construct, for example, a conjugate or fusion construct of the antibody fragment to additional regions. For instance, in some embodiments, the antibody fragment can be fused to an Fc region as described herein. In other embodiments, the antibody fragment (e.g., a Fab or scFv) can be part of a chimeric antigen receptor or chimeric T-cell receptor, for instance, by fusing to a transmembrane domain (optionally with an intervening linker or “stalk” (e.g., hinge region)) and optional intercellular signaling domain. For instance, the antibody fragment can be fused to the gamma and/or delta chains of a T-cell receptor, so as to provide a T-cell receptor like construct that binds TROP2. In yet another embodiment, the antibody fragment is part of a bispecific T-cell engager (BiTEs) comprising a CD1 or CD3 binding domain and linker. “Epitope” means any antigenic determinant or epitopic determinant of an antigen to which an antigen binding domain binds (i.e., at the paratope of the antigen binding domain). Antigenic determinants usually consist of chemically active surface groupings of molecules, such as amino acids or sugar side chains, and usually have specific three dimensional structural characteristics, as well as specific charge characteristics. The terms “Fc receptor” or “FcR” refer to a receptor that binds to the Fc region of an antibody. There are three main classes of Fc receptors: (1) FcγR which bind to IgG, (2) FcαR which binds to IgA, and (3) FcεR which binds to IgE. The FcγR family includes several members, such as FcγI (CD64), FcγRIIA (CD32A), FcγRIIB (CD32B), FcγRIIIA (CD16A), and FcγRIIIB (CD16B). The Fcγ receptors differ in their affinity for IgG and also have different affinities for the IgG subclasses (e.g., IgG1, IgG2, IgG3, and IgG4).

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

“Pharmaceutically acceptable salt” refers to a salt of a compound of the disclosure that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, chlorobenzenesulfonic acid, 2-naphthalenesulfonic acid, 4-toluenesulfonic acid, camphorsulfonic acid, 4-methylbicyclo [2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium potassium, calcium, magnesium, ammonium, tetraalkylammonium, and the like; and when the compound contains a basic functionality, salts of nontoxic organic or inorganic acids, such as hydrochloride, hydrobromide, tartrate, mesylate, acetate, maleate, oxalate and the like.

A “subject” to which administration is contemplated includes, but is not limited to, humans (i.e., a male or female of any age group, e.g., a pediatric subject (e.g, infant, child, adolescent) or an adult subject (e.g., young adult, middle aged adult or senior adult) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal.

An “effective amount” means the amount of a compound that, when administered to a subject for treating or preventing a disease, is sufficient to affect such treatment or prevention. The “effective amount” can vary depending on the compound, the disease and its severity, and the age, weight, etc., of the subject to be treated. A “therapeutically effective amount” refers to the effective amount for therapeutic treatment. A “prophylactically effective amount” refers to the effective amount for prophylactic treatment.

“Preventing,” “prevention,” or “prophylactic treatment” refers to a reduction in risk of acquiring or developing a disease or disorder (i.e., causing at least one of the clinical symptoms of the disease not to develop in a subject not yet exposed to a disease-causing agent, or in a subject who is predisposed to the disease in advance of disease onset).

The term “prophylaxis” is related to “prevention,” and refers to a measure or procedure the purpose of which is to prevent, rather than to treat or cure a disease. Non limiting examples of prophylactic measures may include the administration of vaccines; the administration of low molecular weight heparin to hospital patients at risk for thrombosis due, for example, to immobilization, and the administration of an anti-malarial agent such as chloroquine, in advance of a visit to a geographical region where malaria is endemic or the risk of contracting malaria is high.

“Treating,” “treatment,” or “therapeutic treatment” of any disease or disorder refers, in one embodiment, to ameliorating the disease or disorder (i.e., arresting the disease or reducing the manifestation, extent or severity of at least one of the clinical symptoms thereof). In another embodiment, “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In a further embodiment, “treating” or “treatment” relates to slowing the progression of the disease.

The term “about” when referring to a number or a numerical range means that the number or numerical range referred to is an approximation within experimental variability or within statistical experimental error, and thus the number or numerical range, in some instances, will vary between 1% and 15% of the stated number or numerical range. In certain embodiments, the number or numerical range vary by 1%, 2%, 3%, 4%, 5%, 6%, 7%, 8%, 9%, 10%, 11%, 12%, 13%, 14%, or 15% of the stated number or numerical range. In certain embodiments, the number or numerical range vary by 1%, 2%, 3%, 4%, or 5% of the stated number or numerical range. In certain embodiments, the number or numerical range vary by 1%, 2%, or 3% of the stated number or numerical range.

The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B,” when used in conjunction with open-ended language such as “comprising” may refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) may refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

The claims should not be read as limited to the described order or elements unless stated to that effect. It should be understood that various changes in form and detail may be made by one of ordinary skill in the art without departing from the spirit and scope of the appended claims. All embodiments that come within the spirit and scope of the following claims and equivalents thereto are claimed.

### EXEMPLARY EMBODIMENTS

Exemplary Embodiment No. 1. A compound of Formula I:

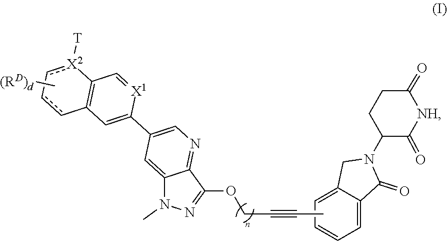

or a pharmaceutically acceptable salt, solvate, or stereoisomer thereof, wherein:
each [symbol] independently is a single bond or a double bond;
X¹ is N or CR^{X1};
R^{X1} is hydrogen or C₁-C₆ alkyl, wherein the C₁-C₆ alkyl is optionally substituted with one or more halogen, —CN, —OH, or —NH₂;
X² is N or C, as valency permits;
T is T-1 or T-2:

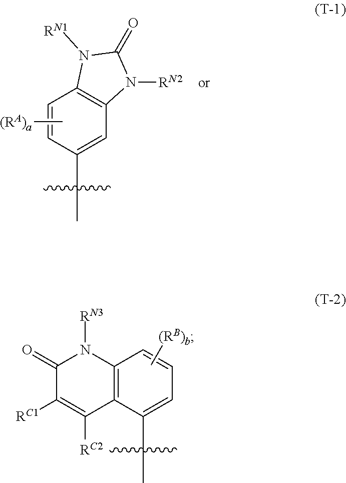

R^{N1} is hydrogen or C₁-C₆ alkyl, wherein the C₁-C₆ alkyl is optionally substituted with one or more halogen, —CN, —OH, or —NH₂;
R^{N2} is hydrogen or C₁-C₆ alkyl, wherein the C₁-C₆ alkyl is optionally substituted with one or more halogen, —CN, —OH, or —NH₂;
R^{N3} is hydrogen or C₁-C₆ alkyl, wherein the C₁-C₆ alkyl is optionally substituted with one or more halogen, —CN, —OH, or —NH₂;
each R^{A} independently is halogen, —CN, —OH, —NH₂, or C₁-C₆ alkyl, wherein the C₁-C₆ alkyl is optionally substituted with one or more halogen, —CN, —OH, or —NH₂;
a is an integer selected from 0 to 3;
each R^{B} independently is halogen, —CN, —OH, —NH₂, or C₁-C₆ alkyl, wherein the C₁-C₆ alkyl is optionally substituted with one or more halogen, —CN, —OH, or —NH₂;
b is an integer selected from 0 to 3;
R^{C1} is hydrogen or C₁-C₆ alkyl, wherein the C₁-C₆ alkyl is optionally substituted with one or more halogen, —CN, —OH, or —NH₂;
R^{C2} is hydrogen or C₁-C₆ alkyl, wherein the C₁-C₆ alkyl is optionally substituted with one or more halogen, —CN, —OH, or —NH₂;
each R^{D} independently is halogen, —CN, —OH, —NH₂, or C₁-C₆ alkyl, wherein the C₁-C₆ alkyl is optionally substituted with one or more halogen, —CN, —OH, or —NH₂;
d is an integer selected from 0 to 5, as valency permits; and
n is an integer selected from 1 to 5.

Exemplary Embodiment No. 2. The compound of Exemplary Embodiment No. 1, wherein the compound is of Formula I-A:

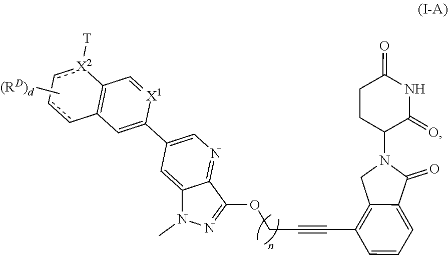

or a pharmaceutically acceptable salt, solvate, or stereoisomer thereof.

Exemplary Embodiment No. 3. The compound of Exemplary Embodiment No. 1 or Exemplary Embodiment No. 2, wherein T is T-1.

Exemplary Embodiment No. 4. The compound of any one of Exemplary Embodiment Nos. 1-3, wherein a is 1.

Exemplary Embodiment No. 5. The compound of any one of Exemplary Embodiment Nos. 1-4, wherein T is T-1-a:

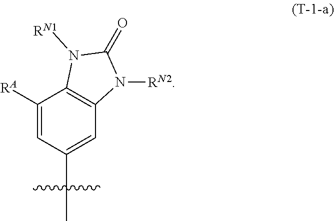

Exemplary Embodiment No. 6. The compound of any one of Exemplary Embodiment Nos. 1-5, wherein R^{N1} is hydrogen.

Exemplary Embodiment No. 7. The compound of any one of Exemplary Embodiment Nos. 1-5, wherein R^{N1} is C₁-C₆ alkyl optionally substituted with one or more halogen, —CN, —OH, or —NH₂.

Exemplary Embodiment No. 8. The compound of Exemplary Embodiment No. 7, wherein R^{N1} is —CH₃.

Exemplary Embodiment No. 9. The compound of any one of Exemplary Embodiment Nos. 1-8, wherein R^{N2} is hydrogen.

Exemplary Embodiment No. 10. The compound of any one of Exemplary Embodiment Nos. 1-8, wherein R^{N2} is C₁-C₆ alkyl optionally substituted with one or more halogen, —CN, —OH, or —NH₂.

Exemplary Embodiment No. 11. The compound of Exemplary Embodiment No. 10, wherein R^{N2} is —CH₃.

Exemplary Embodiment No. 12. The compound of any one of Exemplary Embodiment Nos. 1-11, wherein each R^{A} independently is halogen, —CN, —OH, or —NH₂.

Exemplary Embodiment No. 13. The compound of any one of Exemplary Embodiment Nos. 1-11, wherein each R^{A} independently is C₁-C₆ alkyl optionally substituted with one or more halogen, —CN, —OH, or —NH₂.

Exemplary Embodiment No. 14. The compound of Exemplary Embodiment No. 13, wherein each R^{A} independently is —CH(CH₃)₂.

Exemplary Embodiment No. 15. The compound of Exemplary Embodiment No. 13 or Exemplary Embodiment No. 14, wherein T is

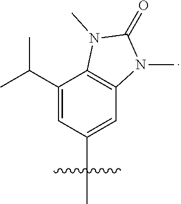 [SMILES: *c1cc(C(C)C)c2c(c1)n(C)c(=O)n2C]

Exemplary Embodiment No. 16. The compound of Exemplary Embodiment No. 1 or Exemplary Embodiment No. 2, wherein T is T-2.

Exemplary Embodiment No. 17. The compound of any one of Exemplary Embodiment Nos. 1, 2, and 16, wherein b is 1.

Exemplary Embodiment No. 18. The compound of any one of Exemplary Embodiment Nos. 1, 2, 16, and 17, wherein T is T-2-a:

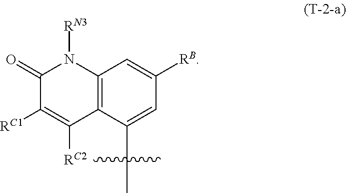

Exemplary Embodiment No. 19. The compound of any one of Exemplary Embodiment Nos. 1, 2, and 16-18, wherein R^{N3} is hydrogen.

Exemplary Embodiment No. 20. The compound of any one of Exemplary Embodiment Nos. 1, 2, and 16-18, wherein R^{N3} is C₁-C₆ alkyl optionally substituted with one or more halogen, —CN, —OH, or —NH₂.

Exemplary Embodiment No. 21. The compound of Exemplary Embodiment No. 20, wherein R^{N3} is —CH₃.

Exemplary Embodiment No. 22. The compound of any one of Exemplary Embodiment Nos. 1, 2, and 16-21, wherein R^{C1} is hydrogen.

Exemplary Embodiment No. 23. The compound of any one of Exemplary Embodiment Nos. 1, 2, and 16-21, wherein R^{C1} is C₁-C₆ alkyl optionally substituted with one or more halogen, —CN, —OH, or —NH₂.

Exemplary Embodiment No. 24. The compound of Exemplary Embodiment No. 23, wherein Rei is —CH₃.

Exemplary Embodiment No. 25. The compound of any one of Exemplary Embodiment Nos. 1, 2, and 16-24, wherein R^{C2} is hydrogen.

Exemplary Embodiment No. 26. The compound of any one of Exemplary Embodiment Nos. 1, 2, and 16-24, wherein R^{C2} is C₁-C₆ alkyl optionally substituted with one or more halogen, —CN, —OH, or —NH₂.

Exemplary Embodiment No. 27. The compound of any one of Exemplary Embodiment Nos. 1, 2, and 16-26, wherein each R^{B} independently is halogen, —CN, —OH, or —NH₂.

Exemplary Embodiment No. 28. The compound of any one of Exemplary Embodiment Nos. 1, 2, and 16-26, wherein each R^{B} independently is C₁-C₆ alkyl optionally substituted with one or more halogen, —CN, —OH, or —NH₂.

Exemplary Embodiment No. 29. The compound of Exemplary Embodiment No. 28, wherein each R^{B} independently is —CH₂(CH₃).

Exemplary Embodiment No. 30. The compound of Exemplary Embodiment No. 28 or Exemplary Embodiment No. 29, wherein T is

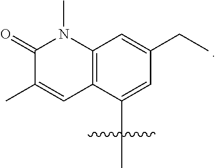 [SMILES: *c1cc(CC)cc2c1cc(C)c(=O)n2C]

Exemplary Embodiment No. 31. The compound of any one of Exemplary Embodiment Nos. 1-30, wherein X¹ is N.

Exemplary Embodiment No. 32. The compound of any one of Exemplary Embodiment Nos. 1-30, wherein X¹ is CR^{X1}.

Exemplary Embodiment No. 33. The compound of any one of Exemplary Embodiment Nos. 1-30 and 32, wherein R^{X1} is hydrogen.

Exemplary Embodiment No. 34. The compound of any one of Exemplary Embodiment Nos. 1-30 and 32, wherein R^{X1} is C₁-C₆ alkyl optionally substituted with one or more halogen, —CN, —OH, or —NH₂.

Exemplary Embodiment No. 35. The compound of Exemplary Embodiment No. 34, wherein R^{X1} is —CHF₂.

Exemplary Embodiment No. 36. The compound of any one of Exemplary Embodiment Nos. 1-35, wherein X² is N.

Exemplary Embodiment No. 37. The compound of any one of Exemplary Embodiment Nos. 1-35, wherein X² is C.

Exemplary Embodiment No. 38. The compound of any one of Exemplary Embodiment Nos. 1-37, wherein each R^{D} independently is halogen, —CN, —OH, or —NH₂.

Exemplary Embodiment No. 39. The compound of any one of Exemplary Embodiment Nos. 1-37, wherein each R^{D} independently is C₁-C₆ alkyl optionally substituted with one or more halogen, —CN, —OH, or —NH₂.

Exemplary Embodiment No. 40. The compound of any one of Exemplary Embodiment Nos. 1-39, wherein d is 0.

Exemplary Embodiment No. 41. The compound of any one of Exemplary Embodiment Nos. 1-40, wherein n is an integer selected from 1 to 3.

Exemplary Embodiment No. 42. A compound selected from the compounds in Table 1 or a pharmaceutically acceptable salt, solvate, or stereoisomer thereof.

Exemplary Embodiment No. 43. A pharmaceutical composition comprising the compound of any one of Exemplary Embodiment Nos. 1-42 and one or more pharmaceutically acceptable excipient.

Exemplary Embodiment No. 44. A method of degrading a protein in a subject or biological sample comprising administering the compound of any one of Exemplary Embodiment Nos. 1-42 to the subject or contacting the biological sample with the compound of any one of Exemplary Embodiment Nos. 1-42.

Exemplary Embodiment No. 45. Use of the compound of any one of Exemplary Embodiment Nos. 1-42 in the manufacture of a medicament for degrading a protein in a subject or biological sample.

Exemplary Embodiment No. 46. A compound of any one of Exemplary Embodiment Nos. 1-42 for use in degrading a protein in a subject or biological sample.

Exemplary Embodiment No. 47. The method, use, or compound for use of any one of Exemplary Embodiment Nos. 44-46, wherein the protein is p300 or CBP.

Exemplary Embodiment No. 48. A method of treating a p300-mediated disorder comprising administering to a patient in need thereof a compound of any one of Exemplary Embodiment Nos. 1-42.

Exemplary Embodiment No. 49. Use of a compound of any one of Exemplary Embodiment Nos. 1-42 in the manufacture of a medicament for treating a p300-mediated disorder.

Exemplary Embodiment No. 50. A compound of any one of Exemplary Embodiment Nos. 1-42 for use in treating a p300-mediated disorder.

Exemplary Embodiment No. 51. The method, use, or compound for use of any one of Exemplary Embodiment Nos. 48-50, wherein the p300-mediated disorder is cancer, an inflammatory disorder, or an autoimmune disease.

### EXAMPLES

In order that the invention described herein may be more fully understood, the following examples are set forth. The examples described in this application are offered to illustrate the compounds, pharmaceutical compositions, and methods provided herein and are not to be construed in any way as limiting their scope.

### I. Synthesis and Characterization

In the following examples, the chemical reagents were purchased from commercial sources (such as Alfa, Acros, Sigma Aldrich, TCI and Shanghai Chemical Reagent Company), and used without further purification.

In obtaining the compounds described in the examples below and the corresponding analytical data, the following experimental and analytical protocols were followed unless otherwise indicated.

Unless otherwise stated, reaction mixtures were magnetically stirred at room temperature (rt) under a nitrogen atmosphere. Where solutions were “dried,” they were generally dried over a drying agent such as Na₂SO₄ or MgSO₄. Where mixtures, solutions, and extracts were “concentrated,” they were typically concentrated on a rotary evaporator under reduced pressure.

Compound purification was carried out as needed using a variety of traditional methods including, but not limited to, preparative chromatography under acidic, neutral, or basic conditions using either normal phase or reverse phase HPLC or flash columns or Prep-TLC plates.

Flash chromatography was performed on a Biotage Isolera One via column with silica gel particles of 100-200 mesh or 200-300 mesh. Analytical and preparative thin-layer chromatography was performed using silica gel 60 GF254 plates. Normal-phase silica gel chromatography (FCC) was also performed on silica gel (SiO₂) using prepacked cartridges.

Preparative reverse-phase high performance liquid chromatography (RP HPLC) was performed on either:

### Method A

Prep-HPLC with YMC-Actus Triart 18C (5 μm, 20×250 mm), and mobile phase of 5-99% ACN in water (0.1% HCOOH) over 10 min and then hold at 100% ACN for 2 min, at a flow rate of 25 mL/min; or

### Method B

Preparative supercritical fluid high performance liquid chromatography (SFC) was performed either on a Thar 80 Prep-SFC system, or Waters 80Q Prep-SFC system from Waters. The ABPR was set to 100 bar to keep the CO₂ in SF conditions, and the flow rate may verify according to the compound characteristics, with a flow rate ranging from 50 g/min to 70 g/min. The column temperature was ambient temperature.

Nuclear magnetic resonance (NMR) spectra were recorded using Brucker AVANCE NEO 400 MHz at around 20-30° C. unless otherwise specified. The following abbreviations are used: s, singlet; d, doublet; t, triplet; q, quartet; m, multiplet; dd, doublet of doublets; ddd, doublet of doublet of doublets; dt, doublet of triplets; bs, broad signal. Chemical shifts were reported in parts per million (ppm, δ) downfield from tetramethylsilane. It will be understood that for compounds comprising an exchangeable proton, said proton may or may not be visible on an NMR spectrum depending on the choice of solvent used for running the NMR spectrum and the concentration of the compound in the solution.

Mass spectra (MS) were obtained on a SHIMADZU LCMS-2020 MSD using electrospray ionization (ESI) in positive mode unless otherwise indicated. Calculated (calcd.) mass corresponds to the exact mass.

Chemical names were generated using ChemDraw Ultra 12.0, ChemDraw Ultra 14.0, ChemDraw Ultra 20.0 (CambridgeSoft Corp., Cambridge, MA) or ACD/Name Version 10.01 (Advanced Chemistry).

### Example 1: 3-(4-(4-((6-(7-(difluoromethyl)-7′-ethyl-1′,3′-dimethyl-2′-oxo-1′,2′,3,4-tetrahydro-2H-[1,5′-biquinolin]-6-yl)-1-methyl-1H-pyrazolo[4,3-b]pyridin-3-yl)oxy)but-1-yn-1-yl)-1-oxoisoindolin-2-yl)piperidine-2,6-dione

Step A: 5-Bromo-7-hydroxy-1,3-dimethylquinolin-2(1H)-one

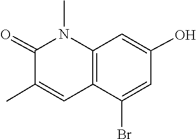

To a solution of 5-bromo-7-methoxy-1,3-dimethylquinolin-2(1H)-one (20.0 g, 70.9 mmol, 1 eq.) in DCM (200 mL) was added BBr₃ (55 mL, 567 mmol, 8 eq.). The mixture was stirred at 25° C. for 2 hour under N₂. The resulting solution was quenched with MeOH and filtered to give 5-bromo-7-hydroxy-1,3-dimethylquinolin-2(1H)-one (18.0 g, 67.1 mmol, 95% yield) as a white solid.

LC-MS (ESI): mass calcd. for C₁₁H₁₀BrNO₂, 267.0/269.0. m/z found, 268.1/270.1 [M+H]⁺.

### Step B: 5-Bromo-1,3-dimethyl-2-oxo-1,2-dihydroquinolin-7-yl trifluoromethanesulfonate

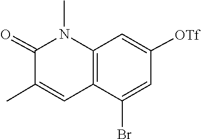 [SMILES: Cc1cc2c(Br)cc(OS(=O)(=O)C(F)(F)F)cc2n(C)c1=O]

To a mixture of 5-bromo-7-hydroxy-1,3-dimethylquinolin-2(1H)-one (16.0 g, 60 mmol, 1 eq.) and pyridine (14.5 mL, 179 mmol, 3 eq.) in DCM (160 mL) was added triflic anhydride (20 mL, 119 mmol, 2 eq.) at 0° C. under N₂ atmosphere. The mixture was stirred at 0° C. for 3 hours. The resulting solution was diluted with 200 mL of water and extracted with EtOAc (200 mL×3). The combined organic layers were washed with brine (200 mL), dried over Na₂SO₄, and concentrated under reduced pressure. The residue was purified by flash column chromatography on silica gel (100-200 mesh silica gel, 0-25% EtOAc in PE) to give 5-bromo-1,3-dimethyl-2-oxo-1,2-dihydroquinolin-7-yl trifluoromethanesulfonate (19.0 g, 47.5 mmol, 80% yield) as a yellow solid.

LC-MS (ESI): mass calcd. for C₁₂H₉BrF₃NO₄S, 398.9/400.9. m/z found, 400/402 [M+H]⁺.

### Step C: 5-Bromo-1,3-dimethyl-7-vinylquinolin-2(1H)-one

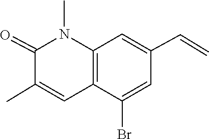 [SMILES: C=Cc1cc(Br)c2cc(C)c(=O)n(C)c2c1]

To a stirred mixture of 5-bromo-1,3-dimethyl-2-oxo-1,2-dihydroquinolin-7-yl trifluoromethanesulfonate (10.0 g, 25 mmol, 1 eq.) and potassium vinyltrifluoroborate (3.8 g, 28.7 mmol, 1.15 eq.) in THF (80 mL) and water (6 mL) was added PdCl₂(dppf) (1.8 g, 2.5 mmol, 0.1 eq.) and K₂CO₃ (10.4 g, 75 mmol, 3 eq.) at room temperature under N₂ atmosphere. The reaction mixture was stirred at 50° C. for 3 hours. The mixture was diluted with water and extracted with EtOAc. The combined organic layers were washed with brine, dried over Na₂SO₄, and concentrated under reduced pressure. The residue was purified by flash column chromatography (50% EtOAc in PE) to give 5-bromo-1,3-dimethyl-7-vinylquinolin-2(1H)-one (5.0 g, 14 mmol, 58% yield, 80% Purity) as a white solid.

LC-MS (ESI): mass calcd. for C₁₃H₁₂BrNO, 277.0/279.0. m/z found, 278.0/280.0 [M+H]⁺.

### Step D: 5-Bromo-7-ethyl-1,3-dimethylquinolin-2(1H)-one

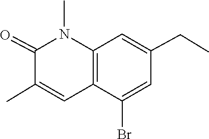 [SMILES: CCc1cc(Br)c2cc(C)c(=O)n(C)c2c1]

To a solution of 5-bromo-1,3-dimethyl-7-vinylquinolin-2(1H)-one (5.0 g, 80% wt., 14.4 mmol, 1 eq.) in EtOH (40 mL) was added Wilkinson's catalyst (1.5 g, 1.62 mmol, 0.11 eq.) at room temperature. The reaction mixture was stirred at 50° C. under H2 atmosphere for 4 hours. The mixture was concentrated under reduced pressure. The residue was purified by flash chromatography (20% EtOAc in DCM) to give 5-bromo-7-ethyl-1,3-dimethylquinolin-2(1H)-one (3.0 g, 10.7 mmol, 75% yield).

LC-MS (ESI): mass calcd. for C₁₃H₁₄BrNO, 279.0/281.0. m/z found, 280/282 [M+H]⁺.

### Step E: Quinoline-7-carbaldehyde

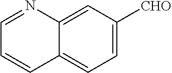 [SMILES: O=Cc1ccc2cccnc2c1]

To a mixture of 7-methylquinoline (25 g, 175 mmol) was added SeO₂ (38.7 g, 349.2 mmol) in portions over 5 min at room temperature. The mixture was stirred at 160° C. for 3 hours under N₂ atmosphere. The cooled mixture was diluted with DCM (200 mL) and filtered. The filtrate was concentrated under reduced pressure. The residue was purified by flash chromatography (0-10% EtOAc in PE) to give quinoline-7-carbaldehyde (13 g, 47% yield) as a yellow solid.

LC-MS (ESI): mass calcd. for C₁₀H₇NO: 157. m/z found: 158 [M+H]⁺.

### Step F: 7-(Difluoromethyl)quinoline

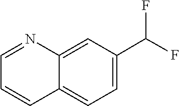 [SMILES: FC(F)c1ccc2cccnc2c1]

To a stirred mixture of quinoline-7-carbaldehyde (5 g, 31.8 mmol) in DCM (10 mL) was added DAST (12.6 mL, 95.4 mmol) dropwise at −20° C. The reaction mixture was stirred overnight at room temperature. The mixture was poured into aq. NaHCO₃ at 0° C. and extracted with DCM (30 mL×3). The combined organic layers were washed with brine, dried over anhydrous Na₂SO₄, and concentrated under reduced pressure. The residue was purified by flash chromatography (0-10% EtOAc in PE) to give 7-(difluoromethyl)quinoline (3.6 g, 63% yield) as a yellow solid.

LC-MS (ESI): mass calcd. for C₁₀H₇F₂N: 179. m/z found: 180 [M+H]⁺.

### Step G: 7-(Difluoromethyl)-1,2,3,4-tetrahydroquinoline

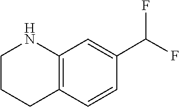 [SMILES: FC(F)c1ccc2c(c1)NCCC2]

To a solution of 7-(difluoromethyl)quinoline (5 g, 27.9 mmol) and NaBH₃CN (9.0 g, 140 mmol) in MeOH (100 mL) was added BF₃·Et₂O (6.9 mL, 55.8 mmol) dropwise at 0° C. The reaction mixture was stirred overnight at 90° C. under N₂ atmosphere. The mixture was poured into saturated aq. NaHCO₃ (50 mL) and extracted with DCM (30 mL×3). The combined organic layers were washed with brine, dried over anhydrous Na₂SO₄, and concentrated under reduced pressure. The residue was purified by flash chromatography (0-10% EtOAc in PE) to give 7-(difluoromethyl)-1,2,3,4-tetrahydroquinoline (3.6 g, 69% yield) as a brown oil.

LC-MS (ESI): mass calcd. for C₁₀H₁₁F₂N: 183. m/z found: 184 [M+H]⁺.

### Step H: 6-Chloro-7-(difluoromethyl)-1,2,3,4-tetrahydroquinoline

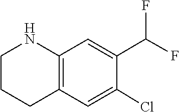 [SMILES: FC(F)c1cc2c(cc1Cl)CCCN2]

To a solution of 7-(difluoromethyl)-1,2,3,4-tetrahydroquinoline (2.33 g, 12.7 mmol) in ACN (20.0 mL) was added with NCS (1.70 g, 12.7 mmol) at 0° C. The mixture was stirred at 16 hours at room temperature. The mixture was diluted with DCM, washed with brine, dried over anhydrous Na₂SO₄, concentrated. The crude product was purified by silica gel chromatograph (eluted with EtOAc in petroleum ether from 10% to 90%) to give 8-chloro-7-(difluoromethyl)-1,2,3,4-tetrahydroquinoline (605 mg, 2.78 mmol, 21.9%) and 6-chloro-7-(difluoromethyl)-1,2,3,4-tetrahydroquinoline (900 mg, 4.14 mmol, 32.5%) as yellow oil.

LC-MS (ESI): mass calcd. for C₁₀H₁₀ClF₂N: 217.1. m/z found: 218.2 [M+H]⁺.

### Step I: 6-chloro-7-(difluoromethyl)-7′-ethyl-1′,3′-dimethyl-3,4-dihydro-2H-[1,5′-biquinolin]-2′(1′H)-one

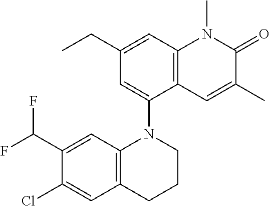 [SMILES: CCc1cc(N2CCCc3cc(Cl)c(C(F)F)cc32)c2cc(C)c(=O)n(C)c2c1]

To a solution of 6-chloro-7-(difluoromethyl)-1,2,3,4-tetrahydroquinoline (400 mg, 1.84 mmol), 7-5-bromo-1,3-dimethyl-7-vinylquinolin-2(1H)-one (1.93 g, 5.51 mmol), Cs₂CO₃ (599 mg, 1.84 mmol), xantphos (213 mg, 368 μmol), Pd₂(dba)₃ (168 mg, 184 μmol) in 1,4-dioxane (10.0 mL) was stirred at 110° C. for 15 hrs under N₂. The mixture was filtered, the crude product was purified by silica gel chromatography (PE:EA=10:1). The product 6-chloro-7-(difluoromethyl)-7′-ethyl-1′,3′-dimethyl-3,4-dihydro-2H-[1,5′-biquinolin]-2′(1′H)-one (610 mg, 1.46 mmol, 79.6%) was obtained as a white solid.

LC-MS (ESI): mass calcd. for C₂₃H₂₃ClF₂N₂O: 416.9. m/z found: 417.6 [M+H]⁺.

### Step J: 7-(difluoromethyl)-7′-ethyl-1′,3′-dimethyl-6-(4,4,5,5-tetramethyl-1,3,2-dioxaborolan-2-yl)-3,4-dihydro-2H-[1,5′-biquinolin]-2′(1′H)-one

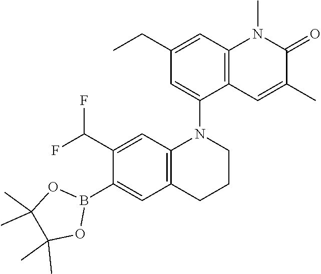

To a solution of 6-chloro-7-(difluoromethyl)-7′-ethyl-1′,3′-dimethyl-3,4-dihydro-2H-[1,5′-biquinolin]-2′(1′H)-one (1.117 g, 2.679 mmol) in 1,4-dioxane (10.0 mL) was added with Cs2CO3 (2.619 g, 8.038 mmol), bis(pinacolato)diborane (1.361 g, 5.359 mmol) and methanesulfonato{[4-(N,N-dimethylamino)phenyl]di-t-butylphosphino}(2′-amino-1,1′-biphenyl-2-yl)palladium (II) (511.3 mg, 803.8 μmol) at 25° C. The mixture was stirred at 100° C. for 12 hour. The mixture was diluted with ethyl acetate, washed with brine, dried over anhydrous Na₂SO₄, concentrated. The crude product was purified by Prep-TLC (PE:EA=4:1) to give 7-(difluoromethyl)-7′-ethyl-1′,3′-dimethyl-6-(4,4,5,5-tetramethyl-1,3,2-dioxaborolan-2-yl)-3,4-dihydro-2H-[1,5′-biquinolin]-2′(1′H)-one (760 mg, 0.52 mmol, 20%) as yellow solid.

LC-MS (ESI): mass calcd. for C₂₉H₃₅BF₂N₂O₃: 508.3. m/z found: 509.2 [M+H]⁺.

### Step K: 6-bromo-1-methyl-1H-pyrazolo[4,3-b]pyridin-3-ol

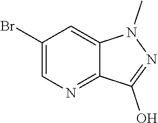

To a stirred solution of methyl 5-bromo-3-fluoropicolinate (800 mg, 3.42 mmol) in 1-butanol (20.0 mL) was added by methylhydrazine (630 mg, 720 μL, 13.7 mmol) at room temperature. The mixture was stirred at 130° C. under N₂ for 4 hours. The mixture was diluted with water and extracted with EtOAc. The combined organic layers were washed with brine, dried over anhydrous Na₂SO₄ and concentrated under reduced pressure. The residue was purified by flash chromatography (eluent, 0˜50% ethyl acetate in petroleum ether) to give 6-bromo-1-methyl-1H-pyrazolo[4,3-b]pyridin-3-ol (200 mg, 877 μmol, 25.7%) as a yellow solid.

LC-MS (ESI): mass calcd. for C₇H₆BrN₃O: 226.9. m/z found: 227.8 [M+H]⁺.

### Step L: 7-(difluoromethyl)-7′-ethyl-6-(3-hydroxy-1-methyl-1H-pyrazolo[4,3-b]pyridin-6-yl)-1′,3′-dimethyl-3,4-dihydro-2H-[1,5′-biquinolin]-2′(1′H)-one

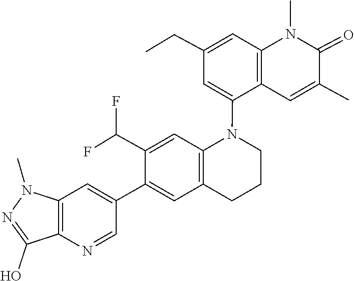 [SMILES: CCc1cc(N2CCCc3cc(-c4cnc5c(O)nn(C)c5c4)c(C(F)F)cc32)c2cc(C)c(=O)n(C)c2c1]

To a solution of 7-(difluoromethyl)-7′-ethyl-6-(3-hydroxy-1-methyl-1H-pyrazolo[4,3-b]pyridin-6-yl)-1′,3′-dimethyl-3,4-dihydro-2H-[1,5′-biquinolin]-2′(1′H)-one (400 mg, 0.67 mmol, 59%) and 6-bromo-1-methyl-1H-pyrazolo[4,3-b]pyridin-3-ol (260 mg, 1.14 mmol) in dioxane (8.00 mL) and H₂O (1.60 mL) was added with potassium phosphate, tribasic (484 mg, 189 μL, 2.28 mmol) and 1,1′-bis(diphenylphosphino)ferrocene-palladium(II) dichloride (83.4 mg, 114 μmol). The mixture was stirred at 100° C. for 3 hours under N₂. The mixture was concentrated to afford a crude product. The crude product was purified by silica gel chromatography (DCM:MeOH=50:1). The product was obtained as a yellow solid 7-(difluoromethyl)-7′-ethyl-6-(3-hydroxy-1-methyl-1H-pyrazolo[4,3-b]pyridin-6-yl)-1′,3′-dimethyl-3,4-dihydro-2H-[1,5′-biquinolin]-2′(1′H)-one (400 mg, 0.67 mmol, 59%).

LC-MS (ESI): mass calcd. for C₃₀H₂₉F₂N₅O₂: 529.2. m/z found: 530.1 [M+H]⁺.

### Step M: 3-(4-(4-hydroxybut-1-yn-1-yl)-1-oxoisoindolin-2-yl)piperidine-2,6-dione

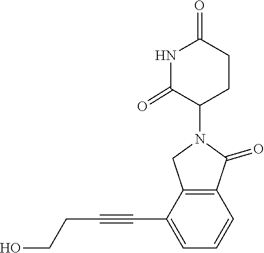 [SMILES: O=C1CCC(N2Cc3c(C#CCCO)cccc3C2=O)C(=O)N1]

To a stirred suspension of 4-bromo-2-(2,6-dioxopiperidin-3-yl)isoindoline-1,3-dione (500 mg, 1.48 mmol) in DMF (3.00 mL) was added cuprous iodide (28.2 mg, 5.03 μL, 148 μmol), bis-(triphenylphosphino)-palladous chloride (104 mg, 148 μmol), TEA (1.00 mL), but-3-yn-1-ol (208 mg, 2.97 mmol) at room temperature and the resulting mixture was stirred for 3 hours under N₂. The reaction mixture was diluted with water (20 mL), extracted with EtOAc (30 mL×3). The combined organic phase was washed by brine, dried over anhydrous Na₂SO₄, filtered and concentrated. The residue was purified by flash chromatography on silica gel (EtOAc in PE=0˜50%) to obtain 2-(2,6-dioxopiperidin-3-yl)-4-(4-hydroxybut-1-yn-1-yl)isoindoline-1,3-dione (475 mg, 1.46 mmol, 98.2%) as yellow oil.

LC-MS (ESI): mass calcd. for C₁₇H₁₆N₂O₄: 312.1. m/z found: 313.1 [M+H]⁺.

### Step N. 3-(4-(4-((6-(7-(difluoromethyl)-7′-ethyl-1′,3′-dimethyl-2′-oxo-1′,2′,3,4-tetrahydro-2H-[1,5′-biquinolin]-6-yl)-I-methyl-1H-pyrazolo[4,3-b]pyridin-3-yl)oxy)but-1-yn-1-yl)-1-oxoisoindolin-2-yl)piperidine-2,6-dione

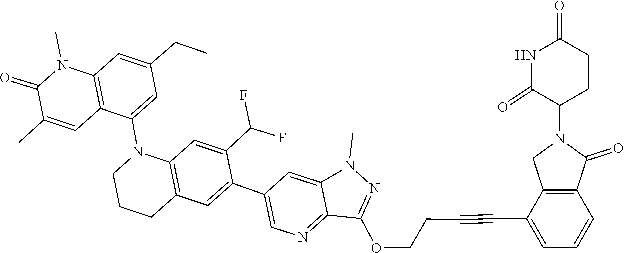 [SMILES: CCc1cc(N2CCCc3cc(-c4cnc5c(OCCC#Cc6cccc7c6CN(C6CCC(=O)NC6=O)C7=O)nn(C)c5c4)c(C(F)F)cc32)c2cc(C)c(=O)n(C)c2c1]

To a solution of 7-(difluoromethyl)-7′-ethyl-6-(3-hydroxy-1-methyl-1H-pyrazolo[4,3-b]pyridin-6-yl)-1′,3′-dimethyl-3,4-dihydro-2H-[1,5′-biquinolin]-2′(1′H)-one (50.0 mg, 94.4 μmol), 3-(4-(4-hydroxybut-1-yn-1-yl)-1-oxoisoindolin-2-yl)piperidine-2,6-dione (29.5 mg, 94.4 μmol), triphenylphosphine (49.5 mg, 41.8 μL, 189 μmol) in DCM (3.00 mL) under N₂, stirred until the reaction system became homogeneous, and then added DIAD (38.2 mg, 36.7 μL, 189 μmol) was stirred at 25° C. for 16 hours under N₂. The mixture was extracted with 50 mL ethyl acetate and 50 mL H₂O, brine, concentrated. The crude product was purified by HPLC with 0.1% FA to get the product 3-(4-(4-((6-(7-(difluoromethyl)-7′-ethyl-1′,3′-dimethyl-2′-oxo-1′,2′,3,4-tetrahydro-2H-[1,5′-biquinolin]-6-yl)-1-methyl-1H-pyrazolo[4,3-b]pyridin-3-yl)oxy)but-1-yn-1-yl)-1-oxoisoindolin-2-yl)piperidine-2,6-dione (4.00 mg, 4.80 μmol, 5.09%, 98.89% purity) as a white solid.

LC-MS (ESI): mass calcd. for C₄₇H₄₃F₂N₇O₅: 823.9. m/z found: 824.7 [M+H]⁺.

¹H NMR (400 MHz, DMSO-d₆) δ 10.9 (s, 1H), 8.35 (s, 1H), 7.92 (s, 1H), 7.79 (d, J=7.5 Hz, 1H), 7.76 (d, J=8.5 Hz, 1H), 7.62 (s, 1H), 7.52 (t, J=9.2 Hz, 1H), 7.45 (s, 1H), 7.15 (d, J=9.5 Hz, 2H), 6.70 (m, J=15.5 Hz, 1H), 6.25 (s, 1H), 5.06 (dd, J=8.0, 6.5 Hz, 1H), 4.68 (t, J=7.5 Hz, 2H), 4.22 (abq, J=11.5, 7.5 Hz, 2H), 3.92 (s, 3H), 3.76 (s, 3H), 3.65 (m, 2H), 3.02 (m, 2H), 2.86 (m, 2H), 2.75 (m, 2H), 1.28 (t, J=9.5 Hz, 3H).

### Example 2: 3-(4-(3-((6-(8-(7-isopropyl-1,3-dimethyl-2-oxo-2,3-dihydro-1H-benzo[d]imidazol-5-yl)isoquinolin-3-yl)-1-methyl-1H-pyrazolo[4,3-b]pyridin-3-yl)oxy)prop-1-yn-1-yl)-1-oxoisoindolin-2-yl)piperidine-2,6-dione

### Step A: 4-isopropyl-1,3-dimethyl-6-(3-((triisopropylsilyl) oxy) isoquinolin-8-yl)-1,3-dihydro-2H-benzo[d]imidazol-2-one

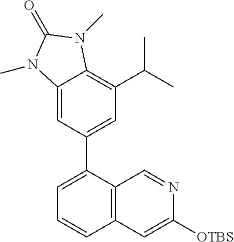 [SMILES: CC(C)c1cc(-c2cccc3cc(O[Si](C)(C)C(C)(C)C)ncc23)cc2c1n(C)c(=O)n2C]

To a solution of 8-(4,4,5,5-tetramethyl-1,3,2-dioxaborolan-2-yl)-3-((triisopropylsilyl)oxy) isoquinoline (1.30 g, 3.04 mmol), xphos palladacycle (239 mg, 304 μmol) and 6-bromo-4-isopropyl-1,3-dimethyl-1,3-dihydro-2H-benzo[d]imidazol-2-one (861 mg, 3.04 mmol) in 1,4-dioxane:H₂O=10:1 (10.0 mL) was added with K₂CO₃ (1.26 g, 9.12 mmol) at 25° C. The mixture was stirred at 100° C. for 3 hours under N₂ atmosphere. The resulting solution was diluted with 20 ml of water, then extracted with EtOAc (3×20 mL) and washed with brine (2×50 mL). The combined organic layers were dried over anhydrous Na₂SO₄ and concentrated under reduced pressure. The residue was purified by flash column chromatography on silica gel (100-200 mesh silica gel, 0-100% EtOAc in PE) to afford 4-isopropyl-1,3-dimethyl-6-(3-((triisopropylsilyl) oxy) isoquinolin-8-yl)-1,3-dihydro-2H-benzo[d]imidazol-2-one (370 mg, 734 μmol, 24.2%) as a brown solid.

LC-MS (ESI): mass calcd. for C₂₇H₃₅N₃O₂Si: 461.3. m/z found: 462.2 [M+H]⁺.

### Step B: 6-(3-hydroxyisoquinolin-8-yl)-4-isopropyl-1,3-dimethyl-1,3-dihydro-2H-benzo[d]imidazol-2-one

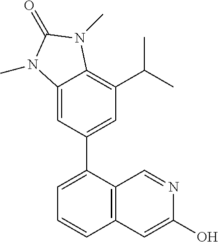 [SMILES: CC(C)c1cc(-c2cccc3cc(O)ncc23)cc2c1n(C)c(=O)n2C]

To a solution of 4-isopropyl-1,3-dimethyl-6-(3-((triisopropylsilyl)oxy)isoquinolin-8-yl)-1,3-dihydro-2H-benzo[d]imidazol-2-one (7.00 g, 13.9 mmol) in MeOH (80.0 mL) was added by ammonium fluoride (1.54 g, 1.54 mL, 41.7 mmol) at 0° C., and then the reaction mixture was stirred at 25° C. for 2 hours. The reaction mixture was concentrated in vacuo to give a residue. The resulting solution was concentrated in vacuo to give a residue. The residue was purified by flash column chromatography on silica gel (100-200 mesh silica gel, 0-10% DCM in MeOH) to give 6-(3-hydroxyisoquinolin-8-yl)-4-isopropyl-1,3-dimethyl-1,3-dihydro-2H-benzo[d]imidazol-2-one (4.00 g, 11.5 mmol, 82.9%) as a solid.

LC-MS (ESI): mass calcd. for C₂₁H₂₀N₃O: 330.2. m/z found: 331.2 [M+H]⁺.

### Step C: 8-(7-isopropyl-1,3-dimethyl-2-oxo-2,3-dihydro-1H-benzo[d]imidazol-5-yl)isoquinolin-3-yl Trifluoromethanesulfonate

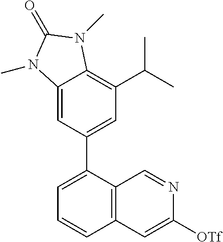 [SMILES: CC(C)c1cc(-c2cccc3cc(OS(=O)(=O)C(F)(F)F)ncc23)cc2c1n(C)c(=O)n2C]

To a solution of 6-(3-hydroxyisoquinolin-8-yl)-4-isopropyl-1,3-dimethyl-1,3-dihydro-2H-benzo[d]imidazol-2-one (4.10 g, 11.8 mmol) and TEA (3.58 g, 4.93 mL, 35.4 mmol) in DCM (80.0 mL) was added phenyl triflimide (6.32 g, 17.7 mmol) at 25° C. The reaction mixture was stirred at 25° C. for 2 hours. The reaction mixture was poured into water and extracted with DCM. The organic layer was washed with H₂O, dried Na₂SO₄ and concentrated to give a residue. The residue was purified by column chromatography on silica gel eluted with PE/EA (100:1-1:1) to give 8-(7-isopropyl-1,3-dimethyl-2-oxo-2,3-dihydro-1H-benzo[d]imidazol-5-yl)isoquinolin-3-yl trifluoromethanesulfonate (4.50 g, 9.39 mmol, 79.5%) as a white solid.

LC-MS (ESI): mass calcd. for C₂₂H₂₀F₃N₃O₄S: 479.1. m/z found: 480.2 [M+H]⁺.

### Step D: 3-((tert-butyldimethylsilyl)oxy)-1-methyl-6-(4,4,5,5-tetramethyl-1,3,2-dioxaborolan-2-yl)-1H-pyrazolo[4,3-b]pyridine

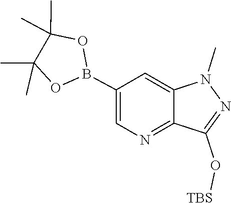

To a solution of 6-bromo-1-methyl-1H-pyrazolo[4,3-b]pyridin-3-ol (1.80 g, 7.89 mmol), imidazole (1.07 g, 1.04 mL, 15.8 mmol) in DCM (15.0 mL) was added TBS-Cl (1.43 g, 9.47 mmol), the mixture was stirred for 1 hour. The resulting solution was diluted with 40 mL of H₂O and extracted with DCM (40 mL×3). The combined organic layers were washed with brine, dried over Na₂SO₄ and concentrated under reduced pressure. The residue was purified by flash column chromatography on silica gel (100-200 mesh silica gel, 0-60% EtOAc in PE) to give 6-bromo-3-((tert-butyldimethylsilyl)oxy)-1-methyl-1H-pyrazolo[4,3-b]pyridine (2.30 g, 6.72 mmol, 85.1%) as a solid.

To a mixture of 6-bromo-3-((tert-butyldimethylsilyl)oxy)-1-methyl-1H-pyrazolo[4,3-b]pyridine (170 mg, 497 μmol), 4,4,4′,4′,5,5,5′,5′-octamethyl-2,2′-bi(1,3,2-dioxaborolane) (151 mg, 596 μmol), PdCl₂(dppf) (36.3 mg, 49.7 μmol), and potassium acetate (146 mg, 1.49 mmol) in 1,4-dioxane (8.00 mL) was stirred at 100° C. under N₂ for 3 hours. The resulting solution was concentrated in vacuo to give a residue. The residue was purified by flash column chromatography on silica gel (100-200 mesh silica gel, 0-10% DCM in MeOH) to give 3-((tert-butyldimethylsilyl)oxy)-1-methyl-6-(4,4,5,5-tetramethyl-1,3,2-dioxaborolan-2-yl)-1H-pyrazolo[4,3-b]pyridine (140 mg, 360 μmol, 72.4%) as a solid.

### Step E. 6-(3-(3-hydroxy-1-methyl-1H-pyrazolo[4,3-b]pyridin-6-yl)isoquinolin-8-yl)-4-isopropyl-1,3-dimethyl-1,3-dihydro-2H-benzo[d]imidazol-2-one

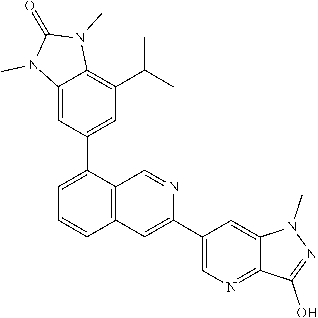 [SMILES: CC(C)c1cc(-c2cccc3cc(-c4cnc5c(O)nn(C)c5c4)ncc23)cc2c1n(C)c(=O)n2C]

To a solution of 3-((tert-butyldimethylsilyl)oxy)-1-methyl-6-(4,4,5,5-tetramethyl-1,3,2-dioxaborolan-2-yl)-1H-pyrazolo[4,3-b]pyridine (325 mg, 834 μmol), PdCl₂(dppf) (61.0 mg, 083.4 μmol), 8-(7-isopropyl-1,3-dimethyl-2-oxo-2,3-dihydro-1H-benzo[d]imidazol-5-yl)isoquinolin-3-yl trifluoromethanesulfonate (400 mg, 834 μmol) in 1,4-dioxane (5.00 mL) and water (250 μL) was added CsF (380 mg, 2.50 mmol), the mixture was stirred for 3 hours. The resulting solution was diluted with 40 mL of H₂O and extracted with EtOAc (40 mL×3). The combined organic layers were washed with brine, dried over Na₂SO₄ and concentrated under reduced pressure. The residue was purified by flash column chromatography on silica gel (100-200 mesh silica gel, 0-60% EA in PE) to give 6-(3-(3-hydroxy-1-methyl-1H-pyrazolo[4,3-b]pyridin-6-yl)isoquinolin-8-yl)-4-isopropyl-1,3-dimethyl-1,3-dihydro-2H-benzo[d]imidazol-2-one as a solid.

LC-MS (ESI): mass calcd. for C₂₈H₂₆N₆O₂: 478.2. m/z found: 479.1 [M+H]⁺.

### Step F. 3-(4-iodo-J-oxoisoindolin-2-yl)-1-((2-(trimethylsilyl)ethoxy)methyl)piperidine-2,6-dione

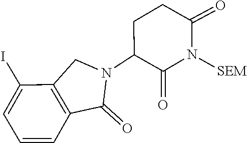 [SMILES: C[Si](C)(C)CCOCN1C(=O)CCC(N2Cc3c(I)cccc3C2=O)C1=O]

To a solution of 3-(4-iodo-1-oxoisoindolin-2-yl)piperidine-2,6-dione (1.40 g, 3.78 mmol) in DMF (20.0 mL) was added DBU (864 mg, 855 μL, 5.67 mmol) and SEM-Cl (946 mg, 1.00 mL, 5.67 mmol) at 25° C. The solution was stirred at 25° C. for 16 hours. The reaction was quenched with brine. The mixture was filtered and extracted with EA. The organic layer was dried over anhydrous Na₂SO₄, concentrated in vacuum to give crude product was purified by Flash (PE/EA 1:1) to give the 3-(4-iodo-1-oxoisoindolin-2-yl)-1-((2-(trimethylsilyl)ethoxy)methyl)piperidine-2,6-dione (1.30 g, 2.60 mmol, 68.7%) as a white solid.

¹H NMR (400 MHz, DMSO) δ 8.06 (d, J=7.6 Hz, 1H), 7.80 (d, J=7.4 Hz, 1H), 7.37 (t, J=7.7 Hz, 1H), 5.31 (dd, J=13.5, 5.1 Hz, 1H), 5.08 (dd, J=21.8, 9.8 Hz, 2H), 4.32 (d, J=17.3 Hz, 1H), 4.12 (d, J=17.3 Hz, 1H), 3.60-3.50 (m, 2H), 3.14-3.00 (m, 1H), 2.86-2.77 (m, 1H), 2.51-2.42 (m, 1H), 2.13-2.03 (m, 1H), 0.90-0.81 (m, 2H), 0.02-−0.03 (m, 9H).

### Step G: 3-(4-(3-hydroxyprop-1-yn-1-yl)-1-oxoisoindolin-2-yl)-1-((2-(trimethylsilyl)ethoxy)methyl) piperidine-2,6-dione

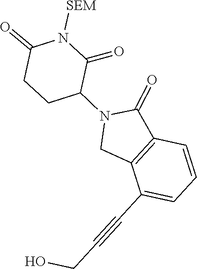 [SMILES: C[Si](C)(C)CCOCN1C(=O)CCC(N2Cc3c(C#CCO)cccc3C2=O)C1=O]

To a solution of 3-(4-bromo-1-oxoisoindolin-2-yl)-1-((2-(trimethylsilyl)ethoxy) methyl) piperidine-2,6-dione (1.00 g, 2.21 mmol) and bis-(triphenylphosphino)-palladium chloride (155 mg, 221 μmol) in DMF (5.00 mL) was added CuI (42.0 mg, 221 μmol) and TEA (2.23 g, 3.07 mL, 22.1 mmol) at 80° C. After stirred for 30 minutes, prop-2-yn-1-ol (371 mg, 6.62 mmol) was added into the reaction. The reaction was stirred at 80° C. for 16 hours under N₂. The reaction was quenched by water extracted with EtOAc. The organic phase was washed with brine, dried over Na₂SO₄, filtered and filtrate was concentrated in vacuum to give a residue. The residue was purified by TLC (SiO₂, PE:EA=1:1) to give the 3-(4-(3-hydroxyprop-1-yn-1-yl)-1-oxoisoindolin-2-yl)-1-((2-(trimethylsilyl)ethoxy)methyl)piperidine-2,6-dione (550 mg, 1.28 mmol, 58.2%) as a yellow oil.

LC-MS (ESI): mass calcd. for C₂₂H₂₈N₂O₅Si: 428.2. m/z found: 429.1 [M+H]⁺.

### Step H. 3-(4-(3-((6-(8-(7-isopropyl-1,3-dimethyl-2-oxo-2,3-dihydro-1H-benzo[d]imidazol-5-yl)isoquinolin-3-yl)-1-methyl-1H-pyrazolo[4,3-b]pyridin-3-yl)oxy)prop-1-yn-1-yl)-1-oxoisoindolin-2-yl)-1-((2-(trimethylsilyl)ethoxy)methyl)piperidine-2,6-dione

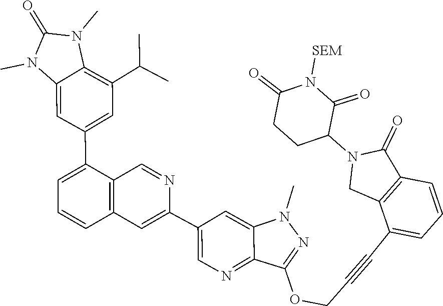 [SMILES: CC(C)c1cc(-c2cccc3cc(-c4cnc5c(OCC#Cc6cccc7c6CN(C6CCC(=O)N(COCC[Si](C)(C)C)C6=O)C7=O)nn(C)c5c4)ncc23)cc2c1n(C)c(=O)n2C]

To a stirred mixture of 6-(3-(3-hydroxy-1-methyl-1H-pyrazolo[4,3-b]pyridin-6-yl)isoquinolin-8-yl)-4-isopropyl-1,3-dimethyl-1,3-dihydro-2H-benzo[d]imidazol-2-one (20.0 mg, 41.8 μmol) in toluene (5.00 mL) was added 3-(4-(3-hydroxyprop-1-yn-1-yl)-1-oxoisoindolin-2-yl)-1-((2-(trimethylsilyl)ethoxy)methyl)piperidine-2,6-dione (17.9 mg, 41.8 μmol) and cyanomethylene tributylphosphorane (20.2 mg, 22.2 μL, 83.6 μmol) at room temperature. The mixture was stirred at 100° C. under N₂ for 4 hours. The reaction was quenched by water extracted with EtOAc. The organic phase was washed with brine, dried over Na₂SO₄, filtered and filtrate was concentrated in vacuum to give a residue. The residue was purified by TLC (SiO₂, PE:EA=1:1) to give 3-(4-(3-((6-(8-(7-isopropyl-1,3-dimethyl-2-oxo-2,3-dihydro-1H-benzo[d]imidazol-5-yl)isoquinolin-3-yl)-1-methyl-1H-pyrazolo[4,3-b]pyridin-3-yl)oxy)prop-1-yn-1-yl)-1-oxoisoindolin-2-yl)-1-((2-(trimethylsilyl)ethoxy)methyl)piperidine-2,6-dione as a colorless oil (25 mg, 67%).

LC-MS (ESI): mass calcd. for C₅₀H₅₂N₈O₆Si: 888.4. m/z found: 889.2 [M+H]⁺.

### Step I. 3-(4-(3-((6-(8-(7-isopropyl-1,3-dimethyl-2-oxo-2,3-dihydro-1H-benzo[d]imidazol-5-yl)isoquinolin-3-yl)-1-methyl-1H-pyrazolo[4,3-b]pyridin-3-yl)oxy)prop-1-yn-1-yl)-1-oxoisoindolin-2-yl)piperidine-2,6-dione

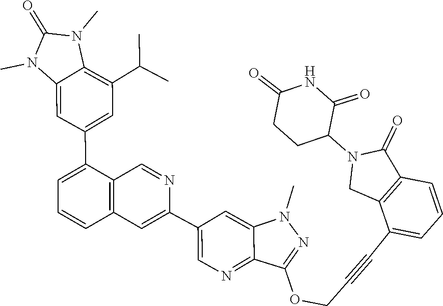 [SMILES: CC(C)c1cc(-c2cccc3cc(-c4cnc5c(OCC#Cc6cccc7c6CN(C6CCC(=O)NC6=O)C7=O)nn(C)c5c4)ncc23)cc2c1n(C)c(=O)n2C]

To a stirred solution of 3-(4-(3-((6-(8-(7-isopropyl-1,3-dimethyl-2-oxo-2,3-dihydro-1H-benzo[d]imidazol-5-yl)isoquinolin-3-yl)-1-methyl-1H-pyrazolo[4,3-b]pyridin-3-yl)oxy)prop-1-yn-1-yl)-1-oxoisoindolin-2-yl)-1-((2-(trimethylsilyl)ethoxy)methyl)piperidine-2,6-dione (40.0 mg, 45.0 μmol) in DCM (3.00 mL) was added TFA (4.44 g, 3.00 mL, 38.9 mmol) at room temperature. The mixture was stirred at room temperature under N₂ for 1 hour. The resulting solution was concentrated under reduced pressure. Then the residue was dissolved in MeCN (3.00 mL) was added TEA (4.55 mg, 6.27 μL, 45.0 μmol) at room temperature. The mixture was stirred at 70° C. under N₂ for 1 hour. The resulting solution was concentrated under reduced pressure. The residue was purified by prep-HPLC ((Triant C18 5 μm column, 0˜40% methane in H₂O with 0.1% HCOOH) to give 3-(4-(3-((6-(8-(7-isopropyl-1,3-dimethyl-2-oxo-2,3-dihydro-1H-benzo[d]imidazol-5-yl)isoquinolin-3-yl)-1-methyl-1H-pyrazolo[4,3-b]pyridin-3-yl)oxy)prop-1-yn-1-yl)-1-oxoisoindolin-2-yl)piperidine-2,6-dione (3.00 mg, 3.95 μmol, 8.79%) as a white solid.

LC-MS (ESI): mass calcd. for C₄₄H₃₈N₈O₅: 758.3. m/z found: 759.2 [M+H]⁺.

¹H NMR (400 MHz, DMSO) δ 11.01 (s, 1H), 9.41 (s, 1H), 9.32 (d, J=1.6 Hz, 1H), 8.80-8.75 (m, 2H), 8.08 (d, J=8.3 Hz, 1H), 7.98-7.87 (m, 1H), 7.79-7.73 (m, 2H), 7.67 (d, J=7.1 Hz, 1H), 7.57 (t, J=7.6 Hz, 1H), 7.30 (d, J=1.3 Hz, 1H), 7.24 (d, J=1.3 Hz, 1H), 5.46 (d, J=1.9 Hz, 2H), 5.12 (dd, J=13.3, 5.1 Hz, 1H), 4.36 (d, J=17.7 Hz, 1H), 4.22 (d, J=17.7 Hz, 1H), 4.02 (s, 3H), 3.79-3.71 (m, 1H), 3.66 (s, 3H), 3.39 (s, 3H), 2.93-2.82 (m, 1H), 2.55-2.52 (m, 1H), 2.34-2.26 (m, 1H), 2.01-1.96 (m, 1H), 1.35 (d, J=6.8 Hz, 6H).

### Example 3: 3-(4-(5-((6-(8-(7-isopropyl-1,3-dimethyl-2-oxo-2,3-dihydro-1H-benzo[d]imidazol-5-yl)isoquinolin-3-yl)-1-methyl-1H-pyrazolo[4,3-b]pyridin-3-yl)oxy)pent-1-yn-1-yl)-1-oxoisoindolin-2-yl)piperidine-2,6-dione

### Step A: 4-isopropyl-1,3-dimethyl-6-(3-(1-methyl-3-(pent-4-yn-1-yloxy)-1H-pyrazolo[4,3-b]pyridin-6-yl)isoquinolin-8-yl)-1,3-dihydro-2H-benzo[d]imidazol-2-one

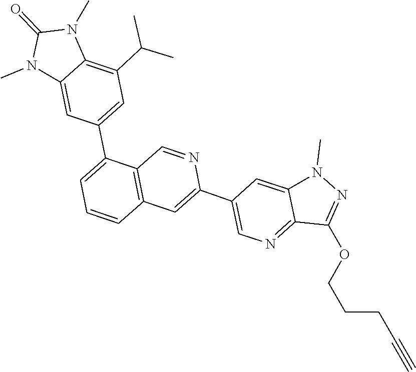 [SMILES: C#CCCCOc1nn(C)c2cc(-c3cc4cccc(-c5cc(C(C)C)c6c(c5)n(C)c(=O)n6C)c4cn3)cnc12]

To a stirred solution of 6-(3-(3-hydroxy-1-methyl-1H-pyrazolo[4,3-b]pyridin-6-yl)isoquinolin-8-yl)-4-isopropyl-1,3-dimethyl-1,3-dihydro-2H-benzo[d]imidazol-2-one (120 mg, 251 μmol) in DMF (8.00 mL) at 0° C. Then the 5-bromopent-1-yne (111 mg, 752 μmol) and K₂CO₃ (104 mg, 752 μmol) was added dropwise and the mixture was stirred at room temperature for an additional 3 hours. The resulting solution was diluted with 20 mL of water and extracted with EA (30 mL×3). The combined organic layers were washed with brine, dried over Na₂SO₄ and concentrated under reduced pressure. The residue was purified by flash column chromatography on silica gel (100-200 mesh silica gel, 0-60% EA in PE) to give 4-isopropyl-1,3-dimethyl-6-(3-(1-methyl-3-(pent-4-yn-1-yloxy)-1H-pyrazolo[4,3-b]pyridin-6-yl)isoquinolin-8-yl)-1,3-dihydro-2H-benzo[d]imidazol-2-one (100 mg, 0.17 mmol, 66%) as a brown solid.

LC-MS (ESI): mass calcd. for C₃₃H₃₂N₆O₂: 544.3. m/z found: 545.2 [M+H]⁺.

### Step B: 3-(4-(5-((6-(8-(7-isopropyl-1,3-dimethyl-2-oxo-2,3-dihydro-1H-benzo[d]imidazol-5-yl)isoquinolin-3-yl)-1-methyl-1H-pyrazolo[4,3-b]pyridin-3-yl)oxy)pent-1-yn-1-yl)-1-oxoisoindolin-2-yl)piperidine-2,6-dione

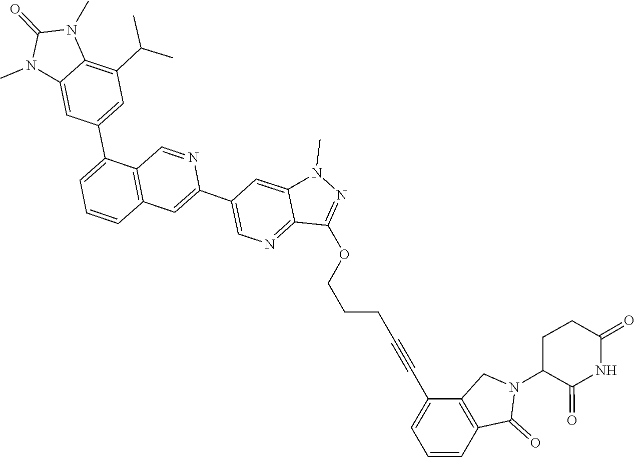 [SMILES: CC(C)c1cc(-c2cccc3cc(-c4cnc5c(OCCCC#Cc6cccc7c6CN(C6CCC(=O)NC6=O)C7=O)nn(C)c5c4)ncc23)cc2c1n(C)c(=O)n2C]

To a stirred solution of 3-(4-iodo-1-oxoisoindolin-2-yl)-1-((2-(trimethylsilyl)ethoxy) methyl)piperidine-2,6-dione (73.5 mg, 147 μmol), copper (I) iodide (2.80 mg, 14.7 μmol), triethylamine (149 mg, 1.47 mmol), bis-(triphenylphosphino)-palladium chloride (10.3 mg, 14.7 μmol) in DMF (5.00 mL) at 25° C. Then the 4-isopropyl-1,3-dimethyl-6-(3-(1-methyl-3-(pent-4-yn-1-yloxy)-1H-pyrazolo[4,3-b]pyridin-6-yl)isoquinolin-8-yl)-1,3-dihydro-2H-benzo[d]imidazol-2-one (80.0 mg, 1 Eq, 147 μmol) was added dropwise and the mixture was stirred at 25° C. for an additional 2 hours. The resulting solution was diluted with 20 mL of water and extracted with EA (30 mL×3). The combined organic layers were washed with brine, dried over Na₂SO₄ and concentrated under reduced pressure. The residue was purified by flash column chromatography on silica gel (100-200 mesh silica gel, 0-100% EA in PE) to give 3-(4-(5-((6-(8-(7-isopropyl-1,3-dimethyl-2-oxo-2,3-dihydro-1H-benzo[d]imidazol-5-yl)isoquinolin-3-yl)-1-methyl-1H-pyrazolo[4,3-b]pyridin-3-yl)oxy)pent-1-yn-1-yl)-1-oxoisoindolin-2-yl)-1-((2-(trimethylsilyl) ethoxy)methyl)piperidine-2,6-dione (75.0 mg, 74 μmol, 50%)

To a solution of 3-(4-(5-((6-(8-(7-isopropyl-1,3-dimethyl-2-oxo-2,3-dihydro-1H-benzo[d]imidazol-5-yl)isoquinolin-3-yl)-1-methyl-1H-pyrazolo[4,3-b]pyridin-3-yl)oxy)pent-1-yn-1-yl)-1-oxoisoindolin-2-yl)-1-((2-(trimethylsilyl)ethoxy)methyl)piperidine-2,6-dione (50.0 mg, 54.5 μmol) in DCM (5.00 mL) was added with TFA (5.34 mg, 1.00 mL, 154.5 μmol) and stirred at room temperature for 1 hour. The mixture was concentrated under reduced pressure to give 1-(hydroxymethyl)-3-(4-(5-((6-(8-(7-isopropyl-1,3-dimethyl-2-oxo-2,3-dihydro-1H-benzo[d]imidazol-5-yl)isoquinolin-3-yl)-1-methyl-1H-pyrazolo[4,3-b]pyridin-3-yl)oxy)pent-1-yn-1-yl)-1-oxoisoindolin-2-yl)piperidine-2,6-dione (40.0 mg, 44 μmol, 81%).

To a solution of 1-(hydroxymethyl)-3-(4-(5-((6-(8-(7-isopropyl-1,3-dimethyl-2-oxo-2,3-dihydro-1H-benzo[d]imidazol-5-yl)isoquinolin-3-yl)-1-methyl-1H-pyrazolo[4,3-b]pyridin-3-yl)oxy)pent-1-yn-1-yl)-1-oxoisoindolin-2-yl)piperidine-2,6-dione (50.0 mg, 61.2 μmol) in MeCN (3.00 mL), then ammonia water (0.09 g, 0.1 mL, 3 mmol) was added. The mixture was stirred at 25° C. for 2 hours under N₂ atmosphere. The residue was diluted with water, then adjusted to pH=3-4 with CF₃COOH. The resulting solution was concentrated under reduced pressure and purified by pre-HPLC (Instrument: SHIMADZU LH-40; Column: AZZOTA C18 COLUMN 30*250 mm 10 μm; Temperature: 25° C.; Inject number: 3; Wave length: 220 nm/254 nm; phase A: H₂O (0.1% FA); phase B: MeCN) to give 3-(4-(5-((6-(8-(7-isopropyl-1,3-dimethyl-2-oxo-2,3-dihydro-1H-benzo[d]imidazol-5-yl)isoquinolin-3-yl)-1-methyl-1H-pyrazolo[4,3-b]pyridin-3-yl)oxy)pent-1-yn-1-yl)-1-oxoisoindolin-2-yl)piperidine-2,6-dione (18.0 mg, 22 μmol, 36%).

LC-MS (ESI): mass calcd. for C₄₆H₄₂N₈O₅: 786.3. m/z found: 787.2 [M+H]⁺.

¹H NMR (400 MHz, DMSO) δ 10.99 (s, 1H), 9.41 (s, 1H), 9.26 (d, J=1.6 Hz, 1H), 8.74 (s, 2H), 8.08 (d, J=8.3 Hz, 1H), 7.90 (t, J=7.7 Hz, 1H), 7.73-7.63 (m, 3H), 7.52 (t, J=7.6 Hz, 1H), 7.32-7.21 (m, 2H), 5.12 (dd, J=13.3, 5.1 Hz, 1H), 4.57 (t, J=6.2 Hz, 2H), 4.47 (d, J=17.7 Hz, 1H), 4.33 (d, J=17.7 Hz, 1H), 3.97 (s, 3H), 3.76 (dt, J=13.3, 6.6 Hz, 1H), 3.66 (s, 3H), 3.39 (s, 3H), 2.94-2.83 (m, 1H), 2.73 (t, J=7.0 Hz, 2H), 2.44-2.31 (m, 1H), 2.17 (p, J=6.5 Hz, 2H), 2.06-1.96 (m, 1H), 1.35 (d, J=6.8 Hz, 6H).

### II. Biological Assessment

### In Vitro Assays:

The binding potency of the compounds to P300 and CBP were determined using HTRF assay technology. This assay uses full-length p300 expressed in baculovirus expression system with an N-terminal FLAG tag (Active Motif cat #81858). A 4× protein solution was prepared in an assay buffer consisting of 50 mM HEPES pH 7.5, 50 mM NaCl, 0.1% BSA, 1 mM TCEP, and 0.01% Brij-35. A 4× detection solution (MAb Anti FLAG M2-Tb cryptate, Cisbio, Cat #61FG2TLA) was also prepared in the same assay buffer. A fluorescent probe solution consisting of 5-(8-(7-acetyl-3-(tetrahydro-2H-pyran-4-yl)-5,6,7,8-tetrahydroimidazo[1,5-a]pyrazin-1-yl)isoquinolin-3-yl)-N-(2-(2-(2-(3-(3′,6′-dihydroxy-3-oxo-3H-spiro[isobenzofuran-1,9′-xanthen]-5-yl)thioureido)ethoxy) ethoxy)ethyl)picolinamide was made at 2× in the same assay buffer. The final concentrations in the assay were 2 nM of p300, 0.4 nM of Anti-FLAG Tb and 30 nM of the fluorescein probe.

Recombinant CREBBP (CBP), residues 1081-1197, was purchased from Active Motif (Cat #31373). This construct consists of an N-terminal His-tag and a C-terminal FLAG-tag, and is expressed in E. coli. Detection solution in this case consisted of Anti-6His Tb cryptate gold (Cisbio, Cat #61HI2TLB). The final concentrations in the assay were 0.2 nM of CBP (1081-1197), 0.4 nM of Anti-6His Tb and 15 nM of 5-(8-(7-acetyl-3-(tetrahydro-2H-pyran-4-yl)-5,6,7,8-tetrahydroimidazo[1,5-a]pyrazin-1-yl)isoquinolin-3-yl)-N-(2-(2-(2-(3-(3′,6′-dihydroxy-3-oxo-3H-spiro[isobenzofuran-1,9′-xanthen]-5-yl)thioureido)ethoxy)ethoxy)ethyl)picolinamide as a fluorescent probe.

To a 384-well assay plate (Perkin Elmer, Catalog #6008289) was added 5 μL of 4×p300 using a Dragonfly Discovery liquid dispenser (SPT Labtech). The plate was centrifuged at 211×g for 1 min. The plate was sealed and incubated for 15 min at room temperature. Next, 5 μL of 4× Anti-FLAG antibody was added into the assay plate using the Dragonfly Discovery liquid dispenser. Finally, 10 μL of 2× fluorescent probe was added to the assay plate using the same dispenser. The plate was centrifuged at 211×g for 1 min. The plate was resealed and incubated for 60 min at room temperature. Fluorescence values of the solutions were measured at an excitation wavelength of 495 nm and an emission wavelength of 520 nm using an Envision Multilabel Plate Reader (Perkin Elmer).

The CREBBP/CBP assay was performed in similar conditions to the p300 experiments described above.

p300 and CBP degradation potency and Dₘₐₓ were determined using in cell western (ICW) assay technology in H1299 cells or HiBit technology in A549 HiBit cells.

H1299 cell line was plated in Poly-L-Lysine coated and tissue culture treated 384-well flat, clear bottom microplate. Cells were plated at a cell density of 1.0×10⁵ cells per well in assay media comprised of RPMI 1640, 10% FBS, and 1% penicillin/streptomycin. Cells were placed in a 37° C., 5% CO₂, tissue culture incubator and left to equilibrate overnight. The next day cells were treated with serial dilutions of test compound made up of 10 mM stock solutions in DMSO. 384-well plates containing treated cells were then placed back into a 37° C., 5% CO₂, tissue culture incubator and incubated for 2 hrs for p300 and 48 hrs for CBP. After treatment, cell supernatant was removed from each plate and cells were fixed with PBS containing 30% Glyoxal for 1 hour at room temperature. Cell supernatant was then discarded, and cells were permeabilized with PBS containing 0.1% Triton X for 10 minutes at room temperature. Cell supernatant was removed and the cell permeabilization step was repeated two additional times with supernatant removed after each incubation. PBS containing 0.5% SDS was added to each well and incubated for 30 minutes at room temperature. Supernatant was removed and cells were blocked by adding LI-COR Blocking Buffer (LI-COR, catalog number: 927-60001) for 1 hour at room temperature. Cells supernatant was discarded, and LI-COR blocking buffer was added containing 1:1000 dilution of anti-p300 or anti-CBP primary antibody (Cell Signaling: anti-human p300 clone D8Z4E, catalog number 86377S; anti-human CBP clone D6C5, catalog number 7389S). Plates were covered with aluminum foil and incubated overnight at 4° C. Cell supernatant was discarded, and each plate washed 3 times with PBS containing 0.5% Tween with supernatant discarded after each wash. LI-COR blocking buffer was added containing IRDye 800CW goat anti-rabbit IgG secondary antibody (LI-COR; catalog number: 926-32211; 1:1000 dilution) and CellTag 700 stain (LI-COR; catalog number: 926-41090; 1:500 dilution) then incubated for 1 hour at room temperature. Cell supernatant was discarded, and each plate washed 3 times with PBS containing 0.5% Tween with supernatant discarded after each wash. Plates were left to air dry before being scanned with the LI-COR Odyssey CLx instrument.

A549 P300 HiBit and A549 CBP HiBit cells were plated in separate 384-well, TC treated, white-opaque, flat-bottomed microplate. Cells were plated at a cell density of 0.2×10⁴ cells per well in assay media comprised of F-12K, 10% heat inactivated FBS, and 1% penicillin/streptomycin. Assay microplates containing HiBit cells were placed in a 37° C., 5% CO₂, tissue culture incubator and left to equilibrate overnight. The next day cells were treated with serial dilutions of test compound made up of 10 mM stock solutions in DMSO. 384-Well plates containing treated cells were then placed back into a 37° C., 5% CO₂, tissue culture incubator. Assay microplates containing A549 P300 HiBit cells were incubated for 6 hrs post treatment before P300 destruction was assessed, while microplates containing A549 CBP HiBit cells were incubated for 6 hrs post treatment before CBP destruction was determined. On the day of each assay time point, microplates were removed from the tissue culture incubator and prepared HiBit Lytic detection buffer (Promega, cat #N3040) was added to each plate. Plates were covered with aluminum foil and incubated for ten minutes at room temperature on an orbital shaker. After incubation, luminescent signal of each plate was measured using the Envision instrument (Perkin Elmer).

TABLE A

| | P300 H1299 | P300 H1299 | CBP H1299 | CBP H1299 | | |
|---|---|---|---|---|---|---|
| Compound | 2 h ICW DC₅₀ | 2 h ICW Dₘₐₓ | 48 h ICW DC₅₀ | 48 h Dₘₐₓ | P300 BRD | CBP BRD |
| No. | (nM) | (%) | (nM) | (%) | IC₅₀ (nM) | IC₅₀ (nM) |
| 1 | D | E | B | G | E | G |
| 2 | G | G | E | G | — | — |

DC₅₀: “A” < 1.0 nM; 1.0 nM ≤ “B” < 2.0 nM; 2.0 nM ≤ “C” < 3.0 nM; 3.0 nM ≤ “D” < 4.0 nM; 4.0 nM ≤ “E” < 5.0 nM; 5.0 nM ≤ “F” < 6.0 nM; 6.0 nM ≤ “G” < 7.0 nM.
D_{Max}: “A” < 30%; 30% ≤ “B” < 40%; 40% ≤ “C” < 50%; 50% ≤ “D” < 60%; 60% ≤ “E” < 70%; 70% ≤ “F” < 80%; 80% ≤ “G” < 90%; 90% ≤ “H”.
IC₅₀: “A” < 50 nM; 50 nM ≤ “B” < 60 nM; 60 nM ≤ “C” < 70 nM; 70 nM ≤ “D” < 80 nM; 80 nM ≤ “E” < 90 nM; 90 nM ≤ “F” < 100 nM; 100 nM ≤ “G” < 110 nM.
“—” denotes that values were not determined or not available.

TABLE B

| Compound | P300 A549 2 h HiBit | P300 A549 2 h HiBit | CBP H1299 24 h HiBit | CBP A549 24 h |
|---|---|---|---|---|
| No. | AbsDC₅₀ (nM) | Dₘₐₓ (%) | AbsDC₅₀ (nM) | HiBit Dₘₐₓ (%) |
| 1 | D | E | B | F |
| 2 | D | F | G | B |

| Compound | P300 A549 6 h HiBit | P300 A549 6 h HiBit | CBP H1299 6 h HiBit | CBP A549 6 h |
|---|---|---|---|---|
| No. | AbsDC₅₀ (nM) | Dₘₐₓ (%) | AbsDC₅₀ (nM) | HiBit Dₘₐₓ (%) |
| 3 | A | G | A | G |

DC₅₀: “A” < 1.0 nM; 1.0 nM ≤ “B” < 2.0 nM; 2.0 nM ≤ “C” < 3.0 nM; 3.0 nM ≤ “D” < 4.0 nM; 4.0 nM ≤ “E” < 5.0 nM; 5.0 nM ≤ “F” < 6.0 nM; 6.0 nM ≤ “G” < 7.0 nM.
D_{Max}: “A” < 30%; 30% ≤ “B” < 40%; 40% ≤ “C” < 50%; 50% ≤ “D” < 60%; 60% ≤ “E” < 70%; 70% ≤ “F” < 80%; 80% ≤ “G” < 90%; 90% ≤ “H”.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

As used herein and in the appended claims, the singular forms “a,” “an,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth.

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A compound of Formula I: 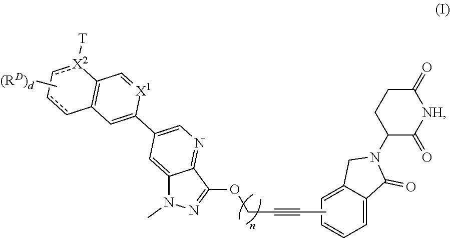
or a pharmaceutically acceptable salt, solvate, or stereoisomer thereof, wherein:
each [symbol] independently is a single bond or a double bond;
X¹ is N or CR^{X1};
R^{X1} is hydrogen or C₁-C₆ alkyl, wherein the C₁-C₆ alkyl is optionally substituted with one or more halogen, —CN, —OH, or —NH₂;
X² is N or C, as valency permits;
T is T-1 or T-2: 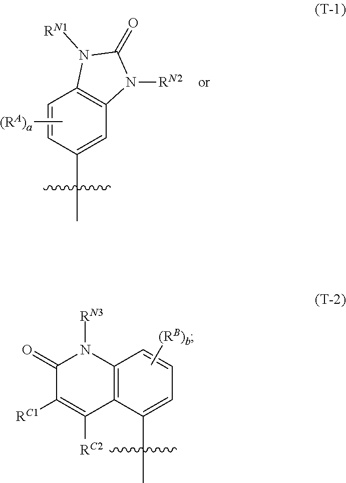
R^{N1} is hydrogen or C₁-C₆ alkyl, wherein the C₁-C₆ alkyl is optionally substituted with one or more halogen, —CN, —OH, or —NH₂;
R^{N2} is hydrogen or C₁-C₆ alkyl, wherein the C₁-C₆ alkyl is optionally substituted with one or more halogen, —CN, —OH, or —NH₂;
R^{N3} is hydrogen or C₁-C₆ alkyl, wherein the C₁-C₆ alkyl is optionally substituted with one or more halogen, —CN, —OH, or —NH₂;
each R^{A} independently is halogen, —CN, —OH, —NH₂, or C₁-C₆ alkyl, wherein the C₁-C₆ alkyl is optionally substituted with one or more halogen, —CN, —OH, or —NH₂;
a is an integer selected from 0 to 3;
each R^{B} independently is halogen, —CN, —OH, —NH₂, or C₁-C₆ alkyl, wherein the C₁-C₆ alkyl is optionally substituted with one or more halogen, —CN, —OH, or —NH₂;
b is an integer selected from 0 to 3;
R^{C1} is hydrogen or C₁-C₆ alkyl, wherein the C₁-C₆ alkyl is optionally substituted with one or more halogen, —CN, —OH, or —NH₂;
R^{C2} is hydrogen or C₁-C₆ alkyl, wherein the C₁-C₆ alkyl is optionally substituted with one or more halogen, —CN, —OH, or —NH₂;
each R^{D} independently is halogen, —CN, —OH, —NH₂, or C₁-C₆ alkyl, wherein the C₁-C₆ alkyl is optionally substituted with one or more halogen, —CN, —OH, or —NH₂;
d is an integer selected from 0 to 5, as valency permits; and
n is an integer selected from 1 to 5.

2. The compound of claim 1, wherein the compound is of Formula I-A: 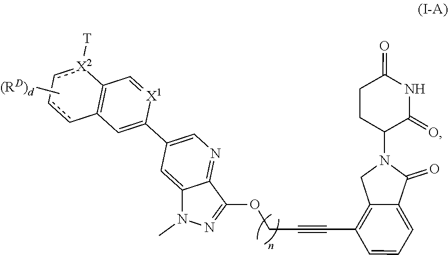
or a pharmaceutically acceptable salt, solvate, or stereoisomer thereof.

3. The compound of claim 1, wherein T is T-1.

4. The compound of claim 1, wherein a is 1.

5. The compound of claim 1, wherein T is T-1-a:

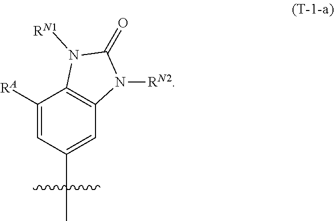

6. (canceled)

7. The compound of claim 1, wherein R^{N1} is C₁-C₆ alkyl optionally substituted with one or more halogen, —CN, —OH, or —NH₂.

8. The compound of claim 7, wherein R^{N1} is —CH₃.

9. (canceled)

10. The compound of claim 1, wherein R^{N2} is C₁-C₆ alkyl optionally substituted with one or more halogen, —CN, —OH, or —NH₂.

11. The compound of claim 10, wherein R^{N2} is —CH₃.

12. (canceled)

13. The compound of claim 1, wherein each R^{A} independently is C₁-C₆ alkyl optionally substituted with one or more halogen, —CN, —OH, or —NH₂.

14. The compound of claim 13, wherein each R^{A} independently is —CH(CH₃)₂.

15. The compound of claim 1, wherein T is

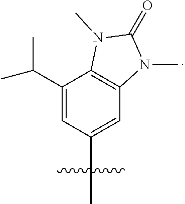 [SMILES: *c1cc(C(C)C)c2c(c1)n(C)c(=O)n2C]

16.-17. (canceled)

18. The compound of claim 1, wherein T is T-2-a:

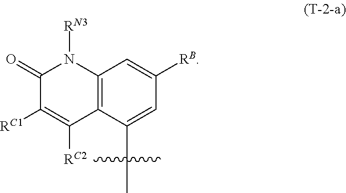

19.-30. (canceled)

31. The compound of claim 1, wherein X¹ is N.

32.-36. (canceled)

37. The compound of claim 1, wherein X² is C.

38.-39. (canceled)

40. The compound of claim 1, wherein d is 0.

41. The compound of claim 1, wherein n is an integer selected from 1 to 3.

42. The compound of claim 1 selected from the compounds in Table 1, or a pharmaceutically acceptable salt, solvate, or stereoisomer thereof.

43. A pharmaceutical composition comprising the compound of claim 1 and one or more pharmaceutically acceptable excipient.

44.-45. (canceled)

46. A method of (i) degrading a protein, or (ii) treating a p300-mediated disorder, comprising administering the compound of claim 1.

47. (canceled)
